(19) United States
(12) Patent Application Publication
(10) Pub. No.: US 2023/0018537 A1
(43) Pub. Date: Jan. 19, 2023

(54) PREDICTING AND ADDRESSING SEVERE DISEASE IN INDIVIDUALS WITH SEPSIS

(71) Applicant: The Henry M. Jackson Foundation for the Advancement of Military Medicine, Inc., Bethesda, MD (US)

(72) Inventors: Joost Brandsma, Bethesda, MD (US); Danielle Clark, Bethesda, MD (US); Rittal Mehta, Bethesda, MD (US); Deborah Striegel, Bethesda, MD (US)

(73) Assignee: The Henry M. Jackson Foundation for the Advancement of Military Medicine, Inc., Bethesda, MD (US)

(21) Appl. No.: 17/757,984

(22) PCT Filed: Dec. 24, 2020
(86) PCT No.: PCT/US2020/067038

§ 371 Date: Jun. 24, 2022

Related U.S. Application Data
(60) Provisional application No. 62/954,298, filed on Dec. 27, 2019.

(51) Int. Cl. G16H 50/30; G16B 20/00; G16B 40/20

(52) U.S. Cl. CPC G16H 50/30; G16B 20/00; G16B 40/20

(57) ABSTRACT

The present disclosure describes methods and systems for predicting severe disease in an individual with sepsis or at risk of developing sepsis, in order to prevent severe disease in an individual with sepsis. The present disclosure also describes methods of using topological data analysis and/or clustering to stratify sepsis based on biomolecular signatures and identify distinct phenotypes in sepsis patients.

### CROSS REFERENCE TO RELATED APPLICATIONS

This application claims the benefit of U.S. Provisional Patent Application 62/954,298, filed on Dec. 27, 2019, which is hereby incorporated by reference in its entirety.

### STATEMENT REGARDING FEDERALLY SPONSORED RESEARCH OR DEVELOPMENT

This invention was made with government support under grant N62645-14-2-0001. The government has certain rights in the invention.

### FIELD OF THE DISCLOSURE

Described herein are methods, systems, and computational environments for stratifying individuals with sepsis or at risk of developing sepsis, and for predicting severe disease in individuals with sepsis or at risk of developing sepsis. Also described are systems and methods for generating topological networks and clusters identifying disease-response phenotypes, systems and methods for selecting prognostic or diagnostic features and host biomarkers, and systems and methods for predicting clinical outcomes. Also described are methods of detecting panels of host biomarkers, methods of assessing risk factors in an individual with sepsis or at risk of developing sepsis, and methods of treating a patient determined to have an elevated risk of severe disease from sepsis.

### BACKGROUND OF THE DISCLOSURE

Expeditious and accurate information for clinical decision-making is critical for improving outcomes for infectious disease patients, particularly if a dysregulated host response to the infection leads to the potentially life-threatening organ dysfunction known as sepsis. Early recognition and characterization of an infection and the ensuing host response are essential components for preventing the development and/or mitigating the severity of sepsis. However, current diagnostic and prognostic assays are either insensitive or not expediently useful, if available at all. The use of specific host response biomarkers can improve our ability to quickly and accurately phenotype infectious disease states and predict their clinical course. This will be highly informative not just in traditional clinical settings, but also in low resource environments, military operations, and for at-home monitoring.

### SUMMARY OF THE DISCLOSURE

This Summary is provided to introduce a selection of concepts in a simplified form that are further described below in the Detailed Description. This Summary is not intended to identify all key features or essential features of the claimed subject matter, nor is it intended to be used alone as an aid in determining the scope of the claimed subject matter.

Described herein are methods of stratifying individuals with sepsis or at risk of developing sepsis; predicting severe disease in an individual with sepsis, including prior to the detection of symptoms thereof and/or prior to the onset of any detectable symptoms thereof; identifying disease-response phenotypes and associated diagnostic or prognostic host biomarker panels; and related methods of treatment targeted toward disease-response phenotypes.

The present disclosure also provides methods of treating individuals with sepsis determined to have an increased risk of severe disease, optionally before the onset of any detectable symptoms thereof, such as before there are perceivable, noticeable, or measurable signs of severe disease in the individual. Examples of treatment may include: initiation or broadening of antibiotic therapy, balancing fluids and electrolytes, renal replacement therapy, adjustment of mechanical ventilation, targeted or empiric anti-inflammatory or immunomodulatory drugs, hemodynamic adjustments, calcium channel blocker medications, or surgical intervention. Benefits of such early treatment may include: reduced severity or duration of symptoms, reduced need for organ support (e.g., ventilation, renal replacement therapy, or vasoactive medications), reduced length of stay in a hospital or intensive care unit, reduced risk of mortality, reduced long-term morbidity (e.g., time to returning to activities or quality of life), decreased incidence of long-term sequelae of infectious diseases (e.g., chronic kidney disease, cardiovascular disease, or chronic pulmonary disease), decreased re-hospitalization rates, and/or reduced medical costs.

In embodiments, there are provided methods for predicting severe disease in an individual with sepsis or at risk of developing sepsis, comprising: generating a discovery database storing first values of a plurality of clinical parameters and clinical outcomes associated with a plurality of first subjects; executing a plurality of data quality control algorithms to select a subset of clinical parameters from the plurality of clinical parameters; executing a plurality of topological data analysis and/or clustering algorithms for the plurality of subsets of clinical parameters; executing a plurality of feature selection machine learning and/or ensemble learning models based on a plurality of classification and/or time-to-event analysis algorithms; and outputting a model for predicting severe disease in the individual with sepsis or at risk of developing sepsis.

In embodiments, there are provided methods for generating a model predicting severe disease in an individual with sepsis or at risk of developing sepsis, comprising: generating a discovery database storing first values of a plurality of clinical parameters and clinical outcomes associated with a plurality of first subjects; executing a plurality of data quality control algorithms to select a subset of clinical parameters from the plurality of clinical parameters; executing a plurality of topological data analysis and/or clustering algorithms for the plurality of subsets of clinical parameters; executing a plurality of feature selection machine learning and/or ensemble learning models based on a plurality of classification and/or time-to-event analysis algorithms; and outputting a model for predicting severe disease in the individual with sepsis or at risk of developing sepsis.

In embodiments, there are provided methods for pre-processing data that is stored in the discovery database, including: determining that a first value of at least one of the plurality of clinical parameters is missing; estimating a reference value for the at least one of the plurality of clinical parameters that is missing; and storing the reference value as the first value of the at least one of the plurality of clinical parameters in the discovery database.

In embodiments, the plurality of data quality control algorithms comprise at least one of: differential expression algorithms, principal component analysis, k-nearest neighbor imputation algorithms, three-sigma rule algorithms, and empirical Bayes method algorithms. While these algorithms are enumerated for data quality control, many others are contemplated.

In embodiments, the clinical parameter data is stratified using topological data analysis and/or cluster analysis, wherein disease-response phenotypes are defined based on the identified clusters.

In embodiments, the cluster analysis comprises at least one of: k-means clustering, hierarchical clustering, nearest neighbor clustering, non-linear clustering (e.g., t-distributed stochastic neighbor embedding), consensus clustering, or spectral clustering. While these algorithms are enumerated for cluster analysis, many others are contemplated.

In embodiments, topological data analysis uses the Mapper algorithm as an alternative to canonical cluster analysis. A topological network is generated in which individuals or samples group together based on their similarities in the plurality of subsets of clinical parameters, as well as the algebraic topology of the same data. Clusters are then delineated based on the persistence homology of node density and connectivity (edges).

In embodiments, the feature selection machine learning models comprise at least one of: unsupervised machine learning algorithm, supervised machine learning algorithm, minimum redundancy maximum relevance, Student's t-test, Mann-Whitney U test, random forest, logistic regression, or neural networks.

The feature selection ensemble learning models include combinations of the models described herein for cluster analysis and machine learning. In embodiments, the feature selection ensemble learning models may comprise: cluster analysis, unsupervised machine learning algorithm, supervised machine learning algorithm, minimum redundancy maximum relevance, Student's t-test, Mann-Whitney U test, random forest, logistic regression, neural networks, or a combination thereof. Ensembles may also comprise: Bayes optimal classifier, classification and regression tree, bootstrap aggregating, boosting, Bayesian model averaging, Bayesian model combination, a bucket of models, stacking, or a combination thereof.

In embodiments, the plurality of biological parameters comprise one or more protein data markers, one or more nucleic acid data markers, one or more metabolite data markers, one or more clinical outcomes data, one or more administrative health data, or a combination thereof.

In embodiments, there are provided systems for generating a machine learning engine for predicting severe disease in an individual with sepsis or at risk of developing sepsis, comprising: one or more processors; a memory; a communication platform; a discovery database configured to store first values of a plurality of clinical parameters and clinical outcomes associated with a plurality of first subjects; a machine learning engine configured to: execute a plurality of data quality control algorithms to select a subset of clinical parameters from the plurality of clinical parameters; execute a plurality of topological data analysis and/or clustering algorithms for the plurality of subsets of clinical parameters; execute a plurality of feature selection machine learning and/or ensemble learning models based on a plurality of classification and/or time-to-event analysis algorithms; output a model for predicting severe disease in the individual with sepsis or at risk of developing sepsis.

In embodiments, there are provided systems for predicting severe disease in an individual with sepsis or at risk of developing sepsis, comprising: one or more processors; a memory; a communication platform; a discovery database configured to store first values of a plurality of clinical parameters and the clinical outcomes associated with a plurality of first subjects with sepsis or at risk of developing sepsis; a machine learning engine configured to pre-train a model for severe disease in an individual with sepsis or at risk of developing sepsis, wherein the model is pre-trained by performing operations comprising: executing a plurality of data quality control algorithms to select a subset of clinical parameters from the plurality of clinical parameters; executing a plurality of topological data analysis and/or clustering algorithms for the plurality of subsets of clinical parameters; executing a plurality of feature selection machine learning and/or ensemble learning models based on a plurality of classification and/or time-to-event analysis algorithms; outputting a model for predicting severe disease in the individual with sepsis or at risk of developing sepsis; and a prediction engine configured to receive, from a second individual, a second value of at least one clinical parameter of a plurality of clinical parameters; execute the pre-trained model for predicting severe disease of the second individual using the second value of at least one clinical parameter; and a display device configured to output the predicted outcome of the second individual.

In embodiments, there is provided a non-transitory computer-readable medium having information recorded thereon for generating a model for predicting severe disease in an individual with sepsis or at risk of developing sepsis, wherein the information, when read by a computer, causes the computer to perform operations of: generating a discovery database storing first values of a plurality of clinical parameters and clinical outcomes associated with a plurality of first subjects; executing a plurality of data quality control algorithms to select a subset of clinical parameters from the plurality of clinical parameters; executing a plurality of topological data analysis and/or clustering algorithms for the plurality of subsets of clinical parameters; executing a plurality of feature selection machine learning and/or ensemble learning models based on a plurality of classification and/or time-to-event analysis algorithms; outputting a model for predicting severe disease in the individual with sepsis or at risk of developing sepsis.

### BRIEF DESCRIPTION OF THE SEVERAL VIEWS OF THE DRAWINGS

The present disclosure can be better understood by reference to the following drawings. The drawings are merely exemplary to illustrate certain features that may be used singularly or in combination with other features, and the present disclosure should not be limited to the embodiments shown.

FIG. 1 depicts a method of predicting severe disease in individuals with sepsis or at risk of developing sepsis, through a process of acquisition of discovery data, data quality control in a data quality control engine, topological data analysis and/or clustering in a data stratification engine, feature selection and classification and/or time-to-event analyses in a feature selection and outcome modeling engine, and predicting severe disease in individuals with sepsis or at risk of developing sepsis in a prediction engine.

FIG. 2 illustrates a block diagram for a severe disease in sepsis prediction system for predicting severe disease in an individual with sepsis or at risk of developing sepsis, as described herein.

FIG. 3 illustrates a flow-chart for a severe disease in sepsis prediction system and the data flow at each stage of the system.

FIG. 4 illustrates an embodiment of a computational environment that involves a computing device, a network, and a remote device.

FIG. 5 illustrates an example of an Austere Environments Consortium for Enhanced Sepsis Outcomes (ACESO) flow chart for a sepsis host biomarker discovery phase.

FIG. 6 illustrates an example of topological data analysis networks of blood plasma gene expression in an ACESO discovery cohort.

FIG. 7 illustrates an example of topological data analysis networks of blood plasma protein expression in an ACESO discovery cohort.

FIG. 8 illustrates an example of a classification and regression tree output from an ensemble machine learning model prognosing risk of hospital admission in COVID-19 patients based on blood cytokine levels and basic demographics.

### DETAILED DESCRIPTION

The following detailed description is presented to enable any person skilled in the art to make and use the subject of the application. For purposes of explanation, specific nomenclature is set forth to provide a thorough understanding of the present disclosure. However, it will be apparent to one skilled in the art that these specific details are not required to practice the subject of the application. Descriptions of specific applications are provided only as representative examples. The present application is not intended to be limited to the embodiments shown, but is to be accorded the widest possible scope consistent with the principles and features disclosed herein.

The present disclosure provides methods of predicting severe disease and adjusting treatments for individuals with sepsis or at risk of developing sepsis, optionally before the onset of detectable symptoms thereof, such as before there are perceivable, noticeable, or measurable signs of severe disease in the individual. The individuals may be undergoing established treatment, and based on the clinical outcome predicted by the methods described herein adjustment can be made for more appropriate treatment. The present disclosure provides methods for predicting severe disease and adjusting treatments for individuals with sepsis or at risk of developing sepsis that are applicable to most, if not all, populations in different parts of the world.

The present disclosure also provides methods of treating individuals with sepsis determined to have an increased risk of severe disease, optionally before the onset of detectable symptoms thereof, such as before there are perceivable, noticeable or measurable signs of severe disease in the individual. Examples of treatment may include: initiation or broadening of antibiotic therapy, balancing fluids and electrolytes, renal replacement therapy, adjustment of mechanical ventilation, targeted or empiric anti-inflammatory or immunomodulatory drugs, hemodynamic adjustments, calcium channel blocker medications, or surgical intervention. Benefits of such early treatment may include: reduced severity or duration of sepsis, reduced need for organ support (e.g., ventilation, renal replacement therapy, or vasoactive medications), reduced length of stay in a hospital or intensive care unit, reduced risk of mortality, reduced long-term morbidity (e.g., time to returning to activities or quality of life), decreased incidence of long-term sequelae of infectious diseases (e.g., chronic kidney disease, cardiovascular disease, or chronic pulmonary disease), decreased re-hospitalization rates, and/or reduced medical costs. In embodiments, adjusting current treatment comprises changing dose of current antibiotic, changing to a different antibiotic, changing dose of non-steroidal anti-inflammatory drugs, or initiating or adjusting insulin therapy.

The present disclosure also provides using the methods described herein to monitor patients to help clinicians make decisions on adjusting treatments, when necessary.

Technical and scientific terms used herein have the meanings commonly understood by one of ordinary skill in the art to which the present disclosure pertains, unless otherwise defined.

As used herein, the singular forms “a,” “an,” and “the” designate both the singular and the plural, unless expressly stated to designate the singular only.

The terms “administer,” “administration,” or “administering” as used herein refer to (1) providing, giving, dosing and/or prescribing, such as by either a health professional or his or her authorized agent or under their direction, and (2) putting into, taking or consuming, such as by a health professional or the individual, and is not limited to any specific dosage forms or routes of administration, unless otherwise stated.

The terms “treat”, “treating” or “treatment”, as used herein, include alleviating, abating or ameliorating sepsis or one or more symptoms thereof, whether or not sepsis is considered to be “cured” or “healed” and whether or not all symptoms are fully resolved.

The terms “ameliorating” or “preventing” progression of sepsis include alleviating or preventing the development of one or more symptoms thereof, or impeding or preventing an underlying mechanism of severe disease, and achieving any therapeutic and/or prophylactic benefit.

As used herein, the term “sepsis” refers to the potentially life-threatening physical reaction of the host to an infection. The way sepsis is defined clinically continues to evolve, but recent definitions include the 2001 SCCM/ESICM/ACCP/ATS/SIS “Sepsis-2”, and the 2016 SCCM/ESICM “Sepsis-3”. Both definitions, and any future updates to the clinical definitions or international standards for defining sepsis, apply here.

As used herein, the term “at risk of developing sepsis” refers to an individual being infected by a pathogen, which may result in them developing sepsis. Examples of pathogens include, but are not limited to: viruses (e.g., influenza, ebolaviruses, SARS-CoV-2), bacteria (e.g., Escherichia coli, Mycobacterium tuberculosis, Salmonella sp., Leptospira sp., Rickettsia sp., Burkholderia pseudomallei), fungi (e.g., Aspergillus sp., Candida sp., Histoplasma sp., Pneumocystis jirovecii), or parasites (e.g., Plasmodium sp., Trypanosoma cruzi). Whilst infection by a pathogen is a prerequisite for developing sepsis, it is understood that not all infected individuals go on to develop sepsis.

As used herein, the term “severe disease” is defined as sepsis with any degree of end organ damage (e.g., kidney, respiratory, or liver failure). Sepsis patients who go on to develop severe disease will require significant medical intervention (e.g., admission to a hospital or intensive care unit, ventilation, renal replacement therapy) in order to avert permanent physical damage, long-term sequelae, and/or death.

As used herein the terms “marker” and “biomarkers” are used interchangeably to refer to a measurable substance from a biological sample. For example, these can comprise one or more protein data markers, one or more nucleic acid data markers, one or more metabolite data markers, or a combination thereof. The term “host biomarker” further indicates that the measurable substance is derived from the infected individual, rather than the infecting pathogen.

As used herein, the term “stratification” refers to the division of a group of individuals into subgroups, based on one or more shared characteristics, such as derived from the observable or measured biological parameters. For example, the division can be based on a characteristic already known relevant to the outcome, such as age, sex, or having a pre-existing condition, or it can be based on clusters identified in observable or measured biological parameters using any of a variety of data cluster analysis techniques.

As used herein, the term “clustering” refers to the grouping of individuals or samples based on one or more shared characteristics, such as derived from the observable or measured biological parameters. For example, these can comprise one or more host biomarkers, one or more clinical outcomes data, one or more administrative health data, or a combination thereof. Clustering is performed using dedicated mathematical algorithms, here primarily via topological data analysis or cluster analysis methods.

As used herein, the term “data quality control” refers to analytic approaches including visual and mathematical approaches to cleaning data, reformatting data, applying missing data algorithms, normalizing data, standardizing data, and/or reducing the dimensionality of data based on specific criteria.

As used herein, the term “topological data analysis” or “TDA” refers to the analysis of datasets using techniques from topology, a study of the properties of a geometric space which allows defining continuous deformation of subspaces. Extraction of information from datasets that are high-dimensional, incomplete, and noisy is generally challenging. In practice, TDA methods such as the “Mapper” algorithm, enable dimensionality reduction, visualization and clustering of complex data sets.

As used herein, the term “ensemble learning” refers to the use of multiple learning algorithms described herein to obtain better predictive performance than could be obtained from any of the constituent learning algorithms alone.

As used herein, the terms “individual”, “subject”, “patient”, or “test individual” indicates a mammal, in particular a human or non-human primate. The test individual may or may not be in need of an assessment of sepsis and/or severe disease. In embodiments, the test individual is assessed prior to the detection of symptoms of sepsis. In embodiments, the test individual is assessed prior to the onset of any detectable symptoms of sepsis. In embodiments, the test individual does not have detectable symptoms of any type of sickness or condition. In embodiments, the test individual has an exposure, injury, wound, or condition that puts them at risk of developing sepsis, such as: having a viral or bacterial infection, such as but not limited to: urinary tract infection, meningitis, endocarditis, or septic arthritis; undergoing a medical surgical or dental procedure; having an open wound or trauma, such as but not limited to: a blast injury, a crush injury, an extremity wound, a gunshot wound, or a wound received in combat; suffering a nosocomial infection; having undergone medical interventions such as central line placement or intubation; having diabetes; being HIV positive; undergoing hemodialysis; and/or undergoing an organ transplant procedure (donor or receiver). In embodiments, the individual does not have a condition that puts them at risk of severe disease from sepsis, prior to application of the methods described herein. In embodiments, the individual has a condition that puts them at risk of severe disease from sepsis.

As used herein, the term “clinical outcome” indicates a measurable status or change in the health, function or quality of life of an individual with sepsis or at risk of developing sepsis. Examples include, but are not limited to: severity or duration of symptoms, need for organ support (e.g., ventilation, renal replacement therapy, or vasoactive medications), response to treatment, admission to a hospital or intensive care unit, length of stay in a hospital or intensive care unit, mortality, long-term morbidity (e.g., time to returning to activities or quality of life), incidence of long-term sequelae of infectious diseases (e.g., chronic kidney disease, cardiovascular disease, or chronic pulmonary disease), and re-hospitalization. Clinical outcomes may be recorded as categorical data (e.g., “yes/no”, “presence/absence”, an ordinal scale), continuous data (e.g., blood pressure), temporal data (e.g., duration of symptoms, days hospitalized), or time-to-event data (e.g., days to death, time to return to normal daily activities).

As used herein, the term “increased risk” or “elevated risk” indicates that the test individual has an increased chance of severe disease from sepsis. In embodiments, the reference individual is the test individual at an earlier time point, including prior to having an exposure, injury, wound, or condition that puts them at risk of severe disease from sepsis, or at an earlier point in time after having such an exposure, injury, wound, or condition. The increased risk may be relative or absolute and may be expressed qualitatively or quantitatively. For example, an increased risk may be expressed as simply determining the individual's risk profile and placing them in an “increased risk” category, based upon previous studies. Alternatively, a numerical expression of the individual's increased risk may be determined based upon the risk profile. As used herein, examples of expressions of an increased risk include, but are not limited to: odds, probability, odds ratio, p-value, attributable risk, biomarker index score, relative frequency, positive predictive value, negative predictive value, risk, relative risk, hazard, and hazard ratio. Risk may be determined based on predicting a specific clinical outcome in the individual; for example, predicted outcome may include an indication of whether the individual will or will not experience a specific clinical event within a specific timeframe, or an indication of a likelihood that the individual will or will not experience a specific clinical event within a specific timeframe.

For example, the association between an individual's risk profile and the likelihood of severe disease from sepsis may be measured by an odds ratio (OR) and by the relative risk (RR). If P(R+) is the probability of experiencing a mortal event for individuals with the risk profile (R) and P(R−) is the probability of experiencing a specific clinical outcome for individuals without the risk profile, then the relative risk is the ratio of the two probabilities: RR=P(R+)/P(R−).

The attributable risk (AR) can also be used to express an increased risk. The AR describes the proportion of individuals in a population exhibiting a specific outcome (e.g., mortality, hospitalization, or long-term sequelae) to a specific member of the risk profile. AR may also be important in quantifying the role of individual components (specific member) in condition etiology and in terms of the public health impact of the individual risk factor. The public health relevance of the AR measurement lies in estimating the proportion of cases of a clinical outcome among individuals in the population that could be prevented if the profile or individual factor were absent. AR may be determined as follows: AR=PE(RR−1)/(PE(RR−1)+1), where AR is the risk attributable to a profile or individual factor of the profile, and PE is the frequency of exposure to a profile or individual component of the profile within the population at large. RR is the relative risk, which can be approximated with the odds ratio when the profile or individual factor of the profile under study has a relatively low incidence in the general population.

Clinical parameters include various factors associated with an individual experiencing symptoms of a disease or condition, or in measurable changes in health, function, or quality of life. Examples of clinical parameters of an individual include, but are not limited to: proteins, nucleic acids, metabolites, clinical outcomes, clinical laboratory data, physiological monitoring data, and administrative health data.

Examples of the nucleic acids include, but are not limited to the level of any one or more of the following in a biological sample from the individual: adhesion G protein-coupled receptor E1 (ADGRE1), adrenoceptor β2 (ADRB2), angiotensin II receptor associated protein (AGTRAP), AKT serine/threonine kinase 1 (AKT1), 5′-aminolevulinate synthase 2 (ALAS2), alkaline phosphatase, biomineralization associated (ALPL), ankyrin repeat domain 22 (ANKRD22), annexin A3 (ANXA3), arginase 1 (ARG1), BCL2 like 1 (BCL2L1), BMX non-receptor tyrosine kinase (BMX), chromosome 6 open reading frame 62 (C6orf62), carbonic anhydrase 2 (CA2), C-C motif chemokine ligand 5 (CCL5), C-C motif chemokine receptor 3 (CCR3), CD4 molecule (CD4), CD24 molecule (CD24), CD177 molecule (CD177), CD274 molecule (CD274), cell division cycle 34, ubiqiutin conjugating enzyme (CDC34), complement factor D (CFD), chitinase 3 like 1 (CHI3L1), carbohydrate sulfotransferase 2 (CHST2), C-type lectin domain family 4 member E (CLEC4E), cytidine/uridine monophosphate kinase 2 (CMPK2), cytochrome C oxidase assembly factor 1 homolog (COA1), carnitine palmitoyltransferase 1A (CPT1A), carboxypeptidase vitellogenic like (CPVL), chondroitin sulfate N-acetylgalactosaminyltransferase 1 (CSGALNACT1), cystatin C (CST3), C-X3-C motif chemokine receptor 1 (CX3CR1), DNA damage inducible transcript 4 (DDIT4), defensin α3 (DEFA3), defensin α4 (DEFA4), DNA J heat shock protein family (Hsp40) member C1 (DNAJC1), DNA damage regulated autophagy modulator 1 (DRAM1), deoxyuridine triphosphatase (DUT), dual specificity tyrosine phosphorylation regulated kinase 3 (DYRK3), erythrocyte membrane protein band 4.2 (EPB42), family with sequence similarity 174 member C (FAM174C), F-box and WD repeat domain containing 2 (FBXW2), Fc receptor like 5 (FCRL5), ferrochelatase (FECH), fibroblast growth factor binding protein 2 (FGFBP2), Fms related receptor tyrosine kinase 3 (FLT3), formyl peptide receptor 1 (FPR1), GATA binding protein 1 (GATA1), GTPase, IMAP family member 4 (GIMAP4), GTPase, IMAP family member 7 (GIMAP7), GTPase, IMAP family member 8 (GIMAP8), G protein subunit γ2 (GNG2), granulysin (GNLY), G protein-coupled receptor 65 (GPR65), growth factor receptor bound protein 10 (GRB10), glutathione S-transferase κ1 (GSTK1), H3 histone pseudogene 6 (H3F3AP4), hemoglobin subunit α2 (HBA2), hemogen (HEMGN), HECT and RLD domain containing E3 ubiquitin protein ligase family member 6 (HERC6), H3.2 histone [putative](HIST2H3PS2), major histocompatibility complex, class I, B (HLA-B), major histocompatibility complex, class II, DQ β1 (HLA-DQB1), high mobility group box 2 (HMGB2), 15-hydroxyprostaglandin dehydrogenase (HPGD), hydrogen voltage gated channel 1 (HVCN1), isoamyl acetate hydrolyzing esterase 1 [putative] (IAH1), intercellular adhesion molecule 1 (ICAM1), immediate early response 5 (IER5), interferon α inducible protein 6 (IFI6), interferon α inducible protein 27 (IFI27), interferon induced protein 44 (IFI44), interferon induced protein with tetratricopeptide repeats 1 (IFIT1), interferon induced protein with tetratricopeptide repeats 2 (IFIT2), interleukin 1βR (IL1B), interleukin 1 receptor type 1 (IL1RA), interleukin 1 receptor type 2 (IL1 R2), interleukin 10 receptor subunit α (IL10RA), interaction protein for cytohesin exchange factors 1 (IPCEF1), interferon regulatory factor 2 binding protein 2 (IRF2BP2), ISG15 ubiquitin like modifier (ISG15), JUN proto-oncogene, AP-1 transcription factor subunit (JUN), potassium voltage-gated channel subfamily E regulatory subunit 1 (KCNE1), kinesin light chain 3 (KLC3), kelch like family member 24 (KLHL24), kringle containing transmembrane protein 1 (KREMEN1), long intergenic non-protein coding RNA 861 (LINC00861), lymphocyte antigen 6 family member E (LY6E), MAPK associated protein 1 (MAPKAP1), mediator complex subunit 28 (MED28), MicroRNA 6724-4 (MIR6724-4), matrix metallopeptidase 8 (MMP8), multimerin 1 (MMRN1), myeloperoxidase (MPO), mannose receptor C type 2 (MRC2), mitochondrially encoded 12S rRNA (MT-RNR1), MX dynamin like GTPase 2 (MX2), nuclear factor, erythroid 2 like 3 (NFE2L3), 2′-5′-oligoadenylate synthetase 3 (OAS3), oleoyl-ACP hydrolase (OLAH), olfactomedin 4 (OLFM4), peptidase inhibitor 3 (PI3), phosphatidylinositol-4,5-bisphosphate 3-kinase catalytic subunit β (PIK3CB), PITH domain containing 1 (PITHD1), pyruvate kinase M1/2 (PKM), perilipin 2 (PLIN2), DNA polymerase δ interacting protein 3 (POLDIP3), RAL GTPase activating protein catalytic subunit α2 (RALGAPA2), RAN binding protein 9 (RANBP9), REST corepressor 1 (RCOR1), Rh associated glycoprotein (RHAG), RNA, U1 small nuclear 2 (RNU1-2), RNA, U1 small nuclear 4 (RNU1-4), ribosomal protein L37a (RPL37A), ribosomal protein L38 (RPL38), ribosomal protein S11 (RPS11), ribosomal protein S18 (RPS18), radical S-adenosyl methionine domain containing 2 (RSAD2), S100 calcium binding protein A8 (S100A8), S100 calcium binding protein A9 (S100A9), S100 calcium binding protein A12 (S100A12), SAM domain, SH3 domain and nuclear localization signals 1 (SAMSN1), Sin3A associated protein 30 (SAP30), strawberry notch homolog 1 (SBNO1), selenium binding protein 1 (SELENBP1), sialic acid binding Ig like lectin 10 (SIGLEC10), solute carrier family 25 member 6 (SLC25A6), solute carrier family 25 member 39 (SLC25A39), solute carrier family 39 member 8 (SLC39A8), solute carrier family 4 member 1 [Diego Blood Group] (SLC4A1), synuclein α (SNCA), small nucleolar RNA, H/ACA box 44 (SNORA44), superoxide dismutase 2 (SOD2), spectrin α, erythrocytic 1 (SPTA1), STE20 related adaptor β (STRADB), syntaxin 6 (STX6), switching B cell complex subunit SWAP70 (SWAP70), spectrin repeat containing nuclear envelope protein 2 (SYNE2), T-box transcription factor 21 (TBX21), TRAF interacting protein with forkhead associated domain (TIFA), toll like receptor 7 (TLR7), transmembrane and coiled-coil domain family 2 (TMCC2), transmembrane protein 35B (TMEM35B), transmembrane protein 273 (TMEM273), thymosin β10 (TMSB10), TNF α induced protein 6 (TNFAIP6), tyrosylprotein sulfotransferase 1 (TPST1), tripartite motif containing 4 (TRIM4), tetraspanin 5 (TSPAN5), tetratricopeptide repeat domain 9C (TTC9C), ubiquitin protein ligase E3 component N-recognin 5 (UBR5), UNC-93 homolog B1, TLR signaling regulator (UNC93B1), WASH complex subunit 2C (WASHC2C), XIAP associated factor 1 (XAF1), tyrosine 3-monooxygenase/tryptophan 5-monooxygenase activation protein E (YWHAH), and zinc finger With KRAB and SCAN domains 1 (ZKSCAN1).

In embodiments, the genes are protein-coding genes. In embodiments, the genes are at least one or more of: adrenoceptor β2 (ADRB2), CD177 molecule (CD177), carboxypeptidase vitellogenic like (CPVL), C-X3-C motif chemokine receptor 1 (CX3CR1), defensin α3 (DEFA3), Fc receptor like 5 (FCRL5), G protein subunit γ2 (GNG2), interleukin 10 receptor subunit α (IL10RA), kinesin light chain 3 (KLC3), oleoyl-ACP hydrolase (OLAH), pyruvate kinase M1/2 (PKM), radical S-adenosyl methionine domain containing 2 (RSAD2), STE20 related adaptor β (STRADB), tyrosylprotein sulfotransferase 1 (TPST1), tetraspanin 5 (TSPAN5), tetratricopeptide repeat domain 9C (TTC9C), or zinc finger with KRAB and SCAN domains 1 (ZKSCAN1).

Examples of the proteins include, but are not limited to the level of any one or more of the following in a biological sample from the individual: a disintegrin and metalloproteinase with thrombospondin motifs 13 (ADAMTS13), Angiopoietin-1 (ANGPT1), Angiopoietin-2 (ANGPT2), C-C chemokine receptor ligand 2/monocyte chemoattractant protein 1 (CCL2/MCP-1), C-C chemokine receptor ligand 3/macrophage inflammatory protein 1-alpha (CCL3/MIP-1-α), C-C chemokine receptor ligand 5/regulated on activation, normal T cell expressed and secreted (CCL5/RANTES), cluster of differentiation 163 (CD163), cluster of differentiation 40 ligand (CD40L), chitinase-3-like protein 1 (CHI3L1), C-reactive protein (CRP), C-X-C motif chemokine ligand 10/interferon gamma-induced protein 10 (CXCL10/IP-10), decoy receptor 3 (Dcr3), D-dimer, E-selectin (SELE), endoglin (ENG), fas receptor (FAS), ferritins, fibrinogens, granulocyte colony-stimulating factor (G-CSF), granulocyte-macrophage colony-stimulating factor (GM-CSF), (soluble) intercellular adhesion molecule 1 (ICAM-1), interferon gamma (IFNγ), interleukin 1 beta (IL-1β), interleukin-1 receptor antagonist (IL-1RA), (soluble) interleukin-2 receptor α (IL-2Rα), interleukin-4 (IL-4), interleukin-5 (IL-5), interleukin-6 (IL-6), interleukin-6 receptor α (IL-6Rα), interleukin-7 (IL-7), interleukin-8 (IL-8), interleukin-10 (IL-10), interleukin-12 ‘p70’ (IL-12 p70), interleukin-15 (IL-15), interleukin-16 (IL-16), interleukin-17A (IL-17A), interleukin-18 (IL-18), interleukin-18-binding protein (IL-18BP), interleukin-22 (IL-22), interleukin-27 (IL-27), lipocalin-2 (LCN-2), matrix metalloproteinase-8 (MMP-8), matrix metalloproteinase-9 (MMP-9), matrix metalloproteinase-10 (MMP-10), (soluble) macrophage mannose receptors, procalcitonin (PCT), (soluble) programmed death-ligand 1 (PD-L1), pentraxin 3 (PTX3), (soluble) receptor for advanced glycation end products (RAGE), resistin (RETN), serum amyloid A proteins (SAA), tyrosine kinase with immunoglobulin-like and EGF-like domains 1 (TIE1), tyrosine kinase with immunoglobulin-like and EGF-like domains 2 (TIE2), tissue inhibitor of metalloproteinases 1 (TIMP1), tissue inhibitor of metalloproteinases 2 (TIMP2), tissue inhibitor of metalloproteinases 3 (TIMP3), tissue inhibitor of metalloproteinases 4 (TIMP4), tumor necrosis factor receptor 1 (TNF-R1), tumor necrosis factor alpha (TNFα), tissue plasminogen activator (tPA), tissue plasminogen activator inhibitor 1 (tPAI-1), TNF-related apoptosis-inducing ligand (TRAIL), (soluble) triggering receptor expressed on myeloid cells 1 (TREM1), urokinase receptor (uPar), (soluble) vascular cell adhesion molecule 1 (VCAM-1), vascular endothelial growth factors (VEGF), (soluble) vascular endothelial growth factor receptor 1 (VEGFR-1), (soluble) vascular endothelial growth factor receptor 2 (VEGFR-2), and von Willebrand factor A2 domain (vWF-A2).

In embodiments, the proteins are at least one or more of: C-reactive protein (CRP), C-X-C motif chemokine ligand 10/interferon gamma-induced protein 10 (CXCL10/IP-10), D-dimer, ferritins, fibrinogens, (soluble) intercellular adhesion molecule 1 (ICAM-1), interferon gamma (IFNγ), interleukin-1 receptor antagonist (IL-1RA), interleukin-5 (IL-5), interleukin-6 (IL-6), interleukin-6 receptor α (IL-6Rα), interleukin-18 (IL-18), interleukin-18-binding protein (IL-18BP), lipocalin-2 (LCN-2), matrix metalloproteinase-8 (MMP-8), procalcitonin (PCT), (soluble) receptor for advanced glycation end products (RAGE), tumor necrosis factor receptor 1 (TNF-R1), tumor necrosis factor alpha (TNFα), vascular endothelial growth factors (VEGF), or von Willebrand factor A2 domain (vWF-A2).

Examples of the metabolites include, but are not limited to the level of any one or more of the following in a biological sample from the individual: fatty acyls and their constituent molecular species, glycerolipids and their constituent molecular species, glycerophospholipids and their constituent molecular species, sphingolipids and their constituent molecular species, sterol lipids and their constituent molecular species, prenol lipids and their constituent molecular species, saccharolipids and their constituent molecular species, polyketides and their constituent molecular species, carbohydrates and their constituent molecular species, organic acids and their derivatives and constituent molecular species, organo-heterocyclic compounds and their constituent molecular species, organo-oxygen compounds and their constituent molecular species, organo-nitrogen compounds and their constituent molecular species, amino acids and their constituent molecular species, peptides and their constituent molecular species, and nucleosides and their constituent molecular species.

In embodiments, the metabolites are at least one or more of: carnitine, acetylcarnitine, propionylcarnitine, malonylcarnitine, methylmalonylcarnitine, hydroxypropionylcarnitine, propenoylcarnitine, butyrylcarnitine, hydroxybutyrylcarnitine, fumarylcarnitine, valerylcarnitine, glutarylcarnitine, hydroxyvalerylcarnitine, tiglylcarnitine, hexanoylcarnitine, hydroxyhexanoylcarnitine, pimeloylcarnitine, decanoylcarnitine, decadienylcarnitine, tetradecenoylcarnitine, hydroxytetradecenoylcarnitine, hydroxytetradecadienylcarnitine, hexadecanoylcarnitine, hexadecenoylcarnitine, octadecanoylcarnitine, octadecenoylcarnitine, lysophosphatidylcholine with acyl residue sum C16:0, lysophosphatidylcholine with acyl residue sum C16:1, lysophosphatidylcholine with acyl residue sum C17:0, lysophosphatidylcholine with acyl residue sum C18:0, lysophosphatidylcholine with acyl residue sum C18:1, lysophosphatidylcholine with acyl residue sum C18:2, lysophosphatidylcholine with acyl residue sum C20:3, lysophosphatidylcholine with acyl residue sum C20:4, lysophosphatidylcholine with acyl residue sum C24:0, lysophosphatidylcholine with acyl residue sum C26:0, lysophosphatidylcholine with acyl residue sum C26:1, lysophosphatidylcholine with acyl residue sum C28:0, lysophosphatidylcholine with acyl residue sum C28:1, phosphatidylcholine with diacyl residue sum C24:0, phosphatidylcholine with diacyl residue sum C28:1, phosphatidylcholine with diacyl residue sum C30:0, phosphatidylcholine with diacyl residue sum C32:0, phosphatidylcholine with diacyl residue sum C32:1, phosphatidylcholine with diacyl residue sum C32:3, phosphatidylcholine with diacyl residue sum C34:1, phosphatidylcholine with diacyl residue sum C34:2, phosphatidylcholine with diacyl residue sum C34:3, phosphatidylcholine with diacyl residue sum C34:4, phosphatidylcholine with diacyl residue sum C36:0, phosphatidylcholine with diacyl residue sum C36:1, phosphatidylcholine with diacyl residue sum C36:2, phosphatidylcholine with diacyl residue sum C36:3, phosphatidylcholine with diacyl residue sum C36:4, phosphatidylcholine with diacyl residue sum C36:5, phosphatidylcholine with diacyl residue sum C36:6, phosphatidylcholine with diacyl residue sum C38:0, phosphatidylcholine with diacyl residue sum C38:3, phosphatidylcholine with diacyl residue sum C38:4, phosphatidylcholine with diacyl residue sum C38:5, phosphatidylcholine with diacyl residue sum C38:6, phosphatidylcholine with diacyl residue sum C40:2, phosphatidylcholine with diacyl residue sum C40:3, phosphatidylcholine with diacyl residue sum C40:4, phosphatidylcholine with diacyl residue sum C40:5, phosphatidylcholine with diacyl residue sum C40:6, phosphatidylcholine with diacyl residue sum C42:0, phosphatidylcholine with diacyl residue sum C42:1, phosphatidylcholine with diacyl residue sum C42:2, phosphatidylcholine with diacyl residue sum C42:4, phosphatidylcholine with diacyl residue sum C42:5, phosphatidylcholine with diacyl residue sum C42:6, phosphatidylcholine with acyl-alkyl residue sum C30:0, phosphatidylcholine with acyl-alkyl residue sum C30:1, phosphatidylcholine with acyl-alkyl residue sum C30:2, phosphatidylcholine with acyl-alkyl residue sum C32:1, phosphatidylcholine with acyl-alkyl residue sum C32:2, phosphatidylcholine with acyl-alkyl residue sum C34:0, phosphatidylcholine with acyl-alkyl residue sum C34:1, phosphatidylcholine with acyl-alkyl residue sum C34:2, phosphatidylcholine with acyl-alkyl residue sum C34:3, phosphatidylcholine with acyl-alkyl residue sum C36:0, phosphatidylcholine with acyl-alkyl residue sum C36:1, phosphatidylcholine with acyl-alkyl residue sum C36:2, phosphatidylcholine with acyl-alkyl residue sum C36:3, phosphatidylcholine with acyl-alkyl residue sum C36:4, phosphatidylcholine with acyl-alkyl residue sum C36:5, phosphatidylcholine with acyl-alkyl residue sum C38:0, phosphatidylcholine with acyl-alkyl residue sum C38:1, phosphatidylcholine with acyl-alkyl residue sum C38:2, phosphatidylcholine with acyl-alkyl residue sum C38:3, phosphatidylcholine with acyl-alkyl residue sum C38:4, phosphatidylcholine with acyl-alkyl residue sum C38:5, phosphatidylcholine with acyl-alkyl residue sum C38:6, phosphatidylcholine with acyl-alkyl residue sum C40:1, phosphatidylcholine with acyl-alkyl residue sum C40:2, phosphatidylcholine with acyl-alkyl residue sum C40:3, phosphatidylcholine with acyl-alkyl residue sum C40:4, phosphatidylcholine with acyl-alkyl residue sum C40:5, phosphatidylcholine with acyl-alkyl residue sum C40:6, phosphatidylcholine with acyl-alkyl residue sum C42:2, phosphatidylcholine with acyl-alkyl residue sum C42:3, phosphatidylcholine with acyl-alkyl residue sum C42:5, phosphatidylcholine with acyl-alkyl residue sum C44:3, phosphatidylcholine with acyl-alkyl residue sum C44:4, phosphatidylcholine with acyl-alkyl residue sum C44:5, phosphatidylcholine with acyl-alkyl residue sum C44:6, hydroxysphingomyelin with acyl residue sum C14:1, hydroxysphingomyelin with acyl residue sum C16:1, hydroxysphingomyelin with acyl residue sum C22:1, hydroxysphingomyelin with acyl residue sum C22:2, hydroxysphingomyelin with acyl residue sum C24:1, sphingomyelin with acyl residue sum C16:0, sphingomyelin with acyl residue sum C16:1, sphingomyelin with acyl residue sum C18:0, sphingomyelin with acyl residue sum C18:1, sphingomyelin with acyl residue sum C20:2, sphingomyelin with acyl residue sum C24:0, sphingomyelin with acyl residue sum C24:1, sphingomyelin with acyl residue sum C26:0, sphingomyelin with acyl residue sum C26:1, hexoses [including glucose], alanine, arginine, asparagine, aspartate, citrulline, glutamine, glutamate, glycine, histidine, isoleucine, lysine, methionine, ornithine, phenylalanine, proline, serine, threonine, tryptophan, tyrosine, valine, asymmetric dimethylarginine, alpha-aminoadipic acid, creatinine, kynurenine, methionine sulfoxide, putrescine, sarcosine, symmetric dimethylarginine, spermidine, spermine, trans-4-hydroxyproline, or taurine.

Examples of clinical outcome data include, but are not limited to any one or more of the following: severity or duration of symptoms, time to symptom onset or abatement, need for organ support, duration of organ support, response to treatment, admission to a hospital or intensive care unit, length of stay in a hospital or intensive care unit, mortality, time to death, duration of morbidity (e.g., time to returning to normal daily activities or quality of life), incidence of long-term sequelae of infectious diseases, and re-hospitalization.

Examples of administrative health data include, but are not limited to any one or more of: baseline demographics (e.g., age, sex, ethnicity), physiological parameters (e.g., body mass index, heart rate, respiratory rate, body temperature), comorbid conditions including but not limited to immunocompromising conditions (e.g., history of chronic kidney disease, history of hepatic disease, pulmonary hypertension, dementia, having diabetes, being HIV positive, tobacco use, alcohol use, drug use, or pregnancy), past surgical history (e.g., central line placement, organ transplant donor or recipient), and environmental or social exposures (e.g., living situation, travel history, contact with livestock.

The clinical parameters may include one or more biological effectors and/or one or more non-biological effectors. As used herein, the term “biological effector” is used to mean a molecule, such as, but not limited to: a protein, a peptide, a carbohydrate, a complex lipid, a fatty acid, an amino acid, a biogenic amine, a nucleic acid, a glycoprotein, or a proteoglycan, that can be assayed. Specific examples of biological effectors can include: cytokines, growth factors, antibodies, hormones, cell surface receptors, cell surface proteins, lipid mediators, or carbohydrates. More specific examples of biological effectors include, but are not limited to the the genes, proteins, and metabolites described herein.

In embodiments, the biological effectors are soluble. In embodiments, the biological effectors are membrane-bound, such as a cell surface receptor. In embodiments, the biological effectors are intracellular. In embodiments the biological effectors are nucleic acids (e.g., messenger RNA, transfer RNA, micro RNA, long-noncoding RNA, silencing RNA, short hairpin RNA, or DNA). In embodiments, the biological effectors are detectable in a fluid sample of an individual, such as serum, and/or plasma. In embodiments, the biological effectors are measurable in a biological sample of an individual, such as blood plasma, wound effluent, or sputum.

As used herein, the term non-biological effector is a clinical parameter that is generally considered not to be a specific molecule. Although not a specific molecule, a non-biological effector may nonetheless still be quantifiable, either through routine measurements or through measurements that stratify the data being assessed. For example, heart rate, change in heart rate over time, respiratory rate, body temperature, blood pressure, body mass index, and other parameters would be a non-biological effector component of the risk profile. All these components are measurable or quantifiable using routine methods and equipment. Other non-biological components include data that may not be readily or routinely quantifiable or that may require a practitioner's judgment or opinion. For example, peripheral vascular disease, pulmonary hypertension, heart failure may be a quantifiable aspect of the risk profile. While there may be published guidance on classifying and diagnosing these aspects of the risk profile, assigning a numerical value to the severity, still involves observation and, to a certain extent, judgment or opinion. In some instances, the quantity or measurement assigned to a non-biological effector could be binary, e.g., “0” if absent or “1” if present. In other instances, the non-biological effector aspect of the risk profile may involve qualitative components that cannot or should not be quantified.

Levels of the clinical parameters can be assayed, detected, measured, and/or determined in a sample taken or isolated from an individual. “Sample” and “test sample” are used interchangeably herein.

Examples of test samples or sources of clinical parameters include, but are not limited to: biological fluids and/or tissues isolated from an individual or patient, which can be tested by the methods of the present application described herein, and include but are not limited to: whole blood, peripheral blood, capillary blood, serum, plasma, cerebrospinal fluid, wound effluent, urine, amniotic fluid, peritoneal fluid, pleural fluid, lymph fluids, various external secretions of the respiratory, intestinal, and genitourinary tracts, various components of exhaled breath, tears, sweat, saliva, white blood cells, tissue biopsies, and combinations thereof.

In embodiments, data quality control involves at least one of differential expression algorithms, principal component analysis, k-nearest neighbor imputation algorithms, three-sigma rule algorithms, or empirical Bayes method algorithms. Differential expression algorithms determine the fold-change from a reference sample and the p-value for the statistical difference between the sample and the reference value, which are used as decision metrics for inclusion or exclusion. Principal component analysis identifies the key variables in a multidimensional data set that explain the differences in the observations (variance) and can be used to determine if groups separate according to a priori knowledge about the samples. Nearest neighbor imputation utilizes k-nearest neighbor algorithms to predict discrete and continues values for a potential missing value. Using three-sigma rule algorithms, biomarker data (protein-based, nucleic acid-based, or metabolite-based) generated from multiplex assays may be subsetted by a variance metric, wherein a threshold for variance is set as an inclusion or exclusion criteria (e.g., only markers with a variance greater than three standard deviations will be included). Empirical Bayes method algorithms utilize the estimated distributions from the data to establish prior distributions, and are used to approximate values in a data set and subset data based on the parameters of the estimated distribution.

In embodiments, feature selection involves at least one of: unsupervised machine learning algorithm, supervised machine learning algorithm, minimum redundancy maximum relevance, Student's t-test, Mann-Whitney U test, random forest, or logistic regression. Minimum redundancy maximum relevance involves selecting features that have high correlation to the classification variable but are mathematically far away from each other. A Student's t-test utilizes the mean and variance of two distributions to generate a t-statistic and calculate the probability that the data comes from a distribution that is true under the null hypothesis. A Mann-Whitney U test is a non-parametric test that utilizes a rank-order approach to test the null hypothesis that it is equally likely that a randomly selected value from one sample will be less than or greater than a randomly selected value from a second sample. Random forest approaches include a large number (100s-10,000s) of decision trees, each of which is generated by bootstrap aggregating, where for each decision tree the discovery data is randomly sampled with replacement to generate a randomly sampled set of discovery data, and subsequently the decision tree is trained on the randomly sampled set of discovery data. In embodiments, where feature selection is performed prior to generating the random forest model, the discovery data is sampled based on the reduced set of variables from variable selection (as opposed to sampling based on all variables).

In embodiments, feature selection may involve ensemble learning methods. Ensemble methods use multiple learning algorithms to obtain better predictive performance than could be obtained from any of the constituent learning algorithms alone. The feature selection ensemble learning models include combinations of the models described herein for cluster analysis and machine learning. In embodiments, the feature selection ensemble learning models may comprise: cluster analysis, unsupervised machine learning algorithm, supervised machine learning algorithm, minimum redundancy maximum relevance, Student's t-test, Mann-Whitney U test, random forest, logistic regression, neural networks, or a combination thereof. Ensembles may also comprise: Bayes optimal classifier, classification and regression tree, bootstrap aggregating, boosting, Bayesian model averaging, Bayesian model combination, a bucket of models, stacking, or a combination thereof.

In embodiments, data may be stratified prior to feature selection. This data stratification may be achieved by using unsupervised or supervised machine learning models, including but not limited to: topological data analysis, k-means clustering, hierarchical clustering, nearest neighbor clustering, non-linear clustering (e.g., t-distributed stochastic neighbor embedding), consensus clustering, or spectral clustering.

In embodiments, systems, methods, and a non-transitory computer-readable medium of the present disclosure can execute a process by which data is aggregated about one or more individuals, machine learning algorithms perform data-mining procedures, pattern recognition, intelligent prediction, and other artificial intelligence procedures, such as for enabling prognostic or diagnostic predictions (e.g., predicting hospitalization, predicting mortality, diagnosing sepsis phenotype, detecting pathogen or pathogen class) based on clinical data (e.g., age, sex, medical history) and/or biological data (e.g., protein-based biomarkers, nucleic acid-based biomarkers, metabolite-based biomarkers, organ system function, or physiologic parameters such as heart rate). Machine learning and ensemble learning algorithms are increasingly being implemented to reveal knowledge structures to guide decisions in conditions of limited certainty, which can lead to improved decision making. This would not be possible with the use of manual techniques or traditional algorithmic approaches, because of the large number of data points involved, as well as the specific approach and data pipeline used in the analysis. However, in order to use machine learning algorithms effectively and obtain optimal results out of existing data, a machine learning engine comprising a specific sequence of approaches and feature selection implemented by machine learning or ensemble learning algorithms may be required.

Constructing such a machine learning engine and executing these machine learning or ensemble learning algorithms can improve the performance of diagnostic and prognostic prediction technologies. These improvements may include, but are not limited to increasing the accuracy, selectivity, and/or specificity of models used to perform the diagnostic or prognostic predictions. Therefore, such an engine can improve decision-making for, and delivery of treatments to, individuals and patients. While various machine learning or ensemble learning algorithms can be used for such purposes, generating a machine learning engine with desired performance characteristics can be highly domain-specific, requiring rigorous modeling, testing, and validation to select appropriate algorithms (or combinations thereof) and the parameters modeled with the algorithms to generate the machine learning system.

In embodiments, the machine learning engine may be constructed to include five major components: (1) initial data exploration, (2) data quality control, (3) stratification, (4) feature selection and outcome modeling, (5) deployment and self-improvement. It will be understood by those possessing ordinary skill in the art that these stages may not be discrete entities and there may be overlap between them, and that the output from each stage may be used to inform, calibrate, and/or improve other stages of the machine learning engine.

The initial data stage may include data preparation. Data preparation may include cleaning data (e.g., searching for outlying data, applying missing data algorithms, altering data formats), transforming data, and selecting subsets of records in case of data sets with large numbers of variables (“fields or dimensions”). The data on which data preparation is performed may be referred to as “discovery data”.

In embodiments, data preparation can include executing pre-processing operations on the data. For example, missing data may be handled through the execution of imputation algorithms that interpolate and/or estimate missing values. One example of imputation involves generating a distribution (e.g., Gaussian, Poisson, binomial, zero-inflation, beta, pert) of available data for a clinical parameter having missing data, and interpolating values for the missing data based on the distribution. Other examples of handling missing data may involve k-nearest neighbor imputation. Additionally, data may be screened for outliers and non-random variation (e.g., batch effects related to analytical platform, collection site, operator that are known or suspected a priori). Data outliers and non-random variation may be initially identified using the ‘three-sigma rule’ or principal component analysis and assessed on a case-by-case basis. Non-random variation in the data may be corrected primarily using empirical Bayesian methods. For example, the R software function “ComBat” is widely used in biomedical research to correct data sets that contain known batch effects.

In embodiments, data quality control can include reducing the dimensionality of data (e.g., protein marker data, nucleic acid marker data, metabolite marker data, clinical outcome data, administrative health data) via specific algorithms or analytic approaches. For example, host biomarkers (protein-based, nucleic acid-based, or metabolite-based) may be measured using multiplex assays that generate data on thousands of markers. Subsetting such data may be performed by conducting a differential expression analysis wherein the fold-change from a reference sample and the p-value for the statistical difference between the sample and the reference value are used as decision metrics for inclusion or exclusion. In other examples, biomarker data (protein-based, nucleic acid-based, or metabolite-based) generated from multiplex assays may be subsetted by a variance metric, wherein a threshold for variance is set as an inclusion or exclusion criteria (e.g., only markers with a variance greater than three standard deviations will be included).

In embodiments, data quality control algorithms may comprise: supervised machine learning algorithm, differential expression algorithms, principal component analysis, k-nearest neighbor imputation algorithms, three-sigma rule algorithms, or empirical Bayes method algorithms, or a combination thereof.

In embodiments, clinical parameter data can be stratified using cluster analysis algorithms, which discretize information based on measures of similarity. Thus, individuals or samples are assigned to a discrete set of groups (clusters) based on one or more shared characteristics, such as derived from the observable or measured clinical parameters. For example, these can comprise one or more host biomarkers, one or more clinical outcomes data, one or more administrative health data, or a combination thereof. A “phenotype” may thus be defined as the set of clinical parameter values that underlies a distinct cluster of individuals or samples. In embodiments, the cluster analysis algorithms may comprise: k-means clustering, hierarchical clustering, nearest neighbor clustering, non-linear clustering (e.g., t-distributed stochastic neighbor embedding), consensus clustering, or spectral clustering.

In embodiments, clinical parameter data can be stratified using topological data analysis (TDA). Unsupervised TDA approaches, such as the “Mapper” algorithm, can be used to represent highly complex data in a structured, two-dimensional network that retains the geometric “shape” (topology) of the data. Individuals or samples with a high degree of similarity, for example of host gene, protein and/or metabolite expression profiles, form groups of highly interconnected nodes which represent distinct subgroups/populations within the dataset. Unlike most “canonical” cluster analysis algorithms, TDA is able to reflect the continuous nature of many types of biological data. For example, it can capture how groups of individuals with different characteristics relate to one another, or form trends along specific axes. Groups of individuals or samples within a TDA networks can be delineated based on the persistence homology of their node density and connectivity (edges). In a similar manner to cluster analysis, a “phenotype” may thus be defined as the set of clinical parameter values that underlies a distinct TDA group of individuals or samples. Differences in the biological effectors, non-biological effectors, and/or additional metadata between phenotypes can be independently assessed for their statistical significance. Membership of a specific disease-response phenotype constitutes valuable information about an individual, and stratifying heterogeneous data sets in this manner can improve feature selection, machine learning, and predictive modelling approaches.

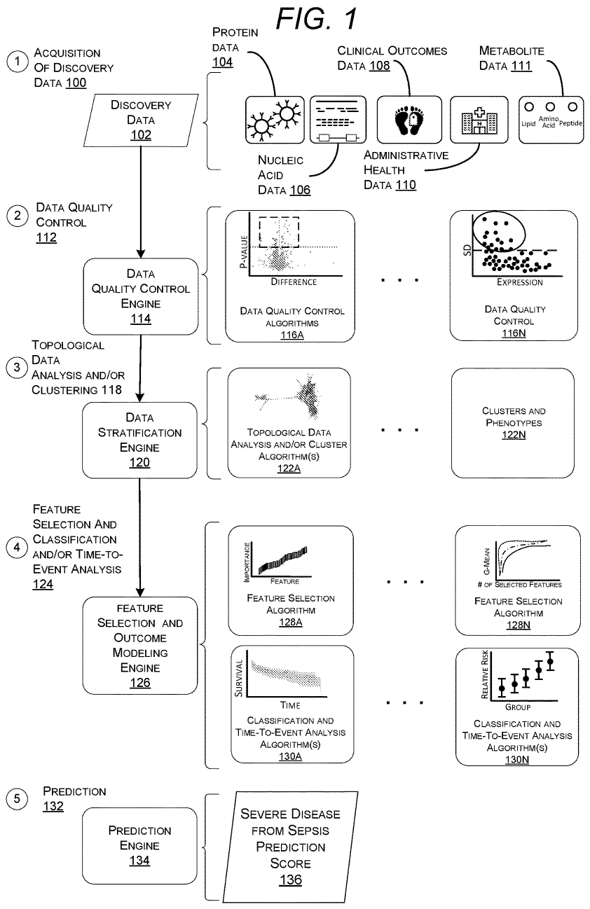

Referring now to FIG. 1, the process of predicting severe disease among individuals with sepsis or at risk of developing sepsis and its components are shown and described below. The process begins with the acquisition of discovery data 100 and executes a data quality control 112 process in a data quality control engine 114, performs topological data analysis and/or clustering 118 in a data stratification engine 120, executes feature selection and classification and/or time-to-event analyses 124 in a feature selection and outcome modeling engine 126, and the model(s) is/are deployed for prediction 132 in a prediction engine 134.

In embodiments, the discovery data 102 comprises protein data 104, nucleic acid data 106, metabolite data 111, clinical outcomes data 108, and administrative health data 110.

In embodiments, the protein data 104 may include, but are not limited to one or more of: a disintegrin and metalloproteinase with thrombospondin motifs 13 (ADAMTS13), Angiopoietin-1 (ANGPT1), Angiopoietin-2 (ANGPT2), C-C chemokine receptor ligand 2/monocyte chemoattractant protein 1 (CCL2/MCP-1), C-C chemokine receptor ligand 3/macrophage inflammatory protein 1-alpha (CCL3/MIP-1-α), C-C chemokine receptor ligand 5/regulated on activation, normal T cell expressed and secreted (CCL5/RANTES), cluster of differentiation 163 (CD163), cluster of differentiation 40 ligand (CD40L), chitinase-3-like protein 1 (CHI3L1), C-reactive protein (CRP), C-X-C motif chemokine ligand 10/interferon gamma-induced protein 10 (CXCL10/IP-10), decoy receptor 3 (Dcr3), D-dimer, E-selectin (SELE), endoglin (ENG), fas receptor (FAS), ferritins, fibrinogens, granulocyte colony-stimulating factor (G-CSF), granulocyte-macrophage colony-stimulating factor (GM-CSF), (soluble) intercellular adhesion molecule 1 (ICAM-1), interferon gamma (IFNγ), interleukin 1 beta (IL-1β), interleukin-1 receptor antagonist (IL-1RA), (soluble) interleukin-2 receptor α (IL-2Rα), interleukin-4 (IL-4), interleukin-5 (IL-5), interleukin-6 (IL-6), interleukin-6 receptor α (IL-6Rα), interleukin-7 (IL-7), interleukin-8 (IL-8), interleukin-10 (IL-10), interleukin-12 ‘p70’ (IL-12 p70), interleukin-15 (IL-15), interleukin-16 (IL-16), interleukin-17A (IL-17A), interleukin-18 (IL-18), interleukin-18-binding protein (IL-18BP), interleukin-22 (IL-22), interleukin-27 (IL-27), lipocalin-2 (LCN-2), matrix metalloproteinase-8 (MMP-8), matrix metalloproteinase-9 (MMP-9), matrix metalloproteinase-10 (MMP-10), (soluble) macrophage mannose receptors, procalcitonin (PCT), (soluble) programmed death-ligand 1 (PD-L1), pentraxin 3 (PTX3), (soluble) receptor for advanced glycation end products (RAGE), resistin (RETN), serum amyloid A proteins (SAA), tyrosine kinase with immunoglobulin-like and EGF-like domains 1 (TIE1), tyrosine kinase with immunoglobulin-like and EGF-like domains 2 (TIE2), tissue inhibitor of metalloproteinases 1 (TIMP1), tissue inhibitor of metalloproteinases 2 (TIMP2), tissue inhibitor of metalloproteinases 3 (TIMP3), tissue inhibitor of metalloproteinases 4 (TIMP4), tumor necrosis factor receptor 1 (TNF-R1), tumor necrosis factor alpha (TNFα), tissue plasminogen activator (tPA), tissue plasminogen activator inhibitor 1 (tPAI-1), TNF-related apoptosis-inducing ligand (TRAIL), (soluble) triggering receptor expressed on myeloid cells 1 (TREM1), urokinase receptor (uPar), (soluble) vascular cell adhesion molecule 1 (VCAM-1), vascular endothelial growth factors (VEGF), (soluble) vascular endothelial growth factor receptor 1 (VEGFR-1), (soluble) vascular endothelial growth factor receptor 2 (VEGFR-2), or von Willebrand factor A2 domain (vWF-A2). While these protein markers are enumerated, many more are contemplated.

In embodiments the protein markers are at least one or more of: C-reactive protein (CRP), C-X-C motif chemokine ligand 10/interferon gamma-induced protein 10 (CXCL10/IP-10), D-dimer, ferritins, fibrinogens, (soluble) intercellular adhesion molecule 1 (ICAM-1), interferon gamma (IFNγ), interleukin-1 receptor antagonist (IL-1RA), interleukin-5 (IL-5), interleukin-6 (IL-6), interleukin-6 receptor α (IL-6Rα), interleukin-18 (IL-18), interleukin-18-binding protein (IL-18BP), lipocalin-2 (LCN-2), matrix metalloproteinase-8 (MMP-8), procalcitonin (PCT), (soluble) receptor for advanced glycation end products (RAGE), tumor necrosis factor receptor 1 (TNF-R1), tumor necrosis factor alpha (TNFα), vascular endothelial growth factors (VEGF), or von Willebrand factor A2 domain (vWF-A2).

In embodiments, the nucleic acid data 106 may include, but are not limited to one or more of: adhesion G protein-coupled receptor E1 (ADGRE1), adrenoceptor β2 (ADRB2), angiotensin II receptor associated protein (AGTRAP), AKT serine/threonine kinase 1 (AKT1), 5′-aminolevulinate synthase 2 (ALAS2), alkaline phosphatase, biomineralization associated (ALPL), ankyrin repeat domain 22 (ANKRD22), annexin A3 (ANXA3), arginase 1 (ARG1), BCL2 like 1 (BCL2L1), BMX non-receptor tyrosine kinase (BMX), chromosome 6 open reading frame 62 (C6orf62), carbonic anhydrase 2 (CA2), C-C motif chemokine ligand 5 (CCL5), C-C motif chemokine receptor 3 (CCR3), CD4 molecule (CD4), CD24 molecule (CD24), CD177 molecule (CD177), CD274 molecule (CD274), cell division cycle 34, ubiqiutin conjugating enzyme (CDC34), complement factor D (CFD), chitinase 3 like 1 (CHI3L1), carbohydrate sulfotransferase 2 (CHST2), C-type lectin domain family 4 member E (CLEC4E), cytidine/uridine monophosphate kinase 2 (CMPK2), cytochrome C oxidase assembly factor 1 homolog (COA1), carnitine palmitoyltransferase 1A (CPT1A), carboxypeptidase vitellogenic like (CPVL), chondroitin sulfate N-acetylgalactosaminyltransferase 1 (CSGALNACT1), cystatin C (CST3), C-X3-C motif chemokine receptor 1 (CX3CR1), DNA damage inducible transcript 4 (DDIT4), defensin α3 (DEFA3), defensin α4 (DEFA4), DNA J heat shock protein family (Hsp40) member C1 (DNAJC1), DNA damage regulated autophagy modulator 1 (DRAM1), deoxyuridine triphosphatase (DUT), dual specificity tyrosine phosphorylation regulated kinase 3 (DYRK3), erythrocyte membrane protein band 4.2 (EPB42), family with sequence similarity 174 member C (FAM174C), F-box and WD repeat domain containing 2 (FBXW2), Fc receptor like 5 (FCRL5), ferrochelatase (FECH), fibroblast growth factor binding protein 2 (FGFBP2), Fms related receptor tyrosine kinase 3 (FLT3), formyl peptide receptor 1 (FPR1), GATA binding protein 1 (GATA1), GTPase, IMAP family member 4 (GIMAP4), GTPase, IMAP family member 7 (GIMAP7), GTPase, IMAP family member 8 (GIMAP8), G protein subunit γ2 (GNG2), granulysin (GNLY), G protein-coupled receptor 65 (GPR65), growth factor receptor bound protein 10 (GRB10), glutathione S-transferase κ1 (GSTK1), H3 histone pseudogene 6 (H3F3AP4), hemoglobin subunit α2 (HBA2), hemogen (HEMGN), HECT and RLD domain containing E3 ubiquitin protein ligase family member 6 (HERC6), H3.2 histone [putative] (HIST2H3PS2), major histocompatibility complex, class I, B (HLA-B), major histocompatibility complex, class II, DQ β1 (HLA-DQB1), high mobility group box 2 (HMGB2), 15-hydroxyprostaglandin dehydrogenase (HPGD), hydrogen voltage gated channel 1 (HVCN1), isoamyl acetate hydrolyzing esterase 1 [putative] (IAH1), intercellular adhesion molecule 1 (ICAM1), immediate early response 5 (IER5), interferon α inducible protein 6 (IFI6), interferon α inducible protein 27 (IFI27), interferon induced protein 44 (IFI44), interferon induced protein with tetratricopeptide repeats 1 (IFIT1), interferon induced protein with tetratricopeptide repeats 2 (IFIT2), interleukin 1β (IL1B), interleukin 1 receptor type 1 (IL1RA), interleukin 1 receptor type 2 (IL1R2), interleukin 10 receptor subunit α (IL10RA), interaction protein for cytohesin exchange factors 1 (IPCEF1), interferon regulatory factor 2 binding protein 2 (IRF2BP2), ISG15 ubiquitin like modifier (ISG15), JUN proto-oncogene, AP-1 transcription factor subunit (JUN), potassium voltage-gated channel subfamily E regulatory subunit 1 (KCNE1), kinesin light chain 3 (KLC3), kelch like family member 24 (KLHL24), kringle containing transmembrane protein 1 (KREMEN1), long intergenic non-protein coding RNA 861 (LIN000861), lymphocyte antigen 6 family member E (LY6E), MAPK associated protein 1 (MAPKAP1), mediator complex subunit 28 (MED28), MicroRNA 6724-4 (MIR6724-4), matrix metallopeptidase 8 (MMP8), multimerin 1 (MMRN1), myeloperoxidase (MPO), mannose receptor C type 2 (MRC2), mitochondrially encoded 12S rRNA (MT-RNR1), MX dynamin like GTPase 2 (MX2), nuclear factor, erythroid 2 like 3 (NFE2L3), 2′-5′-oligoadenylate synthetase 3 (OAS3), oleoyl-ACP hydrolase (OLAH), olfactomedin 4 (OLFM4), peptidase inhibitor 3 (PI3), phosphatidylinositol-4,5-bisphosphate 3-kinase catalytic subunit β (PIK3CB), PITH domain containing 1 (PITHD1), pyruvate kinase M1/2 (PKM), perilipin 2 (PLIN2), DNA polymerase δ interacting protein 3 (POLDIP3), RAL GTPase activating protein catalytic subunit α2 (RALGAPA2), RAN binding protein 9 (RANBP9), REST corepressor 1 (RCOR1), Rh associated glycoprotein (RHAG), RNA, U1 small nuclear 2 (RNU1-2), RNA, U1 small nuclear 4 (RNU1-4), ribosomal protein L37a (RPL37A), ribosomal protein L38 (RPL38), ribosomal protein S11 (RPS11), ribosomal protein S18 (RPS18), radical S-adenosyl methionine domain containing 2 (RSAD2), S100 calcium binding protein A8 (S100A8), S100 calcium binding protein A9 (S100A9), S100 calcium binding protein A12 (S100A12), SAM domain, SH3 domain and nuclear localization signals 1 (SAMSN1), Sin3A associated protein 30 (SAP30), strawberry notch homolog 1 (SBNO1), selenium binding protein 1 (SELENBP1), sialic acid binding Ig like lectin 10 (SIGLEC10), solute carrier family 25 member 6 (SLC25A6), solute carrier family 25 member 39 (SLC25A39), solute carrier family 39 member 8 (SLC39A8), solute carrier family 4 member 1 [Diego Blood Group] (SLC4A1), synuclein α (SNCA), small nucleolar RNA, H/ACA box 44 (SNORA44), superoxide dismutase 2 (SOD2), spectrin α, erythrocytic 1 (SPTA1), STE20 related adaptor β (STRADB), syntaxin 6 (STX6), switching B cell complex subunit SWAP70 (SWAP70), spectrin repeat containing nuclear envelope protein 2 (SYNE2), T-box transcription factor 21 (TBX21), TRAF interacting protein with forkhead associated domain (TIFA), toll like receptor 7 (TLR7), transmembrane and coiled-coil domain family 2 (TMCC2), transmembrane protein 35B (TMEM35B), transmembrane protein 273 (TMEM273), thymosin β10 (TMSB10), TNF α induced protein 6 (TNFAIP6), tyrosylprotein sulfotransferase 1 (TPST1), tripartite motif containing 4 (TRIM4), tetraspanin 5 (TSPAN5), tetratricopeptide repeat domain 9C (TTC9C), ubiquitin protein ligase E3 component N-recognin 5 (UBR5), UNC-93 homolog B1, TLR signaling regulator (UNC93B1), WASH complex subunit 2C (WASHC2C), XIAP associated factor 1 (XAF1), tyrosine 3-monooxygenase/tryptophan 5-monooxygenase activation protein E (YWHAH), and zinc finger With KRAB and SCAN domains 1 (ZKSCAN1).

In embodiments the nucleic acid markers are at least one or more of: adrenoceptor β2 (ADRB2), CD177 molecule (CD177), carboxypeptidase vitellogenic like (CPVL), C-X3-C motif chemokine receptor 1 (CX3CR1), defensin α3 (DEFA3), Fc receptor like 5 (FCRL5), G protein subunit γ2 (GNG2), interleukin 10 receptor subunit α (IL10RA), kinesin light chain 3 (KLC3), oleoyl-ACP hydrolase (OLAH), pyruvate kinase M1/2 (PKM), radical S-adenosyl methionine domain containing 2 (RSAD2), STE20 related adaptor β (STRADB), tyrosylprotein sulfotransferase 1 (TPST1), tetraspanin 5 (TSPAN5), tetratricopeptide repeat domain 9C (TTC9C), or zinc finger with KRAB and SCAN domains 1 (ZKSCAN1).

In embodiments, the metabolite data 111 may include, but are not limited to one or more of: fatty acyls and their constituent molecular species, glycerolipids and their constituent molecular species, glycerophospholipids and their constituent molecular species, sphingolipids and their constituent molecular species, sterol lipids and their constituent molecular species, prenol lipids and their constituent molecular species, saccharolipids and their constituent molecular species, polyketides and their constituent molecular species, carbohydrates and their constituent molecular species, organic acids and their derivatives and constituent molecular species, organo-heterocyclic compounds and their constituent molecular species, organo-oxygen compounds and their constituent molecular species, organo-nitrogen compounds and their constituent molecular species, amino acids and their constituent molecular species, peptides and their constituent molecular species, and nucleosides and their constituent molecular species.

In embodiments the metabolite markers are at least one or more of: carnitine, acetylcarnitine, propionylcarnitine, malonylcarnitine, methylmalonylcarnitine, hydroxypropionylcarnitine, propenoylcarnitine, butyrylcarnitine, hydroxybutyrylcarnitine, fumarylcarnitine, valerylcarnitine, glutarylcarnitine, hydroxyvalerylcarnitine, tiglylcarnitine, hexanoylcarnitine, hydroxyhexanoylcarnitine, pimeloylcarnitine, decanoylcarnitine, decadienylcarnitine, tetradecenoylcarnitine, hydroxytetradecenoylcarnitine, hydroxytetradecadienylcarnitine, hexadecanoylcarnitine, hexadecenoylcarnitine, octadecanoylcarnitine, octadecenoylcarnitine, lysophosphatidylcholine with acyl residue sum C16:0, lysophosphatidylcholine with acyl residue sum C16:1, lysophosphatidylcholine with acyl residue sum C17:0, lysophosphatidylcholine with acyl residue sum C18:0, lysophosphatidylcholine with acyl residue sum C18:1, lysophosphatidylcholine with acyl residue sum C18:2, lysophosphatidylcholine with acyl residue sum C20:3, lysophosphatidylcholine with acyl residue sum C20:4, lysophosphatidylcholine with acyl residue sum C24:0, lysophosphatidylcholine with acyl residue sum C26:0, lysophosphatidylcholine with acyl residue sum C26:1, lysophosphatidylcholine with acyl residue sum C28:0, lysophosphatidylcholine with acyl residue sum C28:1, phosphatidylcholine with diacyl residue sum C24:0, phosphatidylcholine with diacyl residue sum C28:1, phosphatidylcholine with diacyl residue sum C30:0, phosphatidylcholine with diacyl residue sum C32:0, phosphatidylcholine with diacyl residue sum C32:1, phosphatidylcholine with diacyl residue sum C32:3, phosphatidylcholine with diacyl residue sum C34:1, phosphatidylcholine with diacyl residue sum C34:2, phosphatidylcholine with diacyl residue sum C34:3, phosphatidylcholine with diacyl residue sum C34:4, phosphatidylcholine with diacyl residue sum C36:0, phosphatidylcholine with diacyl residue sum C36:1, phosphatidylcholine with diacyl residue sum C36:2, phosphatidylcholine with diacyl residue sum C36:3, phosphatidylcholine with diacyl residue sum C36:4, phosphatidylcholine with diacyl residue sum C36:5, phosphatidylcholine with diacyl residue sum C36:6, phosphatidylcholine with diacyl residue sum C38:0, phosphatidylcholine with diacyl residue sum C38:3, phosphatidylcholine with diacyl residue sum C38:4, phosphatidylcholine with diacyl residue sum C38:5, phosphatidylcholine with diacyl residue sum C38:6, phosphatidylcholine with diacyl residue sum C40:2, phosphatidylcholine with diacyl residue sum C40:3, phosphatidylcholine with diacyl residue sum C40:4, phosphatidylcholine with diacyl residue sum C40:5, phosphatidylcholine with diacyl residue sum C40:6, phosphatidylcholine with diacyl residue sum C42:0, phosphatidylcholine with diacyl residue sum C42:1, phosphatidylcholine with diacyl residue sum C42:2, phosphatidylcholine with diacyl residue sum C42:4, phosphatidylcholine with diacyl residue sum C42:5, phosphatidylcholine with diacyl residue sum C42:6, phosphatidylcholine with acyl-alkyl residue sum C30:0, phosphatidylcholine with acyl-alkyl residue sum C30:1, phosphatidylcholine with acyl-alkyl residue sum C30:2, phosphatidylcholine with acyl-alkyl residue sum C32:1, phosphatidylcholine with acyl-alkyl residue sum C32:2, phosphatidylcholine with acyl-alkyl residue sum C34:0, phosphatidylcholine with acyl-alkyl residue sum C34:1, phosphatidylcholine with acyl-alkyl residue sum C34:2, phosphatidylcholine with acyl-alkyl residue sum C34:3, phosphatidylcholine with acyl-alkyl residue sum C36:0, phosphatidylcholine with acyl-alkyl residue sum C36:1, phosphatidylcholine with acyl-alkyl residue sum C36:2, phosphatidylcholine with acyl-alkyl residue sum C36:3, phosphatidylcholine with acyl-alkyl residue sum C36:4, phosphatidylcholine with acyl-alkyl residue sum C36:5, phosphatidylcholine with acyl-alkyl residue sum C38:0, phosphatidylcholine with acyl-alkyl residue sum C38:1, phosphatidylcholine with acyl-alkyl residue sum C38:2, phosphatidylcholine with acyl-alkyl residue sum C38:3, phosphatidylcholine with acyl-alkyl residue sum C38:4, phosphatidylcholine with acyl-alkyl residue sum C38:5, phosphatidylcholine with acyl-alkyl residue sum C38:6, phosphatidylcholine with acyl-alkyl residue sum C40:1, phosphatidylcholine with acyl-alkyl residue sum C40:2, phosphatidylcholine with acyl-alkyl residue sum C40:3, phosphatidylcholine with acyl-alkyl residue sum C40:4, phosphatidylcholine with acyl-alkyl residue sum C40:5, phosphatidylcholine with acyl-alkyl residue sum C40:6, phosphatidylcholine with acyl-alkyl residue sum C42:2, phosphatidylcholine with acyl-alkyl residue sum C42:3, phosphatidylcholine with acyl-alkyl residue sum C42:5, phosphatidylcholine with acyl-alkyl residue sum C44:3, phosphatidylcholine with acyl-alkyl residue sum C44:4, phosphatidylcholine with acyl-alkyl residue sum C44:5, phosphatidylcholine with acyl-alkyl residue sum C44:6, hydroxysphingomyelin with acyl residue sum C14:1, hydroxysphingomyelin with acyl residue sum C16:1, hydroxysphingomyelin with acyl residue sum C22:1, hydroxysphingomyelin with acyl residue sum C22:2, hydroxysphingomyelin with acyl residue sum C24:1, sphingomyelin with acyl residue sum C16:0, sphingomyelin with acyl residue sum C16:1, sphingomyelin with acyl residue sum C18:0, sphingomyelin with acyl residue sum C18:1, sphingomyelin with acyl residue sum C20:2, sphingomyelin with acyl residue sum C24:0, sphingomyelin with acyl residue sum C24:1, sphingomyelin with acyl residue sum C26:0, sphingomyelin with acyl residue sum C26:1, hexoses [including glucose], alanine, arginine, asparagine, aspartate, citrulline, glutamine, glutamate, glycine, histidine, isoleucine, lysine, methionine, ornithine, phenylalanine, proline, serine, threonine, tryptophan, tyrosine, valine, asymmetric dimethylarginine, alpha-aminoadipic acid, creatinine, kynurenine, methionine sulfoxide, putrescine, sarcosine, symmetric dimethylarginine, spermidine, spermine, trans-4-hydroxyproline, or taurine.

In embodiments, the clinical outcomes data 108 may include, but are not limited to one or more of: severity or duration of symptoms, time to symptom onset or abatement, need for organ support, duration of organ support, response to treatment, admission to a hospital or intensive care unit, length of stay in a hospital or intensive care unit, mortality, time to death, duration of morbidity, incidence of long-term sequelae of infectious diseases, and re-hospitalization.

In embodiments, the administrative health data 110 may include, but are not limited to one or more of: baseline demographics, physiologic parameters, comorbid conditions including but not limited to immunocompromising conditions, past surgical history, and environmental or social exposures.

In embodiments, data quality control 112 occurs in the data quality control engine 114, which executes a series of data quality control algorithms 116A-116N (hereinafter referred to individually as “item 116A,” and generically as “item 116”) which subset data to be used in topological data analysis and/or clustering 118. The data quality control algorithms and general approach may vary depending on the characteristics of each unique data set. For example, host biomarkers (protein-based, nucleic acid-based, or metabolite-based) may be measured using multiplex assays that generate data on thousands of markers. Subsetting such data may be performed by conducting a differential expression analysis wherein the fold-change from a reference sample and the p-value for the statistical difference between the sample and the reference value are used as decision metrics for inclusion or exclusion. In other examples, biomarker data (protein-based, nucleic acid-based, or metabolite-based) generated from multiplex assays may be subsetted by a variance metric, wherein a threshold for variance is set as an inclusion or exclusion criteria (e.g., only markers with a variance greater than three standard deviations will be included). While these methods of data quality control are discussed, many more are contemplated.

In embodiments, the topological data analysis and/or clustering 118 occur in the data stratification engine 120, wherein topological data analysis and/or cluster analysis algorithms 122A-122N (hereinafter referred to individually as “item 122A,” and generically as “item 122”) are deployed upon the subsetted data from the data quality control engine 114. Cluster analysis algorithms 122 use supervised or unsupervised approaches to discretize highly complex data based on similarities in the observable or measured clinical parameters. Alternatively, topological data analysis algorithms 122, such as the “Mapper” algorithm, use unsupervised approaches to represent such data in a structured, two-dimensional network that retains the geometric ‘shape’ (topology) of the data correlations. Individuals or samples with a high degree of similarity, for example of host gene, protein and/or metabolite expression profiles, form groups of highly interconnected nodes which represent distinct subgroups/populations within the dataset. Such groups within a TDA networks can be delineated based on the persistence homology of their node density and connectivity (edges). Both cluster analysis 122 and topological data analysis 122 algorithms result in the assignment of individuals or samples to a discrete set of groups/clusters based on multiple shared characteristics, thereby enabling the definition of disease-response phenotypes. Sepsis response phenotypes can thus be defined as the profile of biomolecular, clinical, administrative health, and/or physiologic profile data of each distinct cluster. Differences in the biological effectors, non-biological effectors, and/or additional metadata between phenotypes can be assessed independently for their statistical significance. Membership of a specific sepsis response phenotype constitutes valuable information about an individual, and stratifying heterogeneous data sets in this manner can improve feature selection, machine learning, and predictive modelling approaches.

In embodiments, feature selection and classification and/or time-to-event analysis 124 occur in the feature selection and outcome modeling engine 126. Feature selection 124 involves the use of feature selection algorithms 128A-128N (hereinafter referred to individually as “item 128A,” and generically as “item 128N”) to select features (e.g., variables, parameters) for improving outcome modeling performance (as measured by model performance metrics), optimizing computational resources, removing confounders and/or mediating factors, and for temporal and/or causational interpretation. Data may be stratified in the data stratification engine 120 prior to feature selection. Alternatively, data stratification prior to feature selection may be achieved by using other unsupervised or supervised machine learning models, including but not limited to: k-means clustering, hierarchical clustering, nearest neighbor clustering, non-linear clustering (e.g., t-distributed stochastic neighbor embedding), consensus clustering, or spectral clustering. The data on which the feature selection is performed may be referred to as “discovery data”. Given that the performance of the subsequent classification and time-to-event analysis algorithms can depend strongly on the quality of the discovery data used to train the classification and time-to-event analysis algorithms, feature selection and other data preparation operations (e.g., data quality control) can be highly significant for ensuring desired performance.

In embodiments, classification and/or time-to-event analysis 124 involves the use of the classification and time-to-event analysis algorithms 130A-130N (hereinafter referred to individually as “item 130A,” and generically as “item 130N”) to calculate the prediction score for clinical outcomes in individuals with sepsis or at risk of developing sepsis (outcome modeling).

In embodiments, the prediction 132 involves the prediction of severe disease in individuals with sepsis or at risk of developing sepsis. This is performed in the prediction engine, which houses trained machine learned algorithms (e.g., trained data quality control algorithms, trained data stratification algorithms trained feature selection algorithms, trained classification and/or time-to-event analysis algorithms). The prediction engine 134 utilizes the trained machine learned algorithms to calculate and provide a clinical outcome prediction score 136 for predicting severe disease in individuals with sepsis or at risk of developing sepsis. The classification and/or time-to-event analysis algorithms 130 may include incidence rates by categorical variables or continuous variables. The classification and/or time-to-event analysis algorithms 130 may also include Kaplan-Meier estimators, Cox proportional-hazards models, cumulative incidence functions, or accelerated failure time models. While these classification and time-to-event analysis algorithms are discussed, others are contemplated.

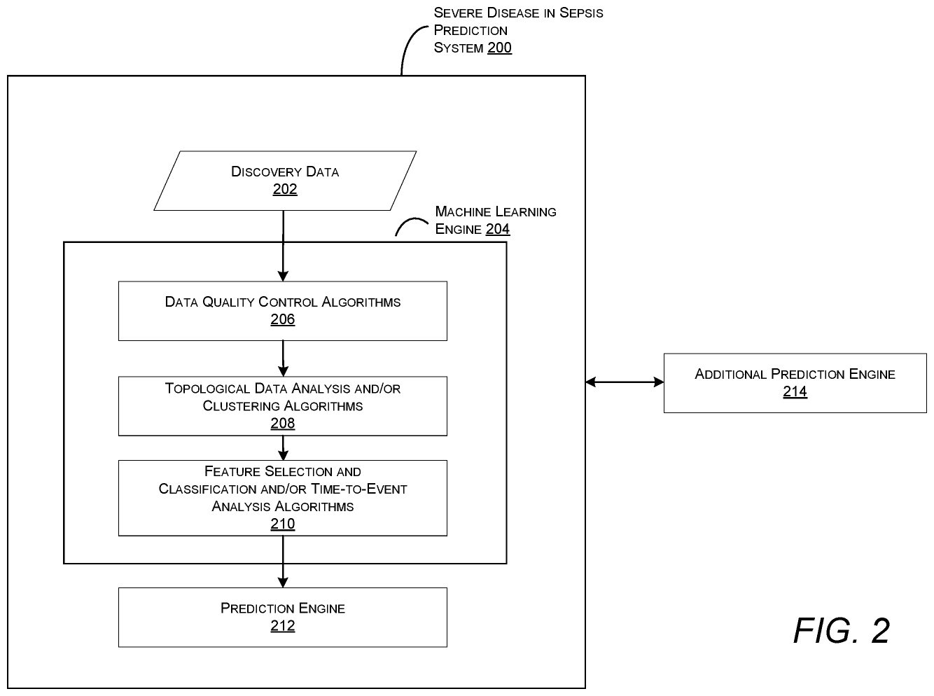

Referring to FIG. 2, in embodiments, the Severe Disease in Sepsis Prediction System 200 includes discovery data 202, a machine learning engine 204 that is comprised of data quality control algorithms 206, topological data analysis and/or clustering algorithms 208, feature selection and classification and/or time-to-event analysis algorithms 210, and a prediction engine 212. An additional prediction engine 214 is housed outside the machine learning engine but is connected to the Severe Disease in Sepsis Prediction System 200 and can feed data and models bi-directionally.

The prediction engine 212 can predict severe disease from sepsis specific to at least one second individual. The prediction engine 212 can receive, for the at least one second individual, a second value of at least one clinical parameter of the plurality of clinical parameters.

In embodiments, at least one of the received second values corresponds to a model parameter of the subset of model parameters used in the feature selection and outcome modeling engine 126. If the prediction engine 212 receives several second values of clinical parameters, of which at least one does not correspond to a model parameter of the subset of model parameters, the prediction engine 212 may execute an imputation algorithm to generate a value for such a missing parameter.

The prediction engine 212 can execute the feature selection and classification and time-to-event analysis algorithms 210 using the second value of the at least one clinical parameter to calculate the severe disease risk to the at least one second individual. In an example, the classification and time-to-event analysis algorithms 210 may include a Kaplan-Meier estimator wherein the topological data analysis and/or clustering 208 and feature selection algorithms 128 may provide a categorical variable as the predictor for the Kaplan-Meier estimator, resulting in a severe disease risk prediction and a confidence interval for each category by providing a hazard ratio for each group. In another example, a Cox Proportional-Hazards model may include the categorical variable provided from the topological data analysis and/or clustering 208, and at least one or more clinical parameters as covariates to improve the accuracy of the model, resulting in the Cox Proportional-Hazards model providing a hazard ratio for each group provided by the topological data analysis and/or clustering 208, as well as the confidence intervals for the categorical variable and each of the covariates. As such, the prediction engine 212 can output a prediction that the second individual will experience severe disease from sepsis based on the overall probabilities (e.g., based on a ratio of the overall probabilities).

In embodiments, the additional prediction engine 214 may be house outside the Severe Disease in Sepsis Prediction System 200 and may contained machine learned models, but is connected to the Severe Disease in Sepsis Prediction System 200 and may feed data and models bi-directionally.

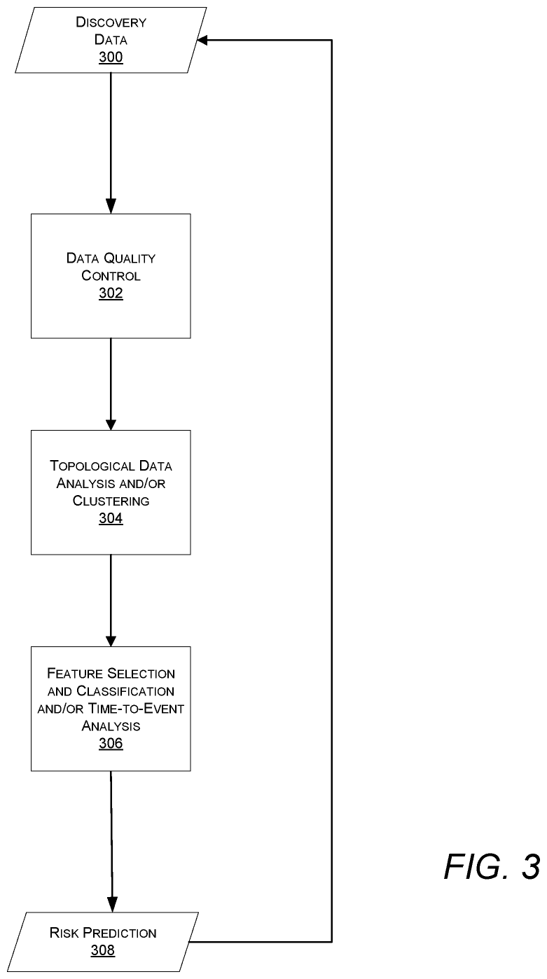

Referring now to FIG. 3, a process for predicting severe disease in individuals with sepsis or at risk of developing sepsis, and the flow of data that occurs in the machine learning engine 204 is described. The process can be performed by various systems described herein, including the Severe Disease in Sepsis Prediction System 200 and/or the remote device 436. The discovery data 300 comprises protein data 104, nucleic acid data 106, metabolite data 111, clinical outcomes data 108, and administrative health data 110.

In embodiments, preprocessing is executed on the discovery data 300. Pre-processing may be performed before data quality control 302 and/or topological data analysis and/or clustering 304 are performed on the data. In embodiments, an imputation algorithm can be executed to generate values for missing data in the discovery data 300. In embodiments, at least one of up-sampling or predictor rank transformations is executed on the data of the discovery database. Up-sampling and/or predictor rank transformation can be executed only for variable selection to accommodate class imbalance and non-normality in the data. While up-sampling or predictor rank transformations are discussed, many others are contemplated.

At data quality control 302 the dimensionality of data may be reduced via specific algorithms or analytic approaches. For example, protein data 104, nucleic acid data 106 and/or metabolite data 111 may be generated using multiplex assays that generate data on thousands of markers. Subsetting such data may be performed by conducting a differential expression analysis wherein the fold-change from a reference sample and the p-value for the statistical difference between the sample and the reference value are used as decision metrics for inclusion or exclusion. In other examples, biomarker data (protein-based, nucleic acid-based, or metabolite-based) generated from multiplex assays may be subsetted by a variance metric, wherein a threshold for variance is set as an inclusion or exclusion criteria (e.g., only markers with a variance greater than three standard deviations will be included). While these methods of data quality control are discussed, many more are contemplated.

At topological data analysis and/or clustering 304, the cluster analysis discretizes highly complex data based on similarities in the plurality of subsets of clinical parameters. Alternatively, the topological data analysis groups individuals or samples based on similarities in the plurality of subsets of clinical parameters, as well as the algebraic topology of the same data, and clusters are delineated based on persistence homology of the node density and connectivity. Sepsis response phenotypes are then defined based on the identified clusters using either approach.

At feature selection and classification and/or time-to-event analysis 306, one or more feature selection machine learning or ensemble learning models, and classification and/or time-to event analysis algorithms are executed. The subsets of model parameters are selected from the plurality of clinical parameters of the discovery data 300, such that a count of each subset of model parameters is less than a count of the clinical parameters. Feature selection machine learning engines such as constraint-based algorithms, constrain-based structure learning algorithms, and/or constraint-based local discovery learning algorithms can be used to select the subsets of model parameters. For example, the machine learning engine 204 can execute machine learning algorithms such as minimum redundancy maximum relevance, Student's t-test, Mann-Whitney U test, random forest, and logistic regression. In embodiments, the clinical parameters are randomly re-ordered prior to feature selection. In embodiments, data may be stratified in the data stratification engine 120 prior to feature selection. Alternatively, data stratification prior to feature selection may be achieved by using other unsupervised or supervised machine learning models, including but not limited to topological data analysis, k-means clustering, hierarchical clustering, nearest neighbor clustering, non-linear cluster (e.g., t-distributed Stochastic neighbor embedding), consensus clustering, or spectral clustering.

For classification analyses, one or more models and/or algorithms that are designed to classify the probability that a given individual or given sample belongs to a particular group may be used. For example, at feature selection and classification 306, the machine learning engine 204 can execute a regression model, a pattern recognition algorithm, a decision tree, or other machine learning algorithm to calculate a risk, risk ratio, odds, odds ratio, or other probability output. While these models and/or algorithms are discussed, others are contemplated.

For time-to-event analyses, one or more models and/or algorithms that are designed to forecast or predict duration of time until one or more events (e.g., death of a biological organism) may be used. For example, at feature selection and time-to-event analysis 306, the machine learning engine 204 can execute a log-rank test, a Kaplan-Meier function, a survival function, a hazard function, a Cox Proportional-Hazards regression, survival trees, survival random forests, or calculate life tables. While these models and/or algorithms are discussed, others are contemplated.

At risk prediction 308 second values of clinical parameters are received. The second values may be received for at least one second individual. In embodiments, at least one of the received second values corresponds to a model parameter of the subset of model parameters used in the classification and/or time-to-event analysis machine learning algorithm 306. If several second values of clinical parameters are received, of which at least one does not correspond to a model parameter of the subset of model parameters, an imputation algorithm may be executed to generate a value for such a missing parameter. The candidate classification machine learning is executed using the corresponding subset of model parameters and the second value of the at least one clinical parameter to calculate the prediction of the clinical outcome specific to the at least one second individual. The predicted outcome specific to the at least one second individual is outputted. For example, the predicted outcome may be displayed on an electronic device to a user or may be provided as an audio output. The predicted outcome may be transmitted to another device. The predicted outcome may include at least one of an indication that the second individual has sepsis, that the second individual is likely to have sepsis (e.g., relative to a confidence threshold), or that the second individual has an increased risk for experiencing severe disease from sepsis relative to a reference risk level.

In embodiments, there are provided methods for predicting severe disease in an individual with sepsis and/or assessing risk factors (e.g., clinical parameters) in an individual, the methods comprising, consisting of, or consisting essentially of measuring, assessing, detecting, assaying, and/or determining one or more clinical parameters, such as one or more selected from level of the following in a sample from the individual: adhesion G protein-coupled receptor E1 (ADGRE1), adrenoceptor β2 (ADRB2), angiotensin II receptor associated protein (AGTRAP), AKT serine/threonine kinase 1 (AKT1), 5′-aminolevulinate synthase 2 (ALAS2), alkaline phosphatase, biomineralization associated (ALPL), ankyrin repeat domain 22 (ANKRD22), annexin A3 (ANXA3), arginase 1 (ARG1), BCL2 like 1 (BCL2L1), BMX non-receptor tyrosine kinase (BMX), chromosome 6 open reading frame 62 (C6orf62), carbonic anhydrase 2 (CA2), C-C motif chemokine ligand 5 (CCL5), C-C motif chemokine receptor 3 (CCR3), CD4 molecule (CD4), CD24 molecule (CD24), CD177 molecule (CD177), CD274 molecule (CD274), cell division cycle 34, ubiqiutin conjugating enzyme (CDC34), complement factor D (CFD), chitinase 3 like 1 (CHI3L1), carbohydrate sulfotransferase 2 (CHST2), C-type lectin domain family 4 member E (CLEC4E), cytidine/uridine monophosphate kinase 2 (CMPK2), cytochrome C oxidase assembly factor 1 homolog (COA1), carnitine palmitoyltransferase 1A (CPT1A), carboxypeptidase vitellogenic like (CPVL), chondroitin sulfate N-acetylgalactosaminyltransferase 1 (CSGALNACT1), cystatin C (CST3), C-X3-C motif chemokine receptor 1 (CX3CR1), DNA damage inducible transcript 4 (DDIT4), defensin α3 (DEFA3), defensin α4 (DEFA4), DNA J heat shock protein family (Hsp40) member C1 (DNAJC1), DNA damage regulated autophagy modulator 1 (DRAM1), deoxyuridine triphosphatase (DUT), dual specificity tyrosine phosphorylation regulated kinase 3 (DYRK3), erythrocyte membrane protein band 4.2 (EPB42), family with sequence similarity 174 member C (FAM174C), F-box and WD repeat domain containing 2 (FBXW2), Fc receptor like 5 (FCRL5), ferrochelatase (FECH), fibroblast growth factor binding protein 2 (FGFBP2), Fms related receptor tyrosine kinase 3 (FLT3), formyl peptide receptor 1 (FPR1), GATA binding protein 1 (GATA1), GTPase, IMAP family member 4 (GIMAP4), GTPase, IMAP family member 7 (GIMAP7), GTPase, IMAP family member 8 (GIMAP8), G protein subunit γ2 (GNG2), granulysin (GNLY), G protein-coupled receptor 65 (GPR65), growth factor receptor bound protein 10 (GRB10), glutathione S-transferase κ1 (GSTK1), H3 histone pseudogene 6 (H3F3AP4), hemoglobin subunit α2 (HBA2), hemogen (HEMGN), HECT and RLD domain containing E3 ubiquitin protein ligase family member 6 (HERC6), H3.2 histone [putative](HIST2H3PS2), major histocompatibility complex, class I, B (HLA-B), major histocompatibility complex, class II, DQ β1 (HLA-DQB1), high mobility group box 2 (HMGB2), 15-hydroxyprostaglandin dehydrogenase (HPGD), hydrogen voltage gated channel 1 (HVCN1), isoamyl acetate hydrolyzing esterase 1 [putative] (IAH1), intercellular adhesion molecule 1 (ICAM1), immediate early response 5 (IER5), interferon α inducible protein 6 (IFI6), interferon α inducible protein 27 (IFI27), interferon induced protein 44 (IFI44), interferon induced protein with tetratricopeptide repeats 1 (IFIT1), interferon induced protein with tetratricopeptide repeats 2 (IFIT2), interleukin 1β (IL1B), interleukin 1 receptor type 1 (IL1RA), interleukin 1 receptor type 2 (IL1 R2), interleukin 10 receptor subunit α (IL10RA), interaction protein for cytohesin exchange factors 1 (IPCEF1), interferon regulatory factor 2 binding protein 2 (IRF2BP2), ISG15 ubiquitin like modifier (ISG15), JUN proto-oncogene, AP-1 transcription factor subunit (JUN), potassium voltage-gated channel subfamily E regulatory subunit 1 (KCNE1), kinesin light chain 3 (KLC3), kelch like family member 24 (KLHL24), kringle containing transmembrane protein 1 (KREMEN1), long intergenic non-protein coding RNA 861 (LINC00861), lymphocyte antigen 6 family member E (LY6E), MAPK associated protein 1 (MAPKAP1), mediator complex subunit 28 (MED28), MicroRNA 6724-4 (MIR6724-4), matrix metallopeptidase 8 (MMP8), multimerin 1 (MMRN1), myeloperoxidase (MPO), mannose receptor C type 2 (MRC2), mitochondrially encoded 12S rRNA (MT-RNR1), MX dynamin like GTPase 2 (MX2), nuclear factor, erythroid 2 like 3 (NFE2L3), 2′-5′-oligoadenylate synthetase 3 (OAS3), oleoyl-ACP hydrolase (OLAH), olfactomedin 4 (OLFM4), peptidase inhibitor 3 (PI3), phosphatidylinositol-4,5-bisphosphate 3-kinase catalytic subunit β (PIK3CB), PITH domain containing 1 (PITHD1), pyruvate kinase M1/2 (PKM), perilipin 2 (PLIN2), DNA polymerase δ interacting protein 3 (POLDIP3), RAL GTPase activating protein catalytic subunit α2 (RALGAPA2), RAN binding protein 9 (RANBP9), REST corepressor 1 (RCOR1), Rh associated glycoprotein (RHAG), RNA, U1 small nuclear 2 (RNU1-2), RNA, U1 small nuclear 4 (RNU1-4), ribosomal protein L37a (RPL37A), ribosomal protein L38 (RPL38), ribosomal protein S11 (RPS11), ribosomal protein S18 (RPS18), radical S-adenosyl methionine domain containing 2 (RSAD2), S100 calcium binding protein A8 (S100A8), S100 calcium binding protein A9 (S100A9), S100 calcium binding protein A12 (S100A12), SAM domain, SH3 domain and nuclear localization signals 1 (SAMSN1), Sin3A associated protein 30 (SAP30), strawberry notch homolog 1 (SBNO1), selenium binding protein 1 (SELENBP1), sialic acid binding Ig like lectin 10 (SIGLEC10), solute carrier family 25 member 6 (SLC25A6), solute carrier family 25 member 39 (SLC25A39), solute carrier family 39 member 8 (SLC39A8), solute carrier family 4 member 1 [Diego Blood Group] (SLC4A1), synuclein α (SNCA), small nucleolar RNA, H/ACA box 44 (SNORA44), superoxide dismutase 2 (SOD2), spectrin α, erythrocytic 1 (SPTA1), STE20 related adaptor β (STRADB), syntaxin 6 (STX6), switching B cell complex subunit SWAP70 (SWAP70), spectrin repeat containing nuclear envelope protein 2 (SYNE2), T-box transcription factor 21 (TBX21), TRAF interacting protein with forkhead associated domain (TIFA), toll like receptor 7 (TLR7), transmembrane and coiled-coil domain family 2 (TMCC2), transmembrane protein 35B (TMEM35B), transmembrane protein 273 (TMEM273), thymosin β10 (TMSB10), TNF α induced protein 6 (TNFAIP6), tyrosylprotein sulfotransferase 1 (TPST1), tripartite motif containing 4 (TRIM4), tetraspanin 5 (TSPAN5), tetratricopeptide repeat domain 9C (TTC9C), ubiquitin protein ligase E3 component N-recognin 5 (UBR5), UNC-93 homolog B1, TLR signaling regulator (UNC93B1), WASH complex subunit 2C (WASHC2C), XIAP associated factor 1 (XAF1), tyrosine 3-monooxygenase/tryptophan 5-monooxygenase activation protein E (YWHAH), and zinc finger With KRAB and SCAN domains 1 (ZKSCAN1), a disintegrin and metalloproteinase with thrombospondin motifs 13 (ADAMTS13), Angiopoietin-1 (ANGPT1), Angiopoietin-2 (ANGPT2), C-C chemokine receptor ligand 2/monocyte chemoattractant protein 1 (CCL2/MCP-1), C-C chemokine receptor ligand 3/macrophage inflammatory protein 1-alpha (CCL3/MIP-1-α), C-C chemokine receptor ligand 5/regulated on activation, normal T cell expressed and secreted (CCL5/RANTES), cluster of differentiation 163 (CD163), cluster of differentiation 40 ligand (CD40L), chitinase-3-like protein 1 (CHI3L1), C-reactive protein (CRP), C-X-C motif chemokine ligand 10/interferon gamma-induced protein 10 (CXCL10/IP-10), decoy receptor 3 (Dcr3), D-dimer, E-selectin (SELE), endoglin (ENG), fas receptor (FAS), ferritins, fibrinogens, granulocyte colony-stimulating factor (G-CSF), granulocyte-macrophage colony-stimulating factor (GM-CSF), (soluble) intercellular adhesion molecule 1 (ICAM-1), interferon gamma (IFNγ), interleukin 1 beta (IL-1β), interleukin-1 receptor antagonist (IL-1RA), (soluble) interleukin-2 receptor α (IL-2Rα), interleukin-4 (IL-4), interleukin-5 (IL-5), interleukin-6 (IL-6), interleukin-6 receptor α (IL-6Rα), interleukin-7 (IL-7), interleukin-8 (IL-8), interleukin-10 (IL-10), interleukin-12 ‘p70’ (IL-12 p70), interleukin-15 (IL-15), interleukin-16 (IL-16), interleukin-17A (IL-17A), interleukin-18 (IL-18), interleukin-18-binding protein (IL-18BP), interleukin-22 (IL-22), interleukin-27 (IL-27), lipocalin-2 (LCN-2), matrix metalloproteinase-8 (MMP-8), matrix metalloproteinase-9 (MMP-9), matrix metalloproteinase-10 (MMP-10), (soluble) macrophage mannose receptors, procalcitonin (PCT), (soluble) programmed death-ligand 1 (PD-L1), pentraxin 3 (PTX3), (soluble) receptor for advanced glycation end products (RAGE), resistin (RETN), serum amyloid A proteins (SAA), tyrosine kinase with immunoglobulin-like and EGF-like domains 1 (TIE1), tyrosine kinase with immunoglobulin-like and EGF-like domains 2 (TIE2), tissue inhibitor of metalloproteinases 1 (TIMP1), tissue inhibitor of metalloproteinases 2 (TIMP2), tissue inhibitor of metalloproteinases 3 (TIMP3), tissue inhibitor of metalloproteinases 4 (TIMP4), tumor necrosis factor receptor 1 (TNF-R1), tumor necrosis factor alpha (TNFα), tissue plasminogen activator (tPA), tissue plasminogen activator inhibitor 1 (tPAI-1), TNF-related apoptosis-inducing ligand (TRAIL), (soluble) triggering receptor expressed on myeloid cells 1 (TREM1), urokinase receptor (uPar), (soluble) vascular cell adhesion molecule 1 (VCAM-1), vascular endothelial growth factors (VEGF), (soluble) vascular endothelial growth factor receptor 1 (VEGFR-1), (soluble) vascular endothelial growth factor receptor 2 (VEGFR-2), von Willebrand factor A2 domain (vWF-A2), fatty acyls and their constituent molecular species, glycerolipids and their constituent molecular species, glycerophospholipids and their constituent molecular species, sphingolipids and their constituent molecular species, sterol lipids and their constituent molecular species, prenol lipids and their constituent molecular species, saccharolipids and their constituent molecular species, polyketides and their constituent molecular species, carbohydrates and their constituent molecular species, organic acids and their derivatives and constituent molecular species, organo-heterocyclic compounds and their constituent molecular species, organo-oxygen compounds and their constituent molecular species, organo-nitrogen compounds and their constituent molecular species, amino acids and their constituent molecular species, peptides and their constituent molecular species, nucleosides and their constituent molecular species, severity or duration of symptoms, time to symptom onset or abatement, need for organ support, duration of organ support, response to treatment, admission to a hospital or intensive care unit, length of stay in a hospital or intensive care unit, mortality, time to death, duration of morbidity, incidence of long-term sequelae of infectious diseases, re-hospitalization, baseline demographics, physiologic parameters, comorbid conditions including but not limited to immunocompromising conditions, past surgical history, or environmental or social exposures.

In embodiments, one or more clinical parameters, two or more clinical parameters, three or more clinical parameters, four or more clinical parameters, five or more clinical parameters, six or more clinical parameters, seven or more clinical parameters, eight or more clinical parameters, nine or more clinical parameters, ten or more clinical parameters, 11 or more clinical parameters, 12 or more clinical parameters, 13 or more clinical parameters, 14 or more clinical parameters, 15 or more clinical parameters, 16 or more clinical parameters, 17 or more clinical parameters, 18 or more clinical parameters, 19 or more clinical parameters, 20 or more clinical parameters, 21 or more clinical parameters, 22 or more clinical parameters, 23 or more clinical parameters, 24 or more clinical parameters, 25 or more clinical parameters, 26 or more clinical parameters, 27 or more clinical parameters, 28 or more clinical parameters, 29 or more clinical parameters, 30 or more clinical parameters, 31 or more clinical parameters, 32 or more clinical parameters, 33 or more clinical parameters, 34 or more clinical parameters, 35 or more clinical parameters, 36 or more clinical parameters, 37 or more clinical parameters, 38 or more clinical parameters, 39 or more clinical parameters, 40 or more clinical parameters, 41 or more clinical parameters, 42 or more clinical parameters, 43 or more clinical parameters, 44 or more clinical parameters, 45 or more clinical parameters, such as selected from those set forth above are measured, assessed, detected, assayed, and/or determined. In embodiments, 2, 3, 4, 5, 6, 7, or 8 clinical parameters are measured, assessed, detected, assayed, and/or determined.

To assay, detect, measure, and/or determine levels of individual clinical parameters, one or more samples is taken or isolated from the individual. In embodiments, at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 samples are taken or isolated from the individual. The one or more samples may or may not be processed prior assaying levels of the factors, risk factors, biomarkers, clinical parameters, and/or components. For example, whole blood may be taken from an individual and the blood sample may be processed, e.g., centrifuged, to isolate plasma or serum from the blood. The one or more samples may or may not be stored, e.g., frozen, prior to processing or analysis.

In embodiments, levels of individual biomarkers in a sample isolated from an individual are assessed, detected, measured, and/or determined using one or more biological methods, such as but not limited to: ELISA assays; Western Blot; multiplexed immunoassays; quantitative arrays; PCR; RNA sequencing; DNA sequencing; Northern Blot analysis; Luminex proteomic data; RNA-seq; transcriptomic data; quantitative polymerase chain reaction (qPCR) data; microarray, mass spectrometry (MS); MS in conjunction with liquid chromatography (LC), gas chromatography (GC), or supercritical fluid chromatography (SFC); or quantitative bacteriology data.

In embodiments, the biomarkers include nucleic acids, proteins, and metabolites isolated from biological samples, for example tissue, organ, exhaled breath, or biological fluids of an individual. Examples of biological fluids include: whole blood, serum, plasma, sweat, urine, saliva, sputum, peritoneal fluid, wound effluent, and spinal fluid.

To determine levels of clinical parameters, particularly biomarkers, it is not necessary that an entire biomarker molecule, e.g., a full-length protein or an entire RNAtranscript, be present or fully sequenced. In other words, determining levels of, for example, a fragment of protein being analyzed may be sufficient to conclude or assess that an individual component of the risk profile being analyzed is increased or decreased. Similarly, if, for example, arrays or blots are used to determine biomarker levels, the presence, absence, and/or strength of a detectable signal may be sufficient to assess levels of biomarkers.

In embodiments, clinical parameters are detected, measured, assayed, assessed, and/or determined in a sample isolated from the individual at different time points, such as before, at a first time point after, and/or at a subsequent time point after the individual has an exposure, injury, wound, or condition that puts them at risk of severe disease from sepsis, such as having a viral or bacterial infection, undergoing a medical surgical or dental procedure, having an open wound or trauma, undergoing hemodialysis, or undergoing an organ transplant procedure. For example, embodiments of the methods described herein may comprise detecting biomarkers at two, three, four, five, six, seven, eight, nine, 10 or even more time points over a period of time, such as a week or more, two weeks or more, three weeks or more, four weeks or more, a month or more, two months or more, three months or more, four months or more, five months or more, six months or more, seven months or more, eight months or more, nine months or more, ten months or more, 11 months or more, a year or more or even two years or longer. The methods also include embodiments in which the individual is assessed before and/or during and/or after treatment for sepsis. In embodiments, the methods are useful for monitoring the efficacy of treatment of sepsis, and comprise detecting clinical parameters, such as biomarkers in a sample isolated from the individual, at least one, two, three, four, five, six, seven, eight, nine or 10 or more different time points prior to beginning treatment for sepsis and subsequently detecting clinical parameters, such as at least one, two, three, four, five, six, seven, eight, nine or 10 or more different time points after beginning of treatment for sepsis, and determining the changes, if any, in the levels detected.

In embodiments there are provided methods of determining a risk profile for severe disease in individuals with sepsis or at risk of developing sepsis, wherein the risk of severe disease consists essentially of one or more components based on one or more clinical parameters selected from the following: adhesion G protein-coupled receptor E1 (ADGRE1), adrenoceptor β2 (ADRB2), angiotensin II receptor associated protein (AGTRAP), AKT serine/threonine kinase 1 (AKT1), 5′-aminolevulinate synthase 2 (ALAS2), alkaline phosphatase, biomineralization associated (ALPL), ankyrin repeat domain 22 (ANKRD22), annexin A3 (ANXA3), arginase 1 (ARG1), BCL2 like 1 (BCL2L1), BMX non-receptor tyrosine kinase (BMX), chromosome 6 open reading frame 62 (C6orf62), carbonic anhydrase 2 (CA2), C-C motif chemokine ligand 5 (CCL5), C-C motif chemokine receptor 3 (CCR3), CD4 molecule (CD4), CD24 molecule (CD24), CD177 molecule (CD177), CD274 molecule (CD274), cell division cycle 34, ubiqiutin conjugating enzyme (CDC34), complement factor D (CFD), chitinase 3 like 1 (CHI3L1), carbohydrate sulfotransferase 2 (CHST2), C-type lectin domain family 4 member E (CLEC4E), cytidine/uridine monophosphate kinase 2 (CMPK2), cytochrome C oxidase assembly factor 1 homolog (COA1), carnitine palmitoyltransferase 1A (CPT1A), carboxypeptidase vitellogenic like (CPVL), chondroitin sulfate N-acetylgalactosaminyltransferase 1 (CSGALNACT1), cystatin C (CST3), C-X3-C motif chemokine receptor 1 (CX3CR1), DNA damage inducible transcript 4 (DDIT4), defensin α3 (DEFA3), defensin α4 (DEFA4), DNA J heat shock protein family (Hsp40) member C1 (DNAJC1), DNA damage regulated autophagy modulator 1 (DRAM1), deoxyuridine triphosphatase (DUT), dual specificity tyrosine phosphorylation regulated kinase 3 (DYRK3), erythrocyte membrane protein band 4.2 (EPB42), family with sequence similarity 174 member C (FAM174C), F-box and WD repeat domain containing 2 (FBXW2), Fc receptor like 5 (FCRL5), ferrochelatase (FECH), fibroblast growth factor binding protein 2 (FGFBP2), Fms related receptor tyrosine kinase 3 (FLT3), formyl peptide receptor 1 (FPR1), GATA binding protein 1 (GATA1), GTPase, IMAP family member 4 (GIMAP4), GTPase, IMAP family member 7 (GIMAP7), GTPase, IMAP family member 8 (GIMAP8), G protein subunit γ2 (GNG2), granulysin (GNLY), G protein-coupled receptor 65 (GPR65), growth factor receptor bound protein 10 (GRB10), glutathione S-transferase κ1 (GSTK1), H3 histone pseudogene 6 (H3F3AP4), hemoglobin subunit α2 (HBA2), hemogen (HEMGN), HECT and RLD domain containing E3 ubiquitin protein ligase family member 6 (HERC6), H3.2 histone [putative] (HIST2H3PS2), major histocompatibility complex, class I, B (HLA-B), major histocompatibility complex, class II, DQ β1 (HLA-DQB1), high mobility group box 2 (HMGB2), 15-hydroxyprostaglandin dehydrogenase (HPGD), hydrogen voltage gated channel 1 (HVCN1), isoamyl acetate hydrolyzing esterase 1 [putative] (IAH1), intercellular adhesion molecule 1 (ICAM1), immediate early response 5 (IER5), interferon α inducible protein 6 (IFI6), interferon α inducible protein 27 (IFI27), interferon induced protein 44 (IFI44), interferon induced protein with tetratricopeptide repeats 1 (IFIT1), interferon induced protein with tetratricopeptide repeats 2 (IFIT2), interleukin 1β (IL1B), interleukin 1 receptor type 1 (IL1RA), interleukin 1 receptor type 2 (IL1R2), interleukin 10 receptor subunit α (IL10RA), interaction protein for cytohesin exchange factors 1 (IPCEF1), interferon regulatory factor 2 binding protein 2 (IRF2BP2), ISG15 ubiquitin like modifier (ISG15), JUN proto-oncogene, AP-1 transcription factor subunit (JUN), potassium voltage-gated channel subfamily E regulatory subunit 1 (KCNE1), kinesin light chain 3 (KLC3), kelch like family member 24 (KLHL24), kringle containing transmembrane protein 1 (KREMEN1), long intergenic non-protein coding RNA 861 (LINC00861), lymphocyte antigen 6 family member E (LY6E), MAPK associated protein 1 (MAPKAP1), mediator complex subunit 28 (MED28), MicroRNA 6724-4 (MIR6724-4), matrix metallopeptidase 8 (MMP8), multimerin 1 (MMRN1), myeloperoxidase (MPO), mannose receptor C type 2 (MRC2), mitochondrially encoded 12S rRNA (MT-RNR1), MX dynamin like GTPase 2 (MX2), nuclear factor, erythroid 2 like 3 (NFE2L3), 2′-5′-oligoadenylate synthetase 3 (OAS3), oleoyl-ACP hydrolase (OLAH), olfactomedin 4 (OLFM4), peptidase inhibitor 3 (PI3), phosphatidylinositol-4,5-bisphosphate 3-kinase catalytic subunit β (PIK3CB), PITH domain containing 1 (PITHD1), pyruvate kinase M1/2 (PKM), perilipin 2 (PLIN2), DNA polymerase δ interacting protein 3 (POLDIP3), RAL GTPase activating protein catalytic subunit α2 (RALGAPA2), RAN binding protein 9 (RANBP9), REST corepressor 1 (RCOR1), Rh associated glycoprotein (RHAG), RNA, U1 small nuclear 2 (RNU1-2), RNA, U1 small nuclear 4 (RNU1-4), ribosomal protein L37a (RPL37A), ribosomal protein L38 (RPL38), ribosomal protein S11 (RPS11), ribosomal protein S18 (RPS18), radical S-adenosyl methionine domain containing 2 (RSAD2), S100 calcium binding protein A8 (S100A8), S100 calcium binding protein A9 (S100A9), S100 calcium binding protein A12 (S100A12), SAM domain, SH3 domain and nuclear localization signals 1 (SAMSN1), Sin3A associated protein 30 (SAP30), strawberry notch homolog 1 (SBNO1), selenium binding protein 1 (SELENBP1), sialic acid binding Ig like lectin 10 (SIGLEC10), solute carrier family 25 member 6 (SLC25A6), solute carrier family 25 member 39 (SLC25A39), solute carrier family 39 member 8 (SLC39A8), solute carrier family 4 member 1 [Diego Blood Group] (SLC4A1), synuclein α (SNCA), small nucleolar RNA, H/ACA box 44 (SNORA44), superoxide dismutase 2 (SOD2), spectrin α, erythrocytic 1 (SPTA1), STE20 related adaptor β (STRADB), syntaxin 6 (STX6), switching B cell complex subunit SWAP70 (SWAP70), spectrin repeat containing nuclear envelope protein 2 (SYNE2), T-box transcription factor 21 (TBX21), TRAF interacting protein with forkhead associated domain (TIFA), toll like receptor 7 (TLR7), transmembrane and coiled-coil domain family 2 (TMCC2), transmembrane protein 35B (TMEM35B), transmembrane protein 273 (TMEM273), thymosin β10 (TMSB10), TNF α induced protein 6 (TNFAIP6), tyrosylprotein sulfotransferase 1 (TPST1), tripartite motif containing 4 (TRIM4), tetraspanin 5 (TSPAN5), tetratricopeptide repeat domain 9C (TTC9C), ubiquitin protein ligase E3 component N-recognin 5 (UBR5), UNC-93 homolog B1, TLR signaling regulator (UNC93B1), WASH complex subunit 2C (WASHC2C), XIAP associated factor 1 (XAF1), tyrosine 3-monooxygenase/tryptophan 5-monooxygenase activation protein E (YWHAH), and zinc finger With KRAB and SCAN domains 1 (ZKSCAN1), a disintegrin and metalloproteinase with thrombospondin motifs 13 (ADAMTS13), Angiopoietin-1 (ANGPT1), Angiopoietin-2 (ANGPT2), C-C chemokine receptor ligand 2/monocyte chemoattractant protein 1 (CCL2/MCP-1), C-C chemokine receptor ligand 3/macrophage inflammatory protein 1-alpha (CCL3/MIP-1-α), C-C chemokine receptor ligand 5/regulated on activation, normal T cell expressed and secreted (CCL5/RANTES), cluster of differentiation 163 (CD163), cluster of differentiation 40 ligand (CD40L), chitinase-3-like protein 1 (CHI3L1), C-reactive protein (CRP), C-X-C motif chemokine ligand 10/interferon gamma-induced protein 10 (CXCL10/IP-10), decoy receptor 3 (Dcr3), D-dimer, E-selectin (SELE), endoglin (ENG), fas receptor (FAS), ferritins, fibrinogens, granulocyte colony-stimulating factor (G-CSF), granulocyte-macrophage colony-stimulating factor (GM-CSF), (soluble) intercellular adhesion molecule 1 (ICAM-1), interferon gamma (IFNγ), interleukin 1 beta (IL-1β), interleukin-1 receptor antagonist (IL-1RA), (soluble) interleukin-2 receptor α (IL-2Rα), interleukin-4 (IL-4), interleukin-5 (IL-5), interleukin-6 (IL-6), interleukin-6 receptor α (IL-6Rα), interleukin-7 (IL-7), interleukin-8 (IL-8), interleukin-10 (IL-10), interleukin-12 ‘p70’ (IL-12 p70), interleukin-15 (IL-15), interleukin-16 (IL-16), interleukin-17A (IL-17A), interleukin-18 (IL-18), interleukin-18-binding protein (IL-18BP), interleukin-22 (IL-22), interleukin-27 (IL-27), lipocalin-2 (LCN-2), matrix metalloproteinase-8 (MMP-8), matrix metalloproteinase-9 (MMP-9), matrix metalloproteinase-10 (MMP-10), (soluble) macrophage mannose receptors, procalcitonin (PCT), (soluble) programmed death-ligand 1 (PD-L1), pentraxin 3 (PTX3), (soluble) receptor for advanced glycation end products (RAGE), resistin (RETN), serum amyloid A proteins (SAA), tyrosine kinase with immunoglobulin-like and EGF-like domains 1 (TIE1), tyrosine kinase with immunoglobulin-like and EGF-like domains 2 (TIE2), tissue inhibitor of metalloproteinases 1 (TIMP1), tissue inhibitor of metalloproteinases 2 (TIMP2), tissue inhibitor of metalloproteinases 3 (TIMP3), tissue inhibitor of metalloproteinases 4 (TIMP4), tumor necrosis factor receptor 1 (TNF-R1), tumor necrosis factor alpha (TNFα), tissue plasminogen activator (tPA), tissue plasminogen activator inhibitor 1 (tPAI-1), TNF-related apoptosis-inducing ligand (TRAIL), (soluble) triggering receptor expressed on myeloid cells 1 (TREM1), urokinase receptor (uPar), (soluble) vascular cell adhesion molecule 1 (VCAM-1), vascular endothelial growth factors (VEGF), (soluble) vascular endothelial growth factor receptor 1 (VEGFR-1), (soluble) vascular endothelial growth factor receptor 2 (VEGFR-2), von Willebrand factor A2 domain (vWF-A2), fatty acyls and their constituent molecular species, glycerolipids and their constituent molecular species, glycerophospholipids and their constituent molecular species, sphingolipids and their constituent molecular species, sterol lipids and their constituent molecular species, prenol lipids and their constituent molecular species, saccharolipids and their constituent molecular species, polyketides and their constituent molecular species, carbohydrates and their constituent molecular species, organic acids and their derivatives and constituent molecular species, organo-heterocyclic compounds and their constituent molecular species, organo-oxygen compounds and their constituent molecular species, organo-nitrogen compounds and their constituent molecular species, amino acids and their constituent molecular species, peptides and their constituent molecular species, nucleosides and their constituent molecular species, severity or duration of symptoms, time to symptom onset or abatement, need for organ support, duration of organ support, response to treatment, admission to a hospital or intensive care unit, length of stay in a hospital or intensive care unit, mortality, time to death, duration of morbidity, incidence of long-term sequelae of infectious diseases, re-hospitalization, baseline demographics, physiologic parameters, comorbid conditions including but not limited to immunocompromising conditions, past surgical history, or environmental or social exposures.

In embodiments, the risk of severe disease in an individual with sepsis or at risk of developing sepsis is calculated from one or more clinical parameters, two or more clinical parameters, three or more clinical parameters, four or more clinical parameters, five or more clinical parameters, six or more clinical parameters, seven or more clinical parameters, eight or more clinical parameters, nine or more clinical parameters, ten or more clinical parameters, 11 or more clinical parameters, 12 or more clinical parameters, 13 or more clinical parameters, 14 or more clinical parameters, 15 or more clinical parameters, 16 or more clinical parameters, 17 or more clinical parameters, 18 or more clinical parameters, 19 or more clinical parameters, 20 or more clinical parameters, 21 or more clinical parameters, 22 or more clinical parameters, 23 or more clinical parameters, 24 or more clinical parameters, 25 or more clinical parameters, 26 or more clinical parameters, 27 or more clinical parameters, 28 or more clinical parameters, 29 or more clinical parameters, 30 or more clinical parameters, 31 or more clinical parameters, 32 or more clinical parameters, 33 or more clinical parameters, 34 or more clinical parameters, 35 or more clinical parameters, 36 or more clinical parameters, 37 or more clinical parameters, 38 or more clinical parameters, 39 or more clinical parameters, 40 or more clinical parameters, 41 or more clinical parameters, 42 or more clinical parameters, 43 or more clinical parameters, 44 or more clinical parameters, 45 or more clinical parameters, such as selected from those set forth above. In embodiments, the risk of severe disease in an individual with sepsis or at risk of developing sepsis is calculated from 2, 3, 4, 5, 6, 7, or 8 clinical parameters such as selected from those set forth above. In embodiments, an individual is diagnosed as having an increased risk experiencing severe disease from sepsis if the individual's five, four, three, two or even one of the components or factors herein are at abnormal levels. It should be understood that individual levels of risk factor need not be correlated with increased risk in order for the risk profile value to indicate that the individual has an increased risk of experiencing severe disease from sepsis.

In embodiments, one or more clinical parameters are detected in a sample from the individual that is a biological fluid or tissue isolated from the individual. Biological fluids or tissues include but are not limited to: whole blood, peripheral blood, capillary blood, serum, plasma, cerebrospinal fluid, wound effluent, urine, amniotic fluid, peritoneal fluid, pleural fluid, lymph fluids, various external secretions of the respiratory, intestinal, and genitourinary tracts, various components of exhaled breath, tears, sweat, saliva, white blood cells, and tissue biopsies.

In embodiments, the measurements of the individual components themselves are used in the risk profile for severe disease in an individual with sepsis or at risk of developing sepsis, and these levels can be used to provide a “binary” value to each component, e.g., “elevated” or “not elevated.” Each of the binary values can be converted to a number, e.g., “1” or “0,” respectively.

In embodiments, the “risk of severe disease in an individual with sepsis or at risk of developing sepsis” can be a single value, number, factor or score given as an overall collective value to the individual components of the profile. For example, if each component is assigned a value, such as above, the component value may simply be the overall score of each individual or categorical value. For example, if a single categorical variable is used as the basis of the risk profile for predicting severe disease, then a hazard ratio of 2.5 might be used to convey a 250% increased risk of severe disease compared to a reference group. In this manner, the “risk of severe disease in an individual with sepsis or at risk of developing sepsis value” could be a useful single number or score, the actual value or magnitude of which could be an indication of the actual risk of severe disease, e.g., the “more positive” the value, the greater the risk of severe disease.

In embodiments, the “risk of severe disease in an individual with sepsis or at risk of developing sepsis value” can be a series of values, numbers, factors or scores given to the individual components of the overall profile. In another embodiment, the “risk of severe disease in an individual with sepsis or at risk of developing sepsis value” may be a combination of values, numbers, factors or scores given to individual components of the profile as well as values, numbers, factors or scores collectively given to a group of components, such as a host biomarker portion. In another example, the risk profile value may comprise or consist of individual values, number or scores for specific component as well as values, numbers or scores for a group of components.

In embodiments, individual values from the “risk of severe disease in an individual with sepsis or at risk of developing sepsis profile” can be used to develop a single score, such as a “combined risk index,” which may utilize weighted scores from the individual component values reduced to a diagnostic number value. The combined risk index may also be generated using non-weighted scores from the individual component values. In embodiments, when the “combined risk index” exceeds a specific threshold level, such as may be determined by a range of values developed similarly from a population of one or more control (normal) subjects, the individual may be deemed to have a high risk, or higher than normal risk, of experiencing severe disease from sepsis, whereas maintaining a normal range value of the “combined risk index” would indicate a low or minimal risk of severe disease. In these embodiments, the threshold value may be set by the combined risk index from a population of one or more control (normal) subjects.

In embodiments, the value of the “risk of severe disease in an individual with sepsis or at risk of developing sepsis profile” can be the collection of data from the individual measurements, and need not be converted to a scoring system, such that the “risk profile value” is a collection of the individual measurements of the individual components of the profile.

In embodiments, the individual's “risk of severe disease in an individual with sepsis or at risk of developing sepsis profile” is compared to a reference “risk of severe disease in an individual with sepsis or at risk of developing sepsis profile”. In embodiments, the reference “risk of severe disease in an individual with sepsis or at risk of developing sepsis profile” value is calculated from clinical parameters previously detected for the individual. Thus, the present application also includes methods of monitoring the progression of sepsis toward severe disease in an individual, with the methods comprising determining the individual's risk profile at more than one-time point. For example, embodiments of the methods of the present application will comprise determining the individual's “risk of severe disease in an individual with sepsis or at risk of developing sepsis profile” at two, three, four, five, six, seven, eight, nine, 10 or even more time points over a period of time, such as a week or more, two weeks or more, three weeks or more, four weeks or more, a month or more, two months or more, three months or more, four months or more, five months or more, six months or more, seven months or more, eight months or more, nine months or more, ten months or more, 11 months or more, a year or more or even two years or longer. The methods described herein also include embodiments in which the individual's risk profile is assessed before and/or during and/or after treatment of sepsis. In other words, the present application also includes methods of monitoring the efficacy of treatment of sepsis by assessing the individual's “risk of severe disease in an individual with sepsis or at risk of developing sepsis profile” over the course of the treatment and after the treatment. In embodiments, the methods of monitoring the efficacy of treatment of sepsis comprise determining the individual's “risk of severe disease in an individual with sepsis or at risk of developing sepsis profile” at least one, two, three, four, five, six, seven, eight, nine or 10 or more different time points prior to the receipt of treatment for sepsis and subsequently determining the individual's “risk of severe disease in an individual with sepsis or at risk of developing sepsis profile” at least one, two, three, four, five, six, seven, eight, nine or 10 or more different time points after beginning of treatment for sepsis, and determining the changes, if any, in the “risk of severe disease in an individual with sepsis or at risk of developing sepsis profile” of the individual. The treatment may be any treatment designed to cure, remove or diminish the symptoms and/or cause(s) of sepsis.

In embodiments, the reference “risk of severe disease in an individual with sepsis or at risk of developing sepsis profile” value is calculated from clinical parameters detected for a population of one or more reference subjects when the reference subjects did not have detectable signs that would put them at risk for severe disease. In embodiments, the reference “risk of severe disease in an individual with sepsis or at risk of developing sepsis profile” value is calculated from clinical parameters detected for a population of reference subjects having an exposure, injury, wound, or condition that puts them at risk of developing sepsis and severe disease from sepsis, such as an infection.

The levels or values of the clinical parameters compared to reference levels can vary. In embodiments, the levels or values of any one or more of the factors, risk factors, biomarkers, clinical parameters, and/or components is at least 1.05, 1.1, 1.2, 1.3, 1.4, 1.5, 1.6, 1.7, 1.8, 1.9, 2, 3, 4, 5, 6, 7, 8, 9, 10, 11, 12, 13, 14, 15, 16, 17, 18, 19, 20, 30, 40, 50, 60, 70, 80, 90, 100, 500, 1,000, or 10,000-fold higher than reference levels or values. In embodiments, the levels or values of any one or more of the factors, risk factors, biomarkers, clinical parameters, and/or components is at least 1.05, 1.1, 1.2, 1.3, 1.4, 1.5, 1.6, 1.7, 1.8, 1.9, 2, 3, 4, 5, 6, 7, 8, 9, 10, 11, 12, 13, 14, 15, 16, 17, 18, 19, 20, 30, 40, 50, 60, 70, 80, 90, 100, 500, 1,000, or 10,000-fold lower than reference levels or values. In the alternative, the levels or values of the factors or components may be normalized to a standard and these normalized levels or values can then be compared to one another to determine if a factor or component is lower, higher or about the same.

In embodiments, an increase in the individual's “risk of severe disease in an individual with sepsis or at risk of developing sepsis profile” value as compared to a reference “risk of severe disease in an individual with sepsis or at risk of developing sepsis profile” value indicates that the individual has an increased risk of severe disease from sepsis.

In embodiments, the individual's “risk of severe disease in an individual with sepsis or at risk of developing sepsis profile” is compared to the profile that is deemed to be a “normal” “risk of severe disease in an individual with sepsis or at risk of developing sepsis profile”. To establish a “normal” “risk of severe disease in an individual with sepsis or at risk of developing sepsis profile”, an individual or group of individuals may be first assessed to ensure they have no signs, symptoms or diagnostic indicators that they may experience severe disease from sepsis. Then, the “risk of severe disease in an individual with sepsis profile” of the individual or group of individuals can be determined to establish a “normal risk of severe disease in an individual with sepsis or at risk of developing sepsis profile.” In embodiments, a “normal risk of severe disease in an individual with sepsis or at risk of developing sepsis profile” can be ascertained from the same individual when the individual is deemed healthy, such as when the individual does not have an exposure, injury, wound, or condition that puts the individual at risk of experiencing severe disease from sepsis. In embodiments, however, a “risk of severe disease in an individual with sepsis or at risk of developing sepsis profile” from a “normal individual,” e.g., a “normal risk of severe disease in an individual with sepsis or at risk of developing sepsis profile,” is from an individual who has sepsis but does not have any concurrent conditions that may increase the risk of severe disease. Thus, in embodiments, a “normal” “risk of severe disease in an individual with sepsis or at risk of developing sepsis profile” is assessed in the same individual from whom the sample is taken, prior to the onset of any signs, symptoms or diagnostic indicators that they may experience severe disease from sepsis. For example, the “normal risk of severe disease in an individual with sepsis or at risk of developing sepsis profile” may be assessed in a longitudinal manner based on data regarding the individual at an earlier point in time, enabling a comparison between the “risk of severe disease in an individual with sepsis or at risk of developing sepsis profile” (and values thereof) over time.

In embodiments, a “normal risk of severe disease in an individual with sepsis or at risk of developing sepsis profile” is assessed in a sample from a different individual (from the individual being analyzed) and this different individual does not have, or is not suspected of, experiencing severe disease from sepsis. In embodiments, the “normal risk of severe disease in an individual with sepsis or at risk of developing sepsis profile” is assessed in a population of healthy individuals, the constituents of which display no signs, symptoms or diagnostic indicators that they may have sepsis. Thus, the individual's “risk of severe disease in an individual with sepsis or at risk of developing sepsis profile” can be compared to a normal risk profile generated from a single normal sample or a risk profile generated from more than one normal sample.

In embodiments, such as for univariate analysis, e.g., a Wilcoxon rank-sum test can be used to identify which biomarkers from specific patient groups are associated with a specific indication, outcome, or specific phenotype. The assessment of the levels of the individual components of the “risk of severe disease in an individual with sepsis or at risk of developing sepsis profile” can be expressed as absolute or relative values and may or may not be expressed in relation to another component, a standard, an internal standard or another molecule or compound known to be in the sample. If the levels are assessed as relative to a standard or internal standard, the standard or internal standard may be added to the test sample prior to, during or after sample processing.

The present disclosure also describes arrays including biomarkers comprising proteins nucleic acids, or metabolites described herein for predicting severe disease of an individual with sepsis or at risk of developing sepsis. In embodiments, proteins and nucleic acids can be linked to chips, such as microarray chips (see U.S. Pat. Nos. 6,040,138 and 7,148,058). Binding to proteins or nucleic acids on arrays can be detected by scanning the microarray with a variety of laser or charge coupled device (CCD)-based scanners, and extracting features with software packages, for example, Imagene (Biodiscovery, Hawthorne, Calif.), Feature Extraction Software (Agilent), Scanalyze (Eisen, M. 1999. SCANALYZE User Manual; Stanford Univ., Stanford, Calif. Ver 2.32.), or GenePix (Axon Instruments). An array panel including one or more biomarkers for severe disease in an individual with sepsis can be used for predicting the risk of an individual for experiencing a specified clinical outcome and/or for monitoring a patient undergoing treatment for sepsis. In embodiments, the array is a microarray.

In addition to arrays, other approaches to measure nucleic acids or proteins may be used. For example, RNA-sequencing techniques may be used, which may include single cell RNA-sequencing, direct RNA-sequencing, and/or next-gen RNA-sequencing. In addition to the approaches to measure nucleic acids or proteins, methods to measure metabolites may be used. For example, these techniques may include mass spectrometry, gas chromatography, liquid chromatography, supercritical fluid chromatography, or capillary electrophoresis, or a combination thereof.

In embodiments, the arrays described herein can be used to predict severe disease of an individual with sepsis or at risk of developing sepsis. The arrays can be used to predict mortality of an individual with sepsis. The method includes using the arrays to detect or obtain the levels of one or more biomarkers described herein. The method can also include comparing the results of an array to a respective control for predict severe disease of an individual with sepsis or at risk of developing sepsis. The respective control can be an array for a normal individual.

In embodiments, the methods described herein include predicting predict severe disease of an individual with sepsis or at risk of developing sepsis comprising detecting and/or measuring one or more biomarkers described herein. The method can include comparing the results of the detection and/or measured level of one or more biomarkers to a respective control. The respective control can include markers of a normal individual.

As will be appreciated by one skilled in the art, aspects of the present disclosure may be embodied as a system, method or computer program product. Accordingly, aspects of the present disclosure may take the form of an entirely hardware embodiment, an entirely software embodiment (including firmware, resident software, micro-code, etc.) or an embodiment combining software and hardware aspects that may all generally be referred to herein as a “circuit,” “engine,” “module,” or “system.” Furthermore, aspects of the present disclosure may take the form of a computer program product embodied in one or more computer readable medium(s) having computer readable program code embodied thereon. Aspects of the present disclosure may be implemented using one or more analog and/or digital electrical or electronic components, and may include a microprocessor, a microcontroller, an application-specific integrated circuit (ASIC), a field programmable gate array (FPGA), programmable logic and/or other analog and/or digital circuit elements configured to perform various input/output, control, analysis and other functions described herein, such as by executing instructions of a computer program product.

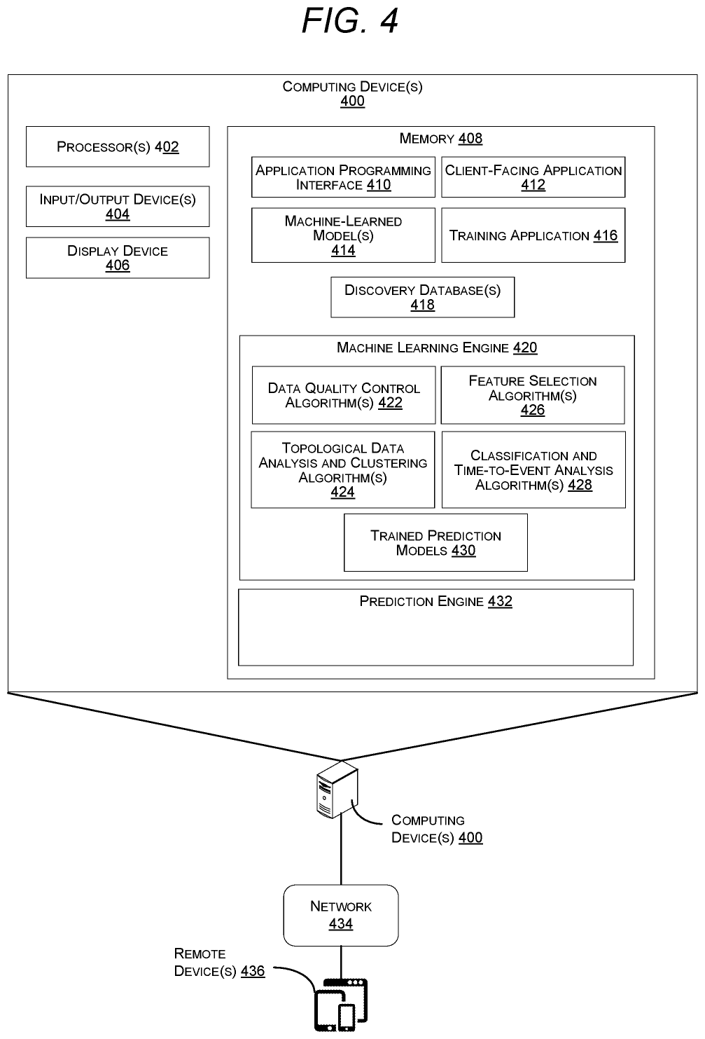

In embodiments, the computer device, computer readable media, network, and remote device may be arranged in the architecture depicted in FIG. 4. The computing device 400 houses at least, but is not limited to: a processor(s) 402, input/output device(s) 404, a display device 406, memory 408, a machine learning engine 420, and a prediction engine 432. The memory includes at least, but is not limited to an application programming interface 410, a client-facing application 412, machine learned models 414, training application 416, a discovery database 418, and a machine learning engine 420 that comprises data quality control algorithms 422, topological data analysis and clustering algorithms 424, feature selection algorithms 426, classification and time-to-event analysis algorithms 428, and trained prediction models 430. The memory also includes a prediction engine 432.

In embodiments, the computing device(s) can be accessed through a network 434 by a remote device 436. The network enables communication via internet with a secure and protected host website operating the machine learning engine and prediction engine and providing an output after predictive variables are entered.

In embodiments, the remote device 436 can be connected to the network using any number or combination of communication standards (e.g., Bluetooth, GSM, CDMA, TDNM, WCDMA, OFDM, GPRS, EV-DO, Wi-Fi, WiMAX, S02.xx, UWB, LTE, satellite). The connections may also be through wired communication features, such as USB ports, serial ports, IEEE 1394 ports, optical ports, parallel ports, and/or any other suitable wired communication port.

In embodiments, the input/output device(s) 404 may include one or more of: a computer, a keyboard, a mouse, a mobile device (e.g., a mobile phone, a tablet, a laptop), a screen, a microphone, or a printing device. The user input device can include various user interface elements such as keys, buttons, sliders, knobs, touchpads (e.g., resistive or capacitive touchpads), or microphones. In embodiments, the user interface device includes a touchscreen display device and user input device, such that the user interface device can receive user inputs as touch inputs and determine commands indicated by the user inputs based on detecting location, intensity, duration, or other parameters of the touch inputs.

In embodiments, the application programming interface 410 and the client-facing application 412 may be implemented using various software environments, including but not limited to: SAS and R software packages. SAS (“Statistical Analysis Software”) is a general-purpose package. Ready-to-use procedures are available in SAS; these procedures handle a wide range of statistical analyses, including but not limited to: analysis of variance, regression, categorical data analysis, multivariate analysis, survival analysis, cluster analysis, and nonparametric analysis. R is a free, general purpose, open-source software package that complies with and runs on a variety of UNIX platforms. There are many additional packages that run within the R general purpose software package, including topological data analysis, cluster analysis, and machine learning. While these are discussed, many other statistical and or machine learning software packages are contemplated.

Any combination of one or more computer readable medium(s) may be utilized to store the machine-learned models 414, the training application 416, and the discovery database 418. The one or more computer readable medium(s) may also be utilized to store the machine learning engine 420 and the data quality control algorithms 422, the topological data analysis and clustering algorithms 424, the feature selection algorithms 426, and the classification and time-to-event analysis algorithms 428. Additionally, the trained prediction models 430 may be stored in the machine learning engine 420. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium may be transmitted using any appropriate medium, including but not limited to wireless, wireline, optical fiber cable, RF, etc., or any suitable combination of the foregoing. Computer program code for carrying out operations for aspects of the present disclosure may be written in any combination of one or more programming languages, including an object-oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Aspects of the present disclosure are described herein with reference to flowchart illustrations and/or block diagrams of methods, apparatus (systems) and computer program products according to embodiments of the disclosure. It will be understood that each block of the flowchart illustrations and/or block diagrams, and combinations of blocks in the flowchart illustrations and/or block diagrams, can be implemented by computer program instructions. These computer program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks. The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

The flowchart and block diagrams in the Figures illustrate the architecture, functionality, and operation of possible implementations of systems, methods and computer program products according to embodiments of the present disclosure. In this regard, each block in the flowchart or block diagrams may represent a module, segment, or portion of code, which comprises one or more executable instructions for implementing the specified logical function(s). It should also be noted that, in some alternative implementations, the functions noted in the blocks may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

Although the figures show a specific order of method steps, the order of the steps may differ from what is depicted. Also, two or more steps may be performed concurrently or with partial concurrence. Such variation will depend on the software and hardware systems chosen and on designer choice. All such variations are within the scope of the disclosure. Likewise, software implementations could be accomplished with standard programming techniques with rule-based logic and other logic to accomplish the various connection steps, processing steps, comparison steps and decision steps.

As will be understood by one of ordinary skill in the art, each embodiment disclosed herein can comprise, consist essentially of, or consist of its particular stated element, step, ingredient or component. Thus, the terms “include” or “including” should be interpreted to recite: “comprise, consist of, or consist essentially of.” The transition term “comprise” or “comprises” means includes, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment.

In addition, unless otherwise indicated, numbers expressing quantities of ingredients, constituents, reaction conditions and so forth used in the specification and claims are to be understood as being modified by the term “about”. Accordingly, unless indicated to the contrary, the numerical parameters set forth in the specification and attached claims are approximations that may vary depending upon the desired properties sought to be obtained by the subject matter presented herein. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the subject matter presented herein are approximations, the numerical values set forth in the specific examples are reported as precisely as possible. Any numerical values, however, inherently contain certain errors necessarily resulting from the standard deviation found in their respective testing measurements.

When further clarity is required, the term “about” has the meaning reasonably ascribed to it by a person skilled in the art when used in conjunction with a stated numerical value or range, i.e., denoting somewhat more or somewhat less than the stated value or range, to within a range of ±20% of the stated value; 15% of the stated value; 10% of the stated value; 5% of the stated value; ±4% of the stated value; 3% of the stated value; 2% of the stated value; 1% of the stated value; or ±any percentage between 1% and 20% of the stated value.

The use of any and all examples, or exemplary language (e.g., “such as”) provided herein is intended merely to better illuminate the invention and does not pose a limitation on the scope of the subject matter otherwise claimed. No language in the specification should be construed as indicating any non-claimed element essential to the practice of the invention.

The following examples illustrate exemplary methods provided herein. These examples are not intended, nor are they to be construed, as limiting the scope of the disclosure. It will be clear that the methods can be practiced otherwise than as particularly described herein. Numerous modifications and variations are possible in view of the teachings herein and, therefore, are within the scope of the disclosure.

### Exemplary Embodiments

1. A method of generating a model predicting severe disease in an individual with sepsis or at risk of developing sepsis comprising: generating a discovery database storing first values of a plurality of clinical parameters and clinical outcomes associated with a plurality of first subjects; executing a plurality of data quality control algorithms to select a subset of clinical parameters from the plurality of clinical parameters; executing topological data analysis and/or clustering for the plurality of subsets of clinical parameters; executing a plurality of feature selection machine learning and/or ensemble learning models based on a plurality of classification and/or time-to-event analysis algorithms; and outputting a model for predicting severe disease in the individual with sepsis or at risk of developing sepsis.
2. The method of embodiment 1, further comprising pre-processing data that is stored in the discovery database, including: determining that a first value of at least one of the plurality of clinical parameters is missing; estimating a reference value for the at least one of the plurality of clinical parameters that is missing; and storing the reference value as the first value of the at least one of the plurality of clinical parameters in the discovery database.
3. The method of embodiment 1 or 2, wherein the plurality of data quality control algorithms comprise at least one of: differential expression algorithms, principal component analysis, k-nearest neighbor imputation algorithms, three-sigma rule algorithms, and empirical Bayes method algorithms.
4. The method of any one of embodiments 1-3, wherein the topological data analysis groups individuals or samples based on similarities in the plurality of subsets of clinical parameters, as well as the algebraic topology of the same data, wherein clusters are delineated based on the persistence homology of the node density and connectivity, and wherein sepsis response phenotypes are defined based on the identified clusters.
5. The method of any one of embodiments 1-4, wherein the cluster analysis discretizes the plurality of subsets of clinical parameters based on measures of similarity, wherein clusters are delineated based on the persistence homology of the node density and connectivity, and wherein sepsis response phenotypes are defined based on the identified clusters.
6. The method of any one of embodiments 1-5, wherein the feature selection machine learning models comprise at least one of: unsupervised machine learning algorithm, supervised machine learning algorithm, minimum redundancy maximum relevance, Student's t-test, Mann-Whitney U test, random forest, logistic regression, or neural networks.
7. The method of any one of embodiments 1-6, wherein the feature selection ensemble learning models comprise at least one of: cluster analysis, unsupervised machine learning algorithm, supervised machine learning algorithm, minimum redundancy maximum relevance, Student's t-test, Mann-Whitney U test, random forest, logistic regression, neural networks, Bayes optimal classifier, classification and regression tree, bootstrap aggregating, boosting, Bayesian model averaging, Bayesian model combination, a bucket of models, or stacking.
8. The method of any one of embodiments 1-7, wherein the plurality of clinical parameters comprise one or more nucleic acid data markers, one or more protein data markers, one or more metabolite data markers, one or more clinical outcomes data, one or more administrative health data, or a combination thereof.
9. The method of embodiment 8, wherein the nucleic acid data markers comprise one or more of: level of adhesion G protein-coupled receptor E1 (ADGRE1) in a sample from the individual, level of adrenoceptor β2 (ADRB2) in a sample from the individual, level of angiotensin II receptor associated protein (AGTRAP) in a sample from the individual, level of AKT serine/threonine kinase 1 (AKT1) in a sample from the individual, level of 5′-aminolevulinate synthase 2 (ALAS2) in a sample from the individual, level of alkaline phosphatase, biomineralization associated (ALPL) in a sample from the individual, level of ankyrin repeat domain 22 (ANKRD22) in a sample from the individual, level of annexin A3 (ANXA3) in a sample from the individual, level of arginase 1 (ARG1) in a sample from the individual, level of BCL2 like 1 (BCL2L1) in a sample from the individual, level of BMX non-receptor tyrosine kinase (BMX) in a sample from the individual, level of chromosome 6 open reading frame 62 (C6orf62) in a sample from the individual, level of carbonic anhydrase 2 (CA2) in a sample from the individual, level of C-C motif chemokine ligand 5 (CCL5) in a sample from the individual, level of C-C motif chemokine receptor 3 (CCR3) in a sample from the individual, level of CD4 molecule (CD4) in a sample from the individual, level of CD24 molecule (CD24) in a sample from the individual, level of CD177 molecule (CD177) in a sample from the individual, level of CD274 molecule (CD274) in a sample from the individual, level of cell division cycle 34, ubiqiutin conjugating enzyme (CDC34) in a sample from the individual, level of complement factor D (CFD) in a sample from the individual, level of chitinase 3 like 1 (CHI3L1) in a sample from the individual, level of carbohydrate sulfotransferase 2 (CHST2) in a sample from the individual, level of C-type lectin domain family 4 member E (CLEC4E) in a sample from the individual, level of cytidine/uridine monophosphate kinase 2 (CMPK2) in a sample from the individual, level of cytochrome C oxidase assembly factor 1 homolog (COA1) in a sample from the individual, level of carnitine palmitoyltransferase 1A (CPT1A) in a sample from the individual, level of carboxypeptidase vitellogenic like (CPVL) in a sample from the individual, level of chondroitin sulfate N-acetylgalactosaminyltransferase 1 (CSGALNACT1) in a sample from the individual, level of cystatin C (CST3) in a sample from the individual, level of C-X3-C motif chemokine receptor 1 (CX3CR1) in a sample from the individual, level of DNA damage inducible transcript 4 (DDIT4) in a sample from the individual, level of defensin α3 (DEFA3) in a sample from the individual, level of defensin α4 (DEFA4) in a sample from the individual, level of DNA J heat shock protein family (Hsp40) member C1 (DNAJC1) in a sample from the individual, level of DNA damage regulated autophagy modulator 1 (DRAM1) in a sample from the individual, level of deoxyuridine triphosphatase (DUT) in a sample from the individual, level of dual specificity tyrosine phosphorylation regulated kinase 3 (DYRK3) in a sample from the individual, level of erythrocyte membrane protein band 4.2 (EPB42) in a sample from the individual, level of family with sequence similarity 174 member C (FAM174C) in a sample from the individual, level of F-box and WD repeat domain containing 2 (FBXW2) in a sample from the individual, level of Fc receptor like 5 (FCRL5) in a sample from the individual, level of ferrochelatase (FECH) in a sample from the individual, level of fibroblast growth factor binding protein 2 (FGFBP2) in a sample from the individual, level of Fms related receptor tyrosine kinase 3 (FLT3) in a sample from the individual, level of formyl peptide receptor 1 (FPR1) in a sample from the individual, level of GATA binding protein 1 (GATA1) in a sample from the individual, level of GTPase, IMAP family member 4 (GIMAP4) in a sample from the individual, level of GTPase, IMAP family member 7 (GIMAP7) in a sample from the individual, level of GTPase, IMAP family member 8 (GIMAP8) in a sample from the individual, level of G protein subunit γ2 (GNG2) in a sample from the individual, level of granulysin (GNLY) in a sample from the individual, level of G protein-coupled receptor 65 (GPR65) in a sample from the individual, level of growth factor receptor bound protein 10 (GRB10) in a sample from the individual, level of glutathione S-transferase κ1 (GSTK1) in a sample from the individual, level of H3 histone pseudogene 6 (H3F3AP4) in a sample from the individual, level of hemoglobin subunit α2 (HBA2) in a sample from the individual, level of hemogen (HEMGN) in a sample from the individual, level of HECT and RLD domain containing E3 ubiquitin protein ligase family member 6 (HERC6) in a sample from the individual, level of H3.2 histone [putative] (HIST2H3PS2) in a sample from the individual, level of major histocompatibility complex, class I, B (HLA-B) in a sample from the individual, level of major histocompatibility complex, class II, DQ β1 (HLA-DQB1) in a sample from the individual, level of high mobility group box 2 (HMGB2) in a sample from the individual, level of 15-hydroxyprostaglandin dehydrogenase (HPGD) in a sample from the individual, level of hydrogen voltage gated channel 1 (HVCN1) in a sample from the individual, level of isoamyl acetate hydrolyzing esterase 1 [putative] (IAH1) in a sample from the individual, level of intercellular adhesion molecule 1 (ICAM1) in a sample from the individual, level of immediate early response 5 (IER5) in a sample from the individual, level of interferon α inducible protein 6 (IFI6) in a sample from the individual, level of interferon α inducible protein 27 (IFI27) in a sample from the individual, level of interferon induced protein 44 (IFI44) in a sample from the individual, level of interferon induced protein with tetratricopeptide repeats 1 (IFIT1) in a sample from the individual, level of interferon induced protein with tetratricopeptide repeats 2 (IFIT2) in a sample from the individual, level of interleukin 1β (IL1B) in a sample from the individual, level of interleukin 1 receptor type 1 (IL1 RA) in a sample from the individual, level of interleukin 1 receptor type 2 (IL1 R2) in a sample from the individual, level of interleukin 10 receptor subunit α (IL10RA) in a sample from the individual, level of interaction protein for cytohesin exchange factors 1 (IPCEF1) in a sample from the individual, level of interferon regulatory factor 2 binding protein 2 (IRF2BP2) in a sample from the individual, level of ISG15 ubiquitin like modifier (ISG15) in a sample from the individual, level of JUN proto-oncogene, AP-1 transcription factor subunit (JUN) in a sample from the individual, level of potassium voltage-gated channel subfamily E regulatory subunit 1 (KCNE1) in a sample from the individual, level of kinesin light chain 3 (KLC3) in a sample from the individual, level of kelch like family member 24 (KLHL24) in a sample from the individual, level of kringle containing transmembrane protein 1 (KREMEN1) in a sample from the individual, level of long intergenic non-protein coding RNA 861 (LINC00861) in a sample from the individual, level of lymphocyte antigen 6 family member E (LY6E) in a sample from the individual, level of MAPK associated protein 1 (MAPKAP1) in a sample from the individual, level of mediator complex subunit 28 (MED28) in a sample from the individual, level of MicroRNA 6724-4 (MIR6724-4) in a sample from the individual, level of matrix metallopeptidase 8 (MMP8) in a sample from the individual, level of multimerin 1 (MMRN1) in a sample from the individual, level of myeloperoxidase (MPO) in a sample from the individual, level of mannose receptor C type 2 (MRC2) in a sample from the individual, level of mitochondrially encoded 12S rRNA (MT-RNR1) in a sample from the individual, level of MX dynamin like GTPase 2 (MX2) in a sample from the individual, level of nuclear factor, erythroid 2 like 3 (NFE2L3) in a sample from the individual, level of 2′-5′-oligoadenylate synthetase 3 (OAS3) in a sample from the individual, level of oleoyl-ACP hydrolase (OLAH) in a sample from the individual, level of olfactomedin 4 (OLFM4) in a sample from the individual, level of peptidase inhibitor 3 (PI3) in a sample from the individual, level of phosphatidylinositol-4,5-bisphosphate 3-kinase catalytic subunit β (PIK3CB) in a sample from the individual, level of PITH domain containing 1 (PITHD1) in a sample from the individual, level of pyruvate kinase M1/2 (PKM) in a sample from the individual, level of perilipin 2 (PLIN2) in a sample from the individual, level of DNA polymerase δ interacting protein 3 (POLDIP3) in a sample from the individual, level of RAL GTPase activating protein catalytic subunit α2 (RALGAPA2) in a sample from the individual, level of RAN binding protein 9 (RANBP9) in a sample from the individual, level of REST corepressor 1 (RCOR1) in a sample from the individual, level of Rh associated glycoprotein (RHAG) in a sample from the individual, level of RNA, U1 small nuclear 2 (RNU1-2) in a sample from the individual, level of RNA, U1 small nuclear 4 (RNU1-4) in a sample from the individual, level of ribosomal protein L37a (RPL37A) in a sample from the individual, level of ribosomal protein L38 (RPL38) in a sample from the individual, level of ribosomal protein S11 (RPS11) in a sample from the individual, level of ribosomal protein S18 (RPS18) in a sample from the individual, level of radical S-adenosyl methionine domain containing 2 (RSAD2) in a sample from the individual, level of S100 calcium binding protein A8 (S100A8) in a sample from the individual, level of S100 calcium binding protein A9 (S100A9) in a sample from the individual, level of S100 calcium binding protein A12 (S100A12) in a sample from the individual, level of SAM domain, SH3 domain and nuclear localization signals 1 (SAMSN1) in a sample from the individual, level of Sin3A associated protein 30 (SAP30) in a sample from the individual, level of strawberry notch homolog 1 (SBNO1) in a sample from the individual, level of selenium binding protein 1 (SELENBP1) in a sample from the individual, level of sialic acid binding Ig like lectin 10 (SIGLEC10) in a sample from the individual, level of solute carrier family 25 member 6 (SLC25A6) in a sample from the individual, level of solute carrier family 25 member 39 (SLC25A39) in a sample from the individual, level of solute carrier family 39 member 8 (SLC39A8) in a sample from the individual, level of solute carrier family 4 member 1 [Diego Blood Group] (SLC4A1) in a sample from the individual, level of synuclein α (SNCA) in a sample from the individual, level of small nucleolar RNA, H/ACA box 44 (SNORA44) in a sample from the individual, level of superoxide dismutase 2 (SOD2) in a sample from the individual, level of spectrin α, erythrocytic 1 (SPTA1) in a sample from the individual, level of STE20 related adaptor β (STRADB) in a sample from the individual, level of syntaxin 6 (STX6) in a sample from the individual, level of switching B cell complex subunit SWAP70 (SWAP70) in a sample from the individual, level of spectrin repeat containing nuclear envelope protein 2 (SYNE2) in a sample from the individual, level of T-box transcription factor 21 (TBX21) in a sample from the individual, level of TRAF interacting protein with forkhead associated domain (TIFA) in a sample from the individual, level of toll like receptor 7 (TLR7) in a sample from the individual, level of transmembrane and coiled-coil domain family 2 (TMCC2) in a sample from the individual, level of transmembrane protein 35B (TMEM35B) in a sample from the individual, level of transmembrane protein 273 (TMEM273) in a sample from the individual, level of thymosin β10 (TMSB10) in a sample from the individual, level of TNF α induced protein 6 (TNFAIP6) in a sample from the individual, level of tyrosylprotein sulfotransferase 1 (TPST1) in a sample from the individual, level of tripartite motif containing 4 (TRIM4) in a sample from the individual, level of tetraspanin 5 (TSPAN5) in a sample from the individual, level of tetratricopeptide repeat domain 9C (TTC9C) in a sample from the individual, level of ubiquitin protein ligase E3 component N-recognin 5 (UBR5) in a sample from the individual, level of UNC-93 homolog B1, TLR signaling regulator (UNC93B1) in a sample from the individual, level of WASH complex subunit 2C (WASHC2C) in a sample from the individual, level of XIAP associated factor 1 (XAF1) in a sample from the individual, level of tyrosine 3-monooxygenase/tryptophan 5-monooxygenase activation protein E (YWHAH) in a sample from the individual, or level of or zinc finger With KRAB and SCAN domains 1 (ZKSCAN1) in a sample from the individual;
wherein the protein data markers comprise one or more of: level of a disintegrin and metalloproteinase with thrombospondin motifs 13 (ADAMTS13) in a sample from the individual, level of Angiopoietin-1 (ANGPT1) in a sample from the individual, level of Angiopoietin-2 (ANGPT2) in a sample from the individual, level of C-C chemokine receptor ligand 2/monocyte chemoattractant protein 1 (CCL2/MCP-1) in a sample from the individual, level of C-C chemokine receptor ligand 3/macrophage inflammatory protein 1-alpha (CCL3/MIP-1-α) in a sample from the individual, level of C-C chemokine receptor ligand 5/regulated on activation, normal T cell expressed and secreted (CCL5/RANTES) in a sample from the individual, level of cluster of differentiation 163 (CD163) in a sample from the individual, level of cluster of differentiation 40 ligand (CD40L) in a sample from the individual, level of chitinase-3-like protein 1 (CHI3L1) in a sample from the individual, level of C-reactive protein (CRP) in a sample from the individual, level of C-X-C motif chemokine ligand 10/interferon gamma-induced protein 10 (CXCL10/IP-10) in a sample from the individual, level of decoy receptor 3 (Dcr3) in a sample from the individual, level of D-dimer in a sample from the individual, level of E-selectin (SELE) in a sample from the individual, level of endoglin (ENG) in a sample from the individual, level of fas receptor (FAS) in a sample from the individual, level of ferritins in a sample from the individual, level of fibrinogens in a sample from the individual, level of granulocyte colony-stimulating factor (G-CSF) in a sample from the individual, level of granulocyte-macrophage colony-stimulating factor (GM-CSF) in a sample from the individual, level of (soluble) intercellular adhesion molecule 1 (ICAM-1) in a sample from the individual, level of interferon gamma (IFNγ) in a sample from the individual, level of interleukin 1 beta (IL-1β) in a sample from the individual, level of interleukin-1 receptor antagonist (IL-1RA) in a sample from the individual, level of (soluble) interleukin-2 receptor α (IL-2Rα) in a sample from the individual, level of interleukin-4 (IL-4) in a sample from the individual, level of interleukin-5 (IL-5) in a sample from the individual, level of interleukin-6 (IL-6) in a sample from the individual, level of interleukin-6 receptor α (IL-6Rα) in a sample from the individual, level of interleukin-7 (IL-7) in a sample from the individual, level of interleukin-8 (IL-8) in a sample from the individual, level of interleukin-10 (IL-10) in a sample from the individual, level of interleukin-12 ‘p70’ (IL-12 p70) in a sample from the individual, level of interleukin-15 (IL-15) in a sample from the individual, level of interleukin-16 (IL-16) in a sample from the individual, level of interleukin-17A (IL-17A) in a sample from the individual, level of interleukin-18 (IL-18) in a sample from the individual, level of interleukin-18-binding protein (IL-18BP) in a sample from the individual, level of interleukin-22 (IL-22) in a sample from the individual, level of interleukin-27 (IL-27) in a sample from the individual, level of lipocalin-2 (LCN-2) in a sample from the individual, level of matrix metalloproteinase-8 (MMP-8) in a sample from the individual, level of matrix metalloproteinase-9 (MMP-9) in a sample from the individual, level of matrix metalloproteinase-10 (MMP-10) in a sample from the individual, level of (soluble) macrophage mannose receptors in a sample from the individual, level of procalcitonin (PCT) in a sample from the individual, level of (soluble) programmed death-ligand 1 (PD-L1) in a sample from the individual, level of pentraxin 3 (PTX3) in a sample from the individual, level of (soluble) receptor for advanced glycation end products (RAGE) in a sample from the individual, level of resistin (RETN) in a sample from the individual, level of serum amyloid A proteins (SAA) in a sample from the individual, level of tyrosine kinase with immunoglobulin-like and EGF-like domains 1 (TIE1) in a sample from the individual, level of tyrosine kinase with immunoglobulin-like and EGF-like domains 2 (TIE2) in a sample from the individual, level of tissue inhibitor of metalloproteinases 1 (TIMP1) in a sample from the individual, level of tissue inhibitor of metalloproteinases 2 (TIMP2) in a sample from the individual, level of tissue inhibitor of metalloproteinases 3 (TIMP3) in a sample from the individual, level of tissue inhibitor of metalloproteinases 4 (TIMP4) in a sample from the individual, level of tumor necrosis factor receptor 1 (TNF-R1) in a sample from the individual, level of tumor necrosis factor alpha (TNFα) in a sample from the individual, level of tissue plasminogen activator (tPA) in a sample from the individual, level of tissue plasminogen activator inhibitor 1 (tPAI-1) in a sample from the individual, level of TNF-related apoptosis-inducing ligand (TRAIL) in a sample from the individual, level of (soluble) triggering receptor expressed on myeloid cells 1 (TREM1) in a sample from the individual, level of urokinase receptor (uPar) in a sample from the individual, level of (soluble) vascular cell adhesion molecule 1 (VCAM-1) in a sample from the individual, level of vascular endothelial growth factors (VEGF) in a sample from the individual, level of (soluble) vascular endothelial growth factor receptor 1 (VEGFR-1) in a sample from the individual, level of (soluble) vascular endothelial growth factor receptor 2 (VEGFR-2) in a sample from the individual, or level of von Willebrand factor A2 domain (vWF-A2) in a sample from the individual;
wherein the metabolite data comprise one or more of: levels of fatty acyls and their constituent molecular species in a sample from the individual, levels of glycerolipids and their constituent molecular species in a sample from the individual, levels of glycerophospholipids and their constituent molecular species in a sample from the individual, levels of sphingolipids and their constituent molecular species in a sample from the individual, levels of sterol lipids and their constituent molecular species in a sample from the individual, levels of prenol lipids and their constituent molecular species in a sample from the individual, levels of saccharolipids and their constituent molecular species in a sample from the individual, levels of polyketides and their constituent molecular species in a sample from the individual, levels of carbohydrates and their constituent molecular species in a sample from the individual, levels of organic acids and their derivatives and constituent molecular species in a sample from the individual, levels of organo-heterocyclic compounds and their constituent molecular species in a sample from the individual, levels of organo-oxygen compounds and their constituent molecular species in a sample from the individual, levels of organo-nitrogen compounds and their constituent molecular species in a sample from the individual, levels of amino acids and their constituent molecular species in a sample from the individual, levels of peptides and their constituent molecular species in a sample from the individual, or levels of nucleosides and their constituent molecular species in a sample from the individual;
wherein the clinical outcome data comprise one or more of: severity or duration of symptoms, time to symptom onset or abatement, need for organ support, duration of organ support, response to treatment, admission to a hospital or intensive care unit, length of stay in a hospital or intensive care unit, mortality, time to death, duration of morbidity (e.g., time to returning to normal daily activities or quality of life), incidence of long-term sequelae of infectious diseases, or re-hospitalization; and
wherein the administrative health data comprise one or more of: baseline demographics, physiologic parameters, comorbid conditions including but not limited to immunocompromising conditions, past surgical history, and environmental or social exposures.
10. A method for predicting severe disease in an individual with sepsis or at risk of developing sepsis comprising: receiving, from a second individual, a second value of at least one clinical parameter of a plurality of clinical parameters; executing a pre-trained model for predicting severe disease from sepsis of the second individual using the second value of at least one clinical parameter, wherein the model is pre-trained by performing operations comprising: generating a discovery database storing first values of the plurality of clinical parameters and clinical outcomes associated with a plurality of first subjects; executing a plurality of data quality control algorithms to select a subset of clinical parameters from the plurality of clinical parameters; executing topological data analysis and/or clustering for the plurality of subsets of clinical parameters; executing a plurality of feature selection machine learning and/or ensemble learning models based on a plurality of classification and/or time-to-event analysis algorithms; and outputting a model for predicting severe disease in the individual with sepsis or at risk of developing sepsis; and outputting the predicted mortality outcomes of the second individual.
11. The method of embodiment 10, further comprising pre-processing data that is stored in the discovery database including: determining that a first value of at least one of the plurality of clinical parameters is missing; estimating a reference value for the at least one of the plurality of clinical parameters that is missing; and storing the reference value as the first value of the at least one of the plurality of clinical parameters in the discovery database.
12. The method of embodiment 10 or 11, wherein the plurality of data quality control algorithms comprise at least one of: differential expression algorithms, k-nearest neighbor imputation algorithms, three-sigma rule algorithms, or empirical Bayes method algorithms.
13. The method of any one of embodiments 10-12, wherein the topological data analysis groups individuals or samples based on similarities in the plurality of subsets of clinical parameters, as well as the algebraic topology of the same data, wherein clusters are delineated based on the persistence homology of the node density and connectivity, and wherein sepsis response phenotypes are defined based on the identified clusters.
14. The method of any one of embodiments 10-13, wherein the cluster analysis discretizes the plurality of subsets of clinical parameters based on measures of similarity, wherein clusters are delineated based on the persistence homology of the node density and connectivity, and wherein sepsis response phenotypes are defined based on the identified clusters.
15. The method of any one of embodiments 10-14, wherein the feature selection machine learning models comprise at least one of: unsupervised machine learning algorithm, supervised machine learning algorithm, minimum redundancy maximum relevance, Student's t-test, Mann-Whitney U test, random forest, logistic regression, or neural networks.
16. The method of any one of embodiments 10-15, wherein the feature selection ensemble learning models comprise at least one of: cluster analysis, unsupervised machine learning algorithm, supervised machine learning algorithm, minimum redundancy maximum relevance, Student's t-test, Mann-Whitney U test, random forest, logistic regression, neural networks, Bayes optimal classifier, classification and regression tree, bootstrap aggregating, boosting, Bayesian model averaging, Bayesian model combination, a bucket of models, or stacking.
17. The method of any one of embodiments 10-17, wherein the plurality of clinical parameters comprise one or more nucleic acid data markers, one or more protein data markers, one or more of metabolite data markers, one or more clinical outcomes data, one or more administrative health data, or a combination thereof.
18. The method of embodiment 17, wherein the nucleic acid data markers comprise one or more of: level of adhesion G protein-coupled receptor E1 (ADGRE1) in a sample from the individual, level of adrenoceptor β2 (ADRB2) in a sample from the individual, level of angiotensin II receptor associated protein (AGTRAP) in a sample from the individual, level of AKT serine/threonine kinase 1 (AKT1) in a sample from the individual, level of 5′-aminolevulinate synthase 2 (ALAS2) in a sample from the individual, level of alkaline phosphatase, biomineralization associated (ALPL) in a sample from the individual, level of ankyrin repeat domain 22 (ANKRD22) in a sample from the individual, level of annexin A3 (ANXA3) in a sample from the individual, level of arginase 1 (ARG1) in a sample from the individual, level of BCL2 like 1 (BCL2L1) in a sample from the individual, level of BMX non-receptor tyrosine kinase (BMX) in a sample from the individual, level of chromosome 6 open reading frame 62 (C6orf62) in a sample from the individual, level of carbonic anhydrase 2 (CA2) in a sample from the individual, level of C-C motif chemokine ligand 5 (CCL5) in a sample from the individual, level of C-C motif chemokine receptor 3 (CCR3) in a sample from the individual, level of CD4 molecule (CD4) in a sample from the individual, level of CD24 molecule (CD24) in a sample from the individual, level of CD177 molecule (CD177) in a sample from the individual, level of CD274 molecule (CD274) in a sample from the individual, level of cell division cycle 34, ubiqiutin conjugating enzyme (CDC34) in a sample from the individual, level of complement factor D (CFD) in a sample from the individual, level of chitinase 3 like 1 (CHI3L1) in a sample from the individual, level of carbohydrate sulfotransferase 2 (CHST2) in a sample from the individual, level of C-type lectin domain family 4 member E (CLEC4E) in a sample from the individual, level of cytidine/uridine monophosphate kinase 2 (CMPK2) in a sample from the individual, level of cytochrome C oxidase assembly factor 1 homolog (COA1) in a sample from the individual, level of carnitine palmitoyltransferase 1A (CPT1A) in a sample from the individual, level of carboxypeptidase vitellogenic like (CPVL) in a sample from the individual, level of chondroitin sulfate N-acetylgalactosaminyltransferase 1 (CSGALNACT1) in a sample from the individual, level of cystatin C (CST3) in a sample from the individual, level of C-X3-C motif chemokine receptor 1 (CX3CR1) in a sample from the individual, level of DNA damage inducible transcript 4 (DDIT4) in a sample from the individual, level of defensin α3 (DEFA3) in a sample from the individual, level of defensin α4 (DEFA4) in a sample from the individual, level of DNA J heat shock protein family (Hsp40) member C1 (DNAJC1) in a sample from the individual, level of DNA damage regulated autophagy modulator 1 (DRAM1) in a sample from the individual, level of deoxyuridine triphosphatase (DUT) in a sample from the individual, level of dual specificity tyrosine phosphorylation regulated kinase 3 (DYRK3) in a sample from the individual, level of erythrocyte membrane protein band 4.2 (EPB42) in a sample from the individual, level of family with sequence similarity 174 member C (FAM174C) in a sample from the individual, level of F-box and WD repeat domain containing 2 (FBXW2) in a sample from the individual, level of Fc receptor like 5 (FCRL5) in a sample from the individual, level of ferrochelatase (FECH) in a sample from the individual, level of fibroblast growth factor binding protein 2 (FGFBP2) in a sample from the individual, level of Fms related receptor tyrosine kinase 3 (FLT3) in a sample from the individual, level of formyl peptide receptor 1 (FPR1) in a sample from the individual, level of GATA binding protein 1 (GATA1) in a sample from the individual, level of GTPase, IMAP family member 4 (GIMAP4) in a sample from the individual, level of GTPase, IMAP family member 7 (GIMAP7) in a sample from the individual, level of GTPase, IMAP family member 8 (GIMAP8) in a sample from the individual, level of G protein subunit γ2 (GNG2) in a sample from the individual, level of granulysin (GNLY) in a sample from the individual, level of G protein-coupled receptor 65 (GPR65) in a sample from the individual, level of growth factor receptor bound protein 10 (GRB10) in a sample from the individual, level of glutathione S-transferase κ1 (GSTK1) in a sample from the individual, level of H3 histone pseudogene 6 (H3F3AP4) in a sample from the individual, level of hemoglobin subunit α2 (HBA2) in a sample from the individual, level of hemogen (HEMGN) in a sample from the individual, level of HECT and RLD domain containing E3 ubiquitin protein ligase family member 6 (HERC6) in a sample from the individual, level of H3.2 histone [putative] (HIST2H3PS2) in a sample from the individual, level of major histocompatibility complex, class I, B (HLA-B) in a sample from the individual, level of major histocompatibility complex, class II, DQ β1 (HLA-DQB1) in a sample from the individual, level of high mobility group box 2 (HMGB2) in a sample from the individual, level of 15-hydroxyprostaglandin dehydrogenase (HPGD) in a sample from the individual, level of hydrogen voltage gated channel 1 (HVCN1) in a sample from the individual, level of isoamyl acetate hydrolyzing esterase 1 [putative] (IAH1) in a sample from the individual, level of intercellular adhesion molecule 1 (ICAM1) in a sample from the individual, level of immediate early response 5 (IER5) in a sample from the individual, level of interferon α inducible protein 6 (IFI6) in a sample from the individual, level of interferon α inducible protein 27 (IFI27) in a sample from the individual, level of interferon induced protein 44 (IFI44) in a sample from the individual, level of interferon induced protein with tetratricopeptide repeats 1 (IFIT1) in a sample from the individual, level of interferon induced protein with tetratricopeptide repeats 2 (IFIT2) in a sample from the individual, level of interleukin 1β (IL1B) in a sample from the individual, level of interleukin 1 receptor type 1 (IL1RA) in a sample from the individual, level of interleukin 1 receptor type 2 (IL1R2) in a sample from the individual, level of interleukin 10 receptor subunit α (IL10RA) in a sample from the individual, level of interaction protein for cytohesin exchange factors 1 (IPCEF1) in a sample from the individual, level of interferon regulatory factor 2 binding protein 2 (IRF2BP2) in a sample from the individual, level of ISG15 ubiquitin like modifier (ISG15) in a sample from the individual, level of JUN proto-oncogene, AP-1 transcription factor subunit (JUN) in a sample from the individual, level of potassium voltage-gated channel subfamily E regulatory subunit 1 (KCNE1) in a sample from the individual, level of kinesin light chain 3 (KLC3) in a sample from the individual, level of kelch like family member 24 (KLHL24) in a sample from the individual, level of kringle containing transmembrane protein 1 (KREMEN1) in a sample from the individual, level of long intergenic non-protein coding RNA 861 (LINC00861) in a sample from the individual, level of lymphocyte antigen 6 family member E (LY6E) in a sample from the individual, level of MAPK associated protein 1 (MAPKAP1) in a sample from the individual, level of mediator complex subunit 28 (MED28) in a sample from the individual, level of MicroRNA 6724-4 (MIR6724-4) in a sample from the individual, level of matrix metallopeptidase 8 (MMP8) in a sample from the individual, level of multimerin 1 (MMRN1) in a sample from the individual, level of myeloperoxidase (MPO) in a sample from the individual, level of mannose receptor C type 2 (MRC2) in a sample from the individual, level of mitochondrially encoded 12S rRNA (MT-RNR1) in a sample from the individual, level of MX dynamin like GTPase 2 (MX2) in a sample from the individual, level of nuclear factor, erythroid 2 like 3 (NFE2L3) in a sample from the individual, level of 2′-5′-oligoadenylate synthetase 3 (OAS3) in a sample from the individual, level of oleoyl-ACP hydrolase (OLAH) in a sample from the individual, level of olfactomedin 4 (OLFM4) in a sample from the individual, level of peptidase inhibitor 3 (PI3) in a sample from the individual, level of phosphatidylinositol-4,5-bisphosphate 3-kinase catalytic subunit β (PIK3CB) in a sample from the individual, level of PITH domain containing 1 (PITHD1) in a sample from the individual, level of pyruvate kinase M1/2 (PKM) in a sample from the individual, level of perilipin 2 (PLIN2) in a sample from the individual, level of DNA polymerase δ interacting protein 3 (POLDIP3) in a sample from the individual, level of RAL GTPase activating protein catalytic subunit α2 (RALGAPA2) in a sample from the individual, level of RAN binding protein 9 (RANBP9) in a sample from the individual, level of REST corepressor 1 (RCOR1) in a sample from the individual, level of Rh associated glycoprotein (RHAG) in a sample from the individual, level of RNA, U1 small nuclear 2 (RNU1-2) in a sample from the individual, level of RNA, U1 small nuclear 4 (RNU1-4) in a sample from the individual, level of ribosomal protein L37a (RPL37A) in a sample from the individual, level of ribosomal protein L38 (RPL38) in a sample from the individual, level of ribosomal protein S11 (RPS11) in a sample from the individual, level of ribosomal protein S18 (RPS18) in a sample from the individual, level of radical S-adenosyl methionine domain containing 2 (RSAD2) in a sample from the individual, level of S100 calcium binding protein A8 (S100A8) in a sample from the individual, level of S100 calcium binding protein A9 (S100A9) in a sample from the individual, level of S100 calcium binding protein A12 (S100A12) in a sample from the individual, level of SAM domain, SH3 domain and nuclear localization signals 1 (SAMSN1) in a sample from the individual, level of Sin3A associated protein 30 (SAP30) in a sample from the individual, level of strawberry notch homolog 1 (SBNO1) in a sample from the individual, level of selenium binding protein 1 (SELENBP1) in a sample from the individual, level of sialic acid binding Ig like lectin 10 (SIGLEC10) in a sample from the individual, level of solute carrier family 25 member 6 (SLC25A6) in a sample from the individual, level of solute carrier family 25 member 39 (SLC25A39) in a sample from the individual, level of solute carrier family 39 member 8 (SLC39A8) in a sample from the individual, level of solute carrier family 4 member 1 [Diego Blood Group] (SLC4A1) in a sample from the individual, level of synuclein α (SNCA) in a sample from the individual, level of small nucleolar RNA, H/ACA box 44 (SNORA44) in a sample from the individual, level of superoxide dismutase 2 (SOD2) in a sample from the individual, level of spectrin α, erythrocytic 1 (SPTA1) in a sample from the individual, level of STE20 related adaptor β (STRADB) in a sample from the individual, level of syntaxin 6 (STX6) in a sample from the individual, level of switching B cell complex subunit SWAP70 (SWAP70) in a sample from the individual, level of spectrin repeat containing nuclear envelope protein 2 (SYNE2) in a sample from the individual, level of T-box transcription factor 21 (TBX21) in a sample from the individual, level of TRAF interacting protein with forkhead associated domain (TIFA) in a sample from the individual, level of toll like receptor 7 (TLR7) in a sample from the individual, level of transmembrane and coiled-coil domain family 2 (TMCC2) in a sample from the individual, level of transmembrane protein 35B (TMEM35B) in a sample from the individual, level of transmembrane protein 273 (TMEM273) in a sample from the individual, level of thymosin β10 (TMSB10) in a sample from the individual, level of TNF α induced protein 6 (TNFAIP6) in a sample from the individual, level of tyrosylprotein sulfotransferase 1 (TPST1) in a sample from the individual, level of tripartite motif containing 4 (TRIM4) in a sample from the individual, level of tetraspanin 5 (TSPAN5) in a sample from the individual, level of tetratricopeptide repeat domain 9C (TTC9C) in a sample from the individual, level of ubiquitin protein ligase E3 component N-recognin 5 (UBR5) in a sample from the individual, level of UNC-93 homolog B1, TLR signaling regulator (UNC93B1) in a sample from the individual, level of WASH complex subunit 2C (WASHC2C) in a sample from the individual, level of XIAP associated factor 1 (XAF1) in a sample from the individual, level of tyrosine 3-monooxygenase/tryptophan 5-monooxygenase activation protein E (YWHAH) in a sample from the individual, or level of or zinc finger With KRAB and SCAN domains 1 (ZKSCAN1) in a sample from the individual;
wherein the protein data markers comprise one or more of: level of a disintegrin and metalloproteinase with thrombospondin motifs 13 (ADAMTS13) in a sample from the individual, level of Angiopoietin-1 (ANGPT1) in a sample from the individual, level of Angiopoietin-2 (ANGPT2) in a sample from the individual, level of C-C chemokine receptor ligand 2/monocyte chemoattractant protein 1 (CCL2/MCP-1) in a sample from the individual, level of C-C chemokine receptor ligand 3/macrophage inflammatory protein 1-alpha (CCL3/MIP-1-α) in a sample from the individual, level of C-C chemokine receptor ligand 5/regulated on activation, normal T cell expressed and secreted (CCL5/RANTES) in a sample from the individual, level of cluster of differentiation 163 (CD163) in a sample from the individual, level of cluster of differentiation 40 ligand (CD40L) in a sample from the individual, level of chitinase-3-like protein 1 (CHI3L1) in a sample from the individual, level of C-reactive protein (CRP) in a sample from the individual, level of C-X-C motif chemokine ligand 10/interferon gamma-induced protein 10 (CXCL10/IP-10) in a sample from the individual, level of decoy receptor 3 (Dcr3) in a sample from the individual, level of D-dimer in a sample from the individual, level of E-selectin (SELE) in a sample from the individual, level of endoglin (ENG) in a sample from the individual, level of fas receptor (FAS) in a sample from the individual, level of ferritins in a sample from the individual, level of fibrinogens in a sample from the individual, level of granulocyte colony-stimulating factor (G-CSF) in a sample from the individual, level of granulocyte-macrophage colony-stimulating factor (GM-CSF) in a sample from the individual, level of (soluble) intercellular adhesion molecule 1 (ICAM-1) in a sample from the individual, level of interferon gamma (IFNγ) in a sample from the individual, level of interleukin 1 beta (IL-1β) in a sample from the individual, level of interleukin-1 receptor antagonist (IL-1RA) in a sample from the individual, level of (soluble) interleukin-2 receptor α (IL-2Rα) in a sample from the individual, level of interleukin-4 (IL-4) in a sample from the individual, level of interleukin-5 (IL-5) in a sample from the individual, level of interleukin-6 (IL-6) in a sample from the individual, level of interleukin-6 receptor α (IL-6Rα) in a sample from the individual, level of interleukin-7 (IL-7) in a sample from the individual, level of interleukin-8 (IL-8) in a sample from the individual, level of interleukin-10 (IL-10) in a sample from the individual, level of interleukin-12 ‘p70’ (IL-12 p70) in a sample from the individual, level of interleukin-15 (IL-15) in a sample from the individual, level of interleukin-16 (IL-16) in a sample from the individual, level of interleukin-17A (IL-17A) in a sample from the individual, level of interleukin-18 (IL-18) in a sample from the individual, level of interleukin-18-binding protein (IL-18BP) in a sample from the individual, level of interleukin-22 (IL-22) in a sample from the individual, level of interleukin-27 (IL-27) in a sample from the individual, level of lipocalin-2 (LCN-2) in a sample from the individual, level of matrix metalloproteinase-8 (MMP-8) in a sample from the individual, level of matrix metalloproteinase-9 (MMP-9) in a sample from the individual, level of matrix metalloproteinase-10 (MMP-10) in a sample from the individual, level of (soluble) macrophage mannose receptors in a sample from the individual, level of procalcitonin (PCT) in a sample from the individual, level of (soluble) programmed death-ligand 1 (PD-L1) in a sample from the individual, level of pentraxin 3 (PTX3) in a sample from the individual, level of (soluble) receptor for advanced glycation end products (RAGE) in a sample from the individual, level of resistin (RETN) in a sample from the individual, level of serum amyloid A proteins (SAA) in a sample from the individual, level of tyrosine kinase with immunoglobulin-like and EGF-like domains 1 (TIE1) in a sample from the individual, level of tyrosine kinase with immunoglobulin-like and EGF-like domains 2 (TIE2) in a sample from the individual, level of tissue inhibitor of metalloproteinases 1 (TIMP1) in a sample from the individual, level of tissue inhibitor of metalloproteinases 2 (TIMP2) in a sample from the individual, level of tissue inhibitor of metalloproteinases 3 (TIMP3) in a sample from the individual, level of tissue inhibitor of metalloproteinases 4 (TIMP4) in a sample from the individual, level of tumor necrosis factor receptor 1 (TNF-R1) in a sample from the individual, level of tumor necrosis factor alpha (TNFα) in a sample from the individual, level of tissue plasminogen activator (tPA) in a sample from the individual, level of tissue plasminogen activator inhibitor 1 (tPAI-1) in a sample from the individual, level of TNF-related apoptosis-inducing ligand (TRAIL) in a sample from the individual, level of (soluble) triggering receptor expressed on myeloid cells 1 (TREM1) in a sample from the individual, level of urokinase receptor (uPar) in a sample from the individual, level of (soluble) vascular cell adhesion molecule 1 (VCAM-1) in a sample from the individual, level of vascular endothelial growth factors (VEGF) in a sample from the individual, level of (soluble) vascular endothelial growth factor receptor 1 (VEGFR-1) in a sample from the individual, level of (soluble) vascular endothelial growth factor receptor 2 (VEGFR-2) in a sample from the individual, or level of von Willebrand factor A2 domain (vWF-A2) in a sample from the individual;
wherein the metabolite data comprise one or more of: levels of fatty acyls and their constituent molecular species in a sample from the individual, levels of glycerolipids and their constituent molecular species in a sample from the individual, levels of glycerophospholipids and their constituent molecular species in a sample from the individual, levels of sphingolipids and their constituent molecular species in a sample from the individual, levels of sterol lipids and their constituent molecular species in a sample from the individual, levels of prenol lipids and their constituent molecular species in a sample from the individual, levels of saccharolipids and their constituent molecular species in a sample from the individual, levels of polyketides and their constituent molecular species in a sample from the individual, levels of carbohydrates and their constituent molecular species in a sample from the individual, levels of organic acids and their derivatives and constituent molecular species in a sample from the individual, levels of organo-heterocyclic compounds and their constituent molecular species in a sample from the individual, levels of organo-oxygen compounds and their constituent molecular species in a sample from the individual, levels of organo-nitrogen compounds and their constituent molecular species in a sample from the individual, levels of amino acids and their constituent molecular species in a sample from the individual, levels of peptides and their constituent molecular species in a sample from the individual, or levels of nucleosides and their constituent molecular species in a sample from the individual;
wherein the clinical outcome data comprise one or more of: severity or duration of symptoms, time to symptom onset or abatement, need for organ support, duration of organ support, response to treatment, admission to a hospital or intensive care unit, length of stay in a hospital or intensive care unit, mortality, time to death, duration of morbidity (e.g., time to returning to normal daily activities or quality of life), incidence of long-term sequelae of infectious diseases, or re-hospitalization; and
wherein the administrative health data comprise one or more of: baseline demographics, physiologic parameters, comorbid conditions including but not limited to immunocompromising conditions, past surgical history, and environmental or social exposures.
19. The method of any one of embodiments 10-18, wherein the method further comprises treating the individual or adjusting current treatment for the individual to prevent or ameliorate severe disease from sepsis based on the model.
20. The method of any one of embodiments 10-19, wherein treating the individual comprises at least one of initiation or broadening of antibiotic therapy, balancing fluids and electrolytes, renal replacement therapy, adjustment of mechanical ventilation, targeted or empiric anti-inflammatory or immunomodulatory drugs, hemodynamic adjustments, calcium channel blocker medications, or surgical intervention.
21. The method of any one of embodiments 10-19, wherein adjusting current treatment comprises changing dose of current antibiotic, changing to a different antibiotic, changing dose of non-steroidal anti-inflammatory drugs, initiating or adjusting insulin therapy.
22. A system for generating a machine learning engine for predicting severe disease in an individual with sepsis or at risk of developing sepsis, comprising: one or more processors; a memory; a communication platform; a discovery database configured to store first values of a plurality of clinical parameters and clinical outcomes associated with a plurality of first subjects; and a machine learning engine configured to: execute a plurality of data quality control algorithms to select a subset of clinical parameters from the plurality of clinical parameters; execute topological data analysis and/or clustering for the plurality of subsets of clinical parameters; execute a plurality of feature selection machine learning and/or ensemble learning models based on a plurality of classification and/or time-to-event analysis algorithms; and output a model for predicting severe disease in the individual with sepsis or at risk of developing sepsis.
23. The system of embodiment 22, further comprising configuring the model to predict severe disease in the individual with sepsis or at risk of developing sepsis and instantiating the model in a prediction engine that is accessed by a remote device connected to the system via a network.
24. The system of embodiment 22 or 23, wherein the communication platform comprises at least one of: a mobile device, a secured network, a server that stores and receives messages, and a database.
25. A system for predicting severe disease in an individual with sepsis or at risk of developing sepsis, comprising: one or more processors; a memory; a communication platform; a discovery database configured to store first values of a plurality of clinical parameters and the clinical outcomes associated with a plurality of first subjects; a machine learning engine configured to pre-train a model for severe disease in an individual with sepsis or at risk of developing sepsis, wherein the model is pre-trained by performing operations comprising: executing a plurality of data quality control algorithms to select a subset of clinical parameters from the plurality of clinical parameters; executing topological data analysis and/or clustering for the plurality of subsets of clinical parameters; executing a plurality of feature selection machine learning and/or ensemble learning models based on a plurality of classification and/or time-to-event analysis algorithms; outputting a model for predicting severe disease in the individual with sepsis or at risk of developing sepsis; a prediction engine configured to: receive, from a second individual, a second value of at least one clinical parameter of a plurality of clinical parameters; and execute the pre-trained model for predicting severe disease of the second individual using the second value of at least one clinical parameter; and a display device configured to output the predicted severe disease of the second individual.
26. A non-transitory computer-readable medium having information recorded thereon for generating a model for predicting severe disease in an individual with sepsis or at risk of developing sepsis, wherein the information, when read by a computer, causes the computer to perform operations of: generating a discovery database storing first values of a plurality of clinical parameters and clinical outcomes associated with a plurality of first subjects; executing a plurality of data quality control algorithms to select a subset of clinical parameters from the plurality of clinical parameters; executing topological data analysis and/or clustering for the plurality of subsets of clinical parameters; executing a plurality of feature selection machine learning and/or ensemble learning models based on a plurality of classification and/or time-to-event analysis algorithms; and outputting a model for predicting severe disease in the individual with sepsis or at risk of developing sepsis.
27. An array of host-biomarkers for sepsis, wherein the array of biomarkers comprise two or more of: adhesion G protein-coupled receptor E1 (ADGRE1), adrenoceptor β2 (ADRB2), angiotensin II receptor associated protein (AGTRAP), AKT serine/threonine kinase 1 (AKT1), 5′-aminolevulinate synthase 2 (ALAS2), alkaline phosphatase, biomineralization associated (ALPL), ankyrin repeat domain 22 (ANKRD22), annexin A3 (ANXA3), arginase 1 (ARG1), BCL2 like 1 (BCL2L1), BMX non-receptor tyrosine kinase (BMX), chromosome 6 open reading frame 62 (C6orf62), carbonic anhydrase 2 (CA2), C-C motif chemokine ligand 5 (CCL5), C-C motif chemokine receptor 3 (CCR3), CD4 molecule (CD4), CD24 molecule (CD24), CD177 molecule (CD177), CD274 molecule (CD274), cell division cycle 34, ubiqiutin conjugating enzyme (CDC34), complement factor D (CFD), chitinase 3 like 1 (CHI3L1), carbohydrate sulfotransferase 2 (CHST2), C-type lectin domain family 4 member E (CLEC4E), cytidine/uridine monophosphate kinase 2 (CMPK2), cytochrome C oxidase assembly factor 1 homolog (COA1), carnitine palmitoyltransferase 1A (CPT1A), carboxypeptidase vitellogenic like (CPVL), chondroitin sulfate N-acetylgalactosaminyltransferase 1 (CSGALNACT1), cystatin C (CST3), C-X3-C motif chemokine receptor 1 (CX3CR1), DNA damage inducible transcript 4 (DDIT4), defensin α3 (DEFA3), defensin α4 (DEFA4), DNA J heat shock protein family (Hsp40) member C1 (DNAJC1), DNA damage regulated autophagy modulator 1 (DRAM1), deoxyuridine triphosphatase (DUT), dual specificity tyrosine phosphorylation regulated kinase 3 (DYRK3), erythrocyte membrane protein band 4.2 (EPB42), family with sequence similarity 174 member C (FAM174C), F-box and WD repeat domain containing 2 (FBXW2), Fc receptor like 5 (FCRL5), ferrochelatase (FECH), fibroblast growth factor binding protein 2 (FGFBP2), Fms related receptor tyrosine kinase 3 (FLT3), formyl peptide receptor 1 (FPR1), GATA binding protein 1 (GATA1), GTPase, IMAP family member 4 (GIMAP4), GTPase, IMAP family member 7 (GIMAP7), GTPase, IMAP family member 8 (GIMAP8), G protein subunit γ2 (GNG2), granulysin (GNLY), G protein-coupled receptor 65 (GPR65), growth factor receptor bound protein 10 (GRB10), glutathione S-transferase κ1 (GSTK1), H3 histone pseudogene 6 (H3F3AP4), hemoglobin subunit α2 (HBA2), hemogen (HEMGN), HECT and RLD domain containing E3 ubiquitin protein ligase family member 6 (HERC6), H3.2 histone [putative] (HIST2H3PS2), major histocompatibility complex, class I, B (HLA-B), major histocompatibility complex, class II, DQ β1 (HLA-DQB1), high mobility group box 2 (HMGB2), 15-hydroxyprostaglandin dehydrogenase (HPGD), hydrogen voltage gated channel 1 (HVCN1), isoamyl acetate hydrolyzing esterase 1 [putative] (IAH1), intercellular adhesion molecule 1 (ICAM1), immediate early response 5 (IER5), interferon α inducible protein 6 (IFI6), interferon α inducible protein 27 (IFI27), interferon induced protein 44 (IFI44), interferon induced protein with tetratricopeptide repeats 1 (IFIT1), interferon induced protein with tetratricopeptide repeats 2 (IFIT2), interleukin 1β (IL1B), interleukin 1 receptor type 1 (IL1RA), interleukin 1 receptor type 2 (IL1R2), interleukin 10 receptor subunit α (IL10RA), interaction protein for cytohesin exchange factors 1 (IPCEF1), interferon regulatory factor 2 binding protein 2 (IRF2BP2), ISG15 ubiquitin like modifier (ISG15), JUN proto-oncogene, AP-1 transcription factor subunit (JUN), potassium voltage-gated channel subfamily E regulatory subunit 1 (KCNE1), kinesin light chain 3 (KLC3), kelch like family member 24 (KLHL24), kringle containing transmembrane protein 1 (KREMEN1), long intergenic non-protein coding RNA 861 (LINC00861), lymphocyte antigen 6 family member E (LY6E), MAPK associated protein 1 (MAPKAP1), mediator complex subunit 28 (MED28), MicroRNA 6724-4 (MIR6724-4), matrix metallopeptidase 8 (MMP8), multimerin 1 (MMRN1), myeloperoxidase (MPO), mannose receptor C type 2 (MRC2), mitochondrially encoded 12S rRNA (MT-RNR1), MX dynamin like GTPase 2 (MX2), nuclear factor, erythroid 2 like 3 (NFE2L3), 2′-5′-oligoadenylate synthetase 3 (OAS3), oleoyl-ACP hydrolase (OLAH), olfactomedin 4 (OLFM4), peptidase inhibitor 3 (PI3), phosphatidylinositol-4,5-bisphosphate 3-kinase catalytic subunit β (PIK3CB), PITH domain containing 1 (PITHD1), pyruvate kinase M1/2 (PKM), perilipin 2 (PLIN2), DNA polymerase δ interacting protein 3 (POLDIP3), RAL GTPase activating protein catalytic subunit α2 (RALGAPA2), RAN binding protein 9 (RANBP9), REST corepressor 1 (RCOR1), Rh associated glycoprotein (RHAG), RNA, U1 small nuclear 2 (RNU1-2), RNA, U1 small nuclear 4 (RNU1-4), ribosomal protein L37a (RPL37A), ribosomal protein L38 (RPL38), ribosomal protein S11 (RPS11), ribosomal protein S18 (RPS18), radical S-adenosyl methionine domain containing 2 (RSAD2), S100 calcium binding protein A8 (S100A8), S100 calcium binding protein A9 (S100A9), S100 calcium binding protein A12 (S100A12), SAM domain, SH3 domain and nuclear localization signals 1 (SAMSN1), Sin3A associated protein 30 (SAP30), strawberry notch homolog 1 (SBNO1), selenium binding protein 1 (SELENBP1), sialic acid binding Ig like lectin 10 (SIGLEC10), solute carrier family 25 member 6 (SLC25A6), solute carrier family 25 member 39 (SLC25A39), solute carrier family 39 member 8 (SLC39A8), solute carrier family 4 member 1 [Diego Blood Group] (SLC4A1), synuclein α (SNCA), small nucleolar RNA, H/ACA box 44 (SNORA44), superoxide dismutase 2 (SOD2), spectrin α, erythrocytic 1 (SPTA1), STE20 related adaptor β (STRADB), syntaxin 6 (STX6), switching B cell complex subunit SWAP70 (SWAP70), spectrin repeat containing nuclear envelope protein 2 (SYNE2), T-box transcription factor 21 (TBX21), TRAF interacting protein with forkhead associated domain (TIFA), toll like receptor 7 (TLR7), transmembrane and coiled-coil domain family 2 (TMCC2), transmembrane protein 35B (TMEM35B), transmembrane protein 273 (TMEM273), thymosin β10 (TMSB10), TNF α induced protein 6 (TNFAIP6), tyrosylprotein sulfotransferase 1 (TPST1), tripartite motif containing 4 (TRIM4), tetraspanin 5 (TSPAN5), tetratricopeptide repeat domain 9C (TTC9C), ubiquitin protein ligase E3 component N-recognin 5 (UBR5), UNC-93 homolog B1, TLR signaling regulator (UNC93B1), WASH complex subunit 2C (WASHC2C), XIAP associated factor 1 (XAF1), tyrosine 3-monooxygenase/tryptophan 5-monooxygenase activation protein E (YWHAH), and zinc finger With KRAB and SCAN domains 1 (ZKSCAN1), a disintegrin and metalloproteinase with thrombospondin motifs 13 (ADAMTS13), Angiopoietin-1 (ANGPT1), Angiopoietin-2 (ANGPT2), C-C chemokine receptor ligand 2/monocyte chemoattractant protein 1 (CCL2/MCP-1), C-C chemokine receptor ligand 3/macrophage inflammatory protein 1-alpha (CCL3/MIP-1-α), C-C chemokine receptor ligand 5/regulated on activation, normal T cell expressed and secreted (CCL5/RANTES), cluster of differentiation 163 (CD163), cluster of differentiation 40 ligand (CD40L), chitinase-3-like protein 1 (CHI3L1), C-reactive protein (CRP), C-X-C motif chemokine ligand 10/interferon gamma-induced protein 10 (CXCL10/IP-10), decoy receptor 3 (Dcr3), D-dimer, E-selectin (SELE), endoglin (ENG), fas receptor (FAS), ferritins, fibrinogens, granulocyte colony-stimulating factor (G-CSF), granulocyte-macrophage colony-stimulating factor (GM-CSF), (soluble) intercellular adhesion molecule 1 (ICAM-1), interferon gamma (IFNγ), interleukin 1 beta (IL-1β), interleukin-1 receptor antagonist (IL-1RA), (soluble) interleukin-2 receptor α (IL-2Rα), interleukin-4 (IL-4), interleukin-5 (IL-5), interleukin-6 (IL-6), interleukin-6 receptor α (IL-6Rα), interleukin-7 (IL-7), interleukin-8 (IL-8), interleukin-10 (IL-10), interleukin-12 ‘p70’ (IL-12 p70), interleukin-15 (IL-15), interleukin-16 (IL-16), interleukin-17A (IL-17A), interleukin-18 (IL-18), interleukin-18-binding protein (IL-18BP), interleukin-22 (IL-22), interleukin-27 (IL-27), lipocalin-2 (LCN-2), matrix metalloproteinase-8 (MMP-8), matrix metalloproteinase-9 (MMP-9), matrix metalloproteinase-10 (MMP-10), (soluble) macrophage mannose receptors, procalcitonin (PCT), (soluble) programmed death-ligand 1 (PD-L1), pentraxin 3 (PTX3), (soluble) receptor for advanced glycation end products (RAGE), resistin (RETN), serum amyloid A proteins (SAA), tyrosine kinase with immunoglobulin-like and EGF-like domains 1 (TIE1), tyrosine kinase with immunoglobulin-like and EGF-like domains 2 (TIE2), tissue inhibitor of metalloproteinases 1 (TIMP1), tissue inhibitor of metalloproteinases 2 (TIMP2), tissue inhibitor of metalloproteinases 3 (TIMP3), tissue inhibitor of metalloproteinases 4 (TIMP4), tumor necrosis factor receptor 1 (TNF-R1), tumor necrosis factor alpha (TNFα), tissue plasminogen activator (tPA), tissue plasminogen activator inhibitor 1 (tPAI-1), TNF-related apoptosis-inducing ligand (TRAIL), (soluble) triggering receptor expressed on myeloid cells 1 (TREM1), urokinase receptor (uPar), (soluble) vascular cell adhesion molecule 1 (VCAM-1), vascular endothelial growth factors (VEGF), (soluble) vascular endothelial growth factor receptor 1 (VEGFR-1), (soluble) vascular endothelial growth factor receptor 2 (VEGFR-2), von Willebrand factor A2 domain (vWF-A2), fatty acyls and their constituent molecular species, glycerolipids and their constituent molecular species, glycerophospholipids and their constituent molecular species, sphingolipids and their constituent molecular species, sterol lipids and their constituent molecular species, prenol lipids and their constituent molecular species, saccharolipids and their constituent molecular species, polyketides and their constituent molecular species, carbohydrates and their constituent molecular species, organic acids and their derivatives and constituent molecular species, organo-heterocyclic compounds and their constituent molecular species, organo-oxygen compounds and their constituent molecular species, organo-nitrogen compounds and their constituent molecular species, amino acids and their constituent molecular species, peptides and their constituent molecular species, or nucleosides and their constituent molecular species.
28. The array of biomarkers of embodiment 27, wherein the array is an array of nucleic acids, an array of peptides, or an array of metabolites.
29. The array of biomarkers of embodiment 27 or 28, wherein the array comprises three or more biomarkers, four or more biomarkers, five or more biomarkers, six or more biomarkers, seven or more biomarkers, eight of more biomarkers, nine or more biomarkers, 10 or more biomarkers, 15 or more biomarkers, 20 or more biomarkers, 25 or more biomarkers, 30 or more biomarkers, 35 or more biomarkers, 40 or more biomarkers, 45 or more biomarkers, or 48 biomarkers
30. An array of biomarkers, wherein the array comprises two or more of the following biomarkers: adrenoceptor β2 (ADRB2), CD177 molecule (CD177), carboxypeptidase vitellogenic like (CPVL), C-X3-C motif chemokine receptor 1 (CX3CR1), defensin α3 (DEFA3), Fc receptor like 5 (FCRL5), G protein subunit γ2 (GNG2), interleukin 10 receptor subunit α (I10RA), kinesin light chain 3 (KLC3), oleoyl-ACP hydrolase (OLAH), pyruvate kinase M1/2 (PKM), radical S-adenosyl methionine domain containing 2 (RSAD2), STE20 related adaptor β (STRADB), tyrosylprotein sulfotransferase 1 (TPST1), tetraspanin 5 (TSPAN5), tetratricopeptide repeat domain 9C (TTC9C), zinc finger with KRAB and SCAN domains 1 (ZKSCAN1), C-reactive protein (CRP), C-X-C motif chemokine ligand 10/interferon gamma-induced protein 10 (CXCL10/IP-10), D-dimer, ferritins, fibrinogens, (soluble) intercellular adhesion molecule 1 (ICAM-1), interferon gamma (IFNγ), interleukin-1 receptor antagonist (IL-1RA), interleukin-5 (IL-5), interleukin-6 (IL-6), interleukin-6 receptor α (IL-6Rα), interleukin-18 (IL-18), interleukin-18-binding protein (IL-18BP), lipocalin-2 (LCN-2), matrix metalloproteinase-8 (MMP-8), procalcitonin (PCT), (soluble) receptor for advanced glycation end products (RAGE), tumor necrosis factor receptor 1 (TNF-R1), tumor necrosis factor alpha (TNFα), vascular endothelial growth factors (VEGF), von Willebrand factor A2 domain (vWF-A2), carnitine, acetylcarnitine, propionylcarnitine, malonylcarnitine, methylmalonylcarnitine, hydroxypropionylcarnitine, propenoylcarnitine, butyrylcarnitine, hydroxybutyrylcarnitine, fumarylcarnitine, valerylcarnitine, glutarylcarnitine, hydroxyvalerylcarnitine, tiglylcarnitine, hexanoylcarnitine, hydroxyhexanoylcarnitine, pimeloylcarnitine, decanoylcarnitine, decadienylcarnitine, tetradecenoylcarnitine, hydroxytetradecenoylcarnitine, hydroxytetradecadienylcarnitine, hexadecanoylcarnitine, hexadecenoylcarnitine, octadecanoylcarnitine, octadecenoylcarnitine, lysophosphatidylcholine with acyl residue sum C16:0, lysophosphatidylcholine with acyl residue sum C16:1, lysophosphatidylcholine with acyl residue sum C17:0, lysophosphatidylcholine with acyl residue sum C18:0, lysophosphatidylcholine with acyl residue sum C18:1, lysophosphatidylcholine with acyl residue sum C18:2, lysophosphatidylcholine with acyl residue sum C20:3, lysophosphatidylcholine with acyl residue sum C20:4, lysophosphatidylcholine with acyl residue sum C24:0, lysophosphatidylcholine with acyl residue sum C26:0, lysophosphatidylcholine with acyl residue sum C26:1, lysophosphatidylcholine with acyl residue sum C28:0, lysophosphatidylcholine with acyl residue sum C28:1, phosphatidylcholine with diacyl residue sum C24:0, phosphatidylcholine with diacyl residue sum C28:1, phosphatidylcholine with diacyl residue sum C30:0, phosphatidylcholine with diacyl residue sum C32:0, phosphatidylcholine with diacyl residue sum C32:1, phosphatidylcholine with diacyl residue sum C32:3, phosphatidylcholine with diacyl residue sum C34:1, phosphatidylcholine with diacyl residue sum C34:2, phosphatidylcholine with diacyl residue sum C34:3, phosphatidylcholine with diacyl residue sum C34:4, phosphatidylcholine with diacyl residue sum C36:0, phosphatidylcholine with diacyl residue sum C36:1, phosphatidylcholine with diacyl residue sum C36:2, phosphatidylcholine with diacyl residue sum C36:3, phosphatidylcholine with diacyl residue sum C36:4, phosphatidylcholine with diacyl residue sum C36:5, phosphatidylcholine with diacyl residue sum C36:6, phosphatidylcholine with diacyl residue sum C38:0, phosphatidylcholine with diacyl residue sum C38:3, phosphatidylcholine with diacyl residue sum C38:4, phosphatidylcholine with diacyl residue sum C38:5, phosphatidylcholine with diacyl residue sum C38:6, phosphatidylcholine with diacyl residue sum C40:2, phosphatidylcholine with diacyl residue sum C40:3, phosphatidylcholine with diacyl residue sum C40:4, phosphatidylcholine with diacyl residue sum C40:5, phosphatidylcholine with diacyl residue sum C40:6, phosphatidylcholine with diacyl residue sum C42:0, phosphatidylcholine with diacyl residue sum C42:1, phosphatidylcholine with diacyl residue sum C42:2, phosphatidylcholine with diacyl residue sum C42:4, phosphatidylcholine with diacyl residue sum C42:5, phosphatidylcholine with diacyl residue sum C42:6, phosphatidylcholine with acyl-alkyl residue sum C30:0, phosphatidylcholine with acyl-alkyl residue sum C30:1, phosphatidylcholine with acyl-alkyl residue sum C30:2, phosphatidylcholine with acyl-alkyl residue sum C32:1, phosphatidylcholine with acyl-alkyl residue sum C32:2, phosphatidylcholine with acyl-alkyl residue sum C34:0, phosphatidylcholine with acyl-alkyl residue sum C34:1, phosphatidylcholine with acyl-alkyl residue sum C34:2, phosphatidylcholine with acyl-alkyl residue sum C34:3, phosphatidylcholine with acyl-alkyl residue sum C36:0, phosphatidylcholine with acyl-alkyl residue sum C36:1, phosphatidylcholine with acyl-alkyl residue sum C36:2, phosphatidylcholine with acyl-alkyl residue sum C36:3, phosphatidylcholine with acyl-alkyl residue sum C36:4, phosphatidylcholine with acyl-alkyl residue sum C36:5, phosphatidylcholine with acyl-alkyl residue sum C38:0, phosphatidylcholine with acyl-alkyl residue sum C38:1, phosphatidylcholine with acyl-alkyl residue sum C38:2, phosphatidylcholine with acyl-alkyl residue sum C38:3, phosphatidylcholine with acyl-alkyl residue sum C38:4, phosphatidylcholine with acyl-alkyl residue sum C38:5, phosphatidylcholine with acyl-alkyl residue sum C38:6, phosphatidylcholine with acyl-alkyl residue sum C40:1, phosphatidylcholine with acyl-alkyl residue sum C40:2, phosphatidylcholine with acyl-alkyl residue sum C40:3, phosphatidylcholine with acyl-alkyl residue sum C40:4, phosphatidylcholine with acyl-alkyl residue sum C40:5, phosphatidylcholine with acyl-alkyl residue sum C40:6, phosphatidylcholine with acyl-alkyl residue sum C42:2, phosphatidylcholine with acyl-alkyl residue sum C42:3, phosphatidylcholine with acyl-alkyl residue sum C42:5, phosphatidylcholine with acyl-alkyl residue sum C44:3, phosphatidylcholine with acyl-alkyl residue sum C44:4, phosphatidylcholine with acyl-alkyl residue sum C44:5, phosphatidylcholine with acyl-alkyl residue sum C44:6, hydroxysphingomyelin with acyl residue sum C14:1, hydroxysphingomyelin with acyl residue sum C16:1, hydroxysphingomyelin with acyl residue sum C22:1, hydroxysphingomyelin with acyl residue sum C22:2, hydroxysphingomyelin with acyl residue sum C24:1, sphingomyelin with acyl residue sum C16:0, sphingomyelin with acyl residue sum C16:1, sphingomyelin with acyl residue sum C18:0, sphingomyelin with acyl residue sum C18:1, sphingomyelin with acyl residue sum C20:2, sphingomyelin with acyl residue sum C24:0, sphingomyelin with acyl residue sum C24:1, sphingomyelin with acyl residue sum C26:0, sphingomyelin with acyl residue sum C26:1, hexoses [including glucose], alanine, arginine, asparagine, aspartate, citrulline, glutamine, glutamate, glycine, histidine, isoleucine, lysine, methionine, ornithine, phenylalanine, proline, serine, threonine, tryptophan, tyrosine, valine, asymmetric dimethylarginine, alpha-aminoadipic acid, creatinine, kynurenine, methionine sulfoxide, putrescine, sarcosine, symmetric dimethylarginine, spermidine, spermine, trans-4-hydroxyproline, or taurine.
31. A method of predicting mortality in an individual with sepsis comprising: obtaining a biological sample from the individual; measuring one or more of the following biomarkers: adrenoceptor β2 (ADRB2), CD177 molecule (CD177), carboxypeptidase vitellogenic like (CPVL), C-X3-C motif chemokine receptor 1 (CX3CR1), defensin α3 (DEFA3), Fc receptor like 5 (FCRL5), G protein subunit γ2 (GNG2), interleukin 10 receptor subunit α (IL10RA), kinesin light chain 3 (KLC3), oleoyl-ACP hydrolase (OLAH), pyruvate kinase M1/2 (PKM), radical S-adenosyl methionine domain containing 2 (RSAD2), STE20 related adaptor β (STRADB), tyrosylprotein sulfotransferase 1 (TPST1), tetraspanin 5 (TSPAN5), tetratricopeptide repeat domain 9C (TTC9C), zinc finger with KRAB and SCAN domains 1 (ZKSCAN1), C-reactive protein (CRP), C-X-C motif chemokine ligand 10/interferon gamma-induced protein 10 (CXCL10/IP-10), D-dimer, ferritins, fibrinogens, (soluble) intercellular adhesion molecule 1 (ICAM-1), interferon gamma (IFNγ), interleukin-1 receptor antagonist (IL-1RA), interleukin-5 (IL-5), interleukin-6 (IL-6), interleukin-6 receptor α (IL-6Rα), interleukin-18 (IL-18), interleukin-18-binding protein (IL-18BP), lipocalin-2 (LCN-2), matrix metalloproteinase-8 (MMP-8), procalcitonin (PCT), (soluble) receptor for advanced glycation end products (RAGE), tumor necrosis factor receptor 1 (TNF-R1), tumor necrosis factor alpha (TNFα), vascular endothelial growth factors (VEGF), von Willebrand factor A2 domain (vWF-A2), carnitine, acetylcarnitine, propionylcarnitine, malonylcarnitine, methylmalonylcarnitine, hydroxypropionylcarnitine, propenoylcarnitine, butyrylcarnitine, hydroxybutyrylcarnitine, fumarylcarnitine, valerylcarnitine, glutarylcarnitine, hydroxyvalerylcarnitine, tiglylcarnitine, hexanoylcarnitine, hydroxyhexanoylcarnitine, pimeloylcarnitine, decanoylcarnitine, decadienylcarnitine, tetradecenoylcarnitine, hydroxytetradecenoylcarnitine, hydroxytetradecadienylcarnitine, hexadecanoylcarnitine, hexadecenoylcarnitine, octadecanoylcarnitine, octadecenoylcarnitine, lysophosphatidylcholine with acyl residue sum C16:0, lysophosphatidylcholine with acyl residue sum C16:1, lysophosphatidylcholine with acyl residue sum C17:0, lysophosphatidylcholine with acyl residue sum C18:0, lysophosphatidylcholine with acyl residue sum C18:1, lysophosphatidylcholine with acyl residue sum C18:2, lysophosphatidylcholine with acyl residue sum C20:3, lysophosphatidylcholine with acyl residue sum C20:4, lysophosphatidylcholine with acyl residue sum C24:0, lysophosphatidylcholine with acyl residue sum C26:0, lysophosphatidylcholine with acyl residue sum C26:1, lysophosphatidylcholine with acyl residue sum C28:0, lysophosphatidylcholine with acyl residue sum C28:1, phosphatidylcholine with diacyl residue sum C24:0, phosphatidylcholine with diacyl residue sum C28:1, phosphatidylcholine with diacyl residue sum C30:0, phosphatidylcholine with diacyl residue sum C32:0, phosphatidylcholine with diacyl residue sum C32:1, phosphatidylcholine with diacyl residue sum C32:3, phosphatidylcholine with diacyl residue sum C34:1, phosphatidylcholine with diacyl residue sum C34:2, phosphatidylcholine with diacyl residue sum C34:3, phosphatidylcholine with diacyl residue sum C34:4, phosphatidylcholine with diacyl residue sum C36:0, phosphatidylcholine with diacyl residue sum C36:1, phosphatidylcholine with diacyl residue sum C36:2, phosphatidylcholine with diacyl residue sum C36:3, phosphatidylcholine with diacyl residue sum C36:4, phosphatidylcholine with diacyl residue sum C36:5, phosphatidylcholine with diacyl residue sum C36:6, phosphatidylcholine with diacyl residue sum C38:0, phosphatidylcholine with diacyl residue sum C38:3, phosphatidylcholine with diacyl residue sum C38:4, phosphatidylcholine with diacyl residue sum C38:5, phosphatidylcholine with diacyl residue sum C38:6, phosphatidylcholine with diacyl residue sum C40:2, phosphatidylcholine with diacyl residue sum C40:3, phosphatidylcholine with diacyl residue sum C40:4, phosphatidylcholine with diacyl residue sum C40:5, phosphatidylcholine with diacyl residue sum C40:6, phosphatidylcholine with diacyl residue sum C42:0, phosphatidylcholine with diacyl residue sum C42:1, phosphatidylcholine with diacyl residue sum C42:2, phosphatidylcholine with diacyl residue sum C42:4, phosphatidylcholine with diacyl residue sum C42:5, phosphatidylcholine with diacyl residue sum C42:6, phosphatidylcholine with acyl-alkyl residue sum C30:0, phosphatidylcholine with acyl-alkyl residue sum C30:1, phosphatidylcholine with acyl-alkyl residue sum C30:2, phosphatidylcholine with acyl-alkyl residue sum C32:1, phosphatidylcholine with acyl-alkyl residue sum C32:2, phosphatidylcholine with acyl-alkyl residue sum C34:0, phosphatidylcholine with acyl-alkyl residue sum C34:1, phosphatidylcholine with acyl-alkyl residue sum C34:2, phosphatidylcholine with acyl-alkyl residue sum C34:3, phosphatidylcholine with acyl-alkyl residue sum C36:0, phosphatidylcholine with acyl-alkyl residue sum C36:1, phosphatidylcholine with acyl-alkyl residue sum C36:2, phosphatidylcholine with acyl-alkyl residue sum C36:3, phosphatidylcholine with acyl-alkyl residue sum C36:4, phosphatidylcholine with acyl-alkyl residue sum C36:5, phosphatidylcholine with acyl-alkyl residue sum C38:0, phosphatidylcholine with acyl-alkyl residue sum C38:1, phosphatidylcholine with acyl-alkyl residue sum C38:2, phosphatidylcholine with acyl-alkyl residue sum C38:3, phosphatidylcholine with acyl-alkyl residue sum C38:4, phosphatidylcholine with acyl-alkyl residue sum C38:5, phosphatidylcholine with acyl-alkyl residue sum C38:6, phosphatidylcholine with acyl-alkyl residue sum C40:1, phosphatidylcholine with acyl-alkyl residue sum C40:2, phosphatidylcholine with acyl-alkyl residue sum C40:3, phosphatidylcholine with acyl-alkyl residue sum C40:4, phosphatidylcholine with acyl-alkyl residue sum C40:5, phosphatidylcholine with acyl-alkyl residue sum C40:6, phosphatidylcholine with acyl-alkyl residue sum C42:2, phosphatidylcholine with acyl-alkyl residue sum C42:3, phosphatidylcholine with acyl-alkyl residue sum C42:5, phosphatidylcholine with acyl-alkyl residue sum C44:3, phosphatidylcholine with acyl-alkyl residue sum C44:4, phosphatidylcholine with acyl-alkyl residue sum C44:5, phosphatidylcholine with acyl-alkyl residue sum C44:6, hydroxysphingomyelin with acyl residue sum C14:1, hydroxysphingomyelin with acyl residue sum C16:1, hydroxysphingomyelin with acyl residue sum C22:1, hydroxysphingomyelin with acyl residue sum C22:2, hydroxysphingomyelin with acyl residue sum C24:1, sphingomyelin with acyl residue sum C16:0, sphingomyelin with acyl residue sum C16:1, sphingomyelin with acyl residue sum C18:0, sphingomyelin with acyl residue sum C18:1, sphingomyelin with acyl residue sum C20:2, sphingomyelin with acyl residue sum C24:0, sphingomyelin with acyl residue sum C24:1, sphingomyelin with acyl residue sum C26:0, sphingomyelin with acyl residue sum C26:1, hexoses [including glucose], alanine, arginine, asparagine, aspartate, citrulline, glutamine, glutamate, glycine, histidine, isoleucine, lysine, methionine, ornithine, phenylalanine, proline, serine, threonine, tryptophan, tyrosine, valine, asymmetric dimethylarginine, alpha-aminoadipic acid, creatinine, kynurenine, methionine sulfoxide, putrescine, sarcosine, symmetric dimethylarginine, spermidine, spermine, trans-4-hydroxyproline, or taurine from the biological sample;
and predicting mortality in an individual with sepsis, based at least in part on levels of: adrenoceptor β2 (ADRB2), CD177 molecule (CD177), carboxypeptidase vitellogenic like (CPVL), C-X3-C motif chemokine receptor 1 (CX3CR1), defensin α3 (DEFA3), Fc receptor like 5 (FCRL5), G protein subunit γ2 (GNG2), interleukin 10 receptor subunit α (IL10RA), kinesin light chain 3 (KLC3), oleoyl-ACP hydrolase (OLAH), pyruvate kinase M1/2 (PKM), radical S-adenosyl methionine domain containing 2 (RSAD2), STE20 related adaptor β (STRADB), tyrosylprotein sulfotransferase 1 (TPST1), tetraspanin 5 (TSPAN5), tetratricopeptide repeat domain 9C (TTC9C), zinc finger with KRAB and SCAN domains 1 (ZKSCAN1), C-reactive protein (CRP), C-X-C motif chemokine ligand 10/interferon gamma-induced protein 10 (CXCL10/IP-10), D-dimer, ferritins, fibrinogens, (soluble) intercellular adhesion molecule 1 (ICAM-1), interferon gamma (IFNγ), interleukin-1 receptor antagonist (IL-1RA), interleukin-5 (IL-5), interleukin-6 (IL-6), interleukin-6 receptor α (IL-6Rα), interleukin-18 (IL-18), interleukin-18-binding protein (IL-18BP), lipocalin-2 (LCN-2), matrix metalloproteinase-8 (MMP-8), procalcitonin (PCT), (soluble) receptor for advanced glycation end products (RAGE), tumor necrosis factor receptor 1 (TNF-R1), tumor necrosis factor alpha (TNFα), vascular endothelial growth factors (VEGF), von Willebrand factor A2 domain (vWF-A2), carnitine, acetylcarnitine, propionylcarnitine, malonylcarnitine, methylmalonylcarnitine, hydroxypropionylcarnitine, propenoylcarnitine, butyrylcarnitine, hydroxybutyrylcarnitine, fumarylcarnitine, valerylcarnitine, glutarylcarnitine, hydroxyvalerylcarnitine, tiglylcarnitine, hexanoylcarnitine, hydroxyhexanoylcarnitine, pimeloylcarnitine, decanoylcarnitine, decadienylcarnitine, tetradecenoylcarnitine, hydroxytetradecenoylcarnitine, hydroxytetradecadienylcarnitine, hexadecanoylcarnitine, hexadecenoylcarnitine, octadecanoylcarnitine, octadecenoylcarnitine, lysophosphatidylcholine with acyl residue sum C16:0, lysophosphatidylcholine with acyl residue sum C16:1, lysophosphatidylcholine with acyl residue sum C17:0, lysophosphatidylcholine with acyl residue sum C18:0, lysophosphatidylcholine with acyl residue sum C18:1, lysophosphatidylcholine with acyl residue sum C18:2, lysophosphatidylcholine with acyl residue sum C20:3, lysophosphatidylcholine with acyl residue sum C20:4, lysophosphatidylcholine with acyl residue sum C24:0, lysophosphatidylcholine with acyl residue sum C26:0, lysophosphatidylcholine with acyl residue sum C26:1, lysophosphatidylcholine with acyl residue sum C28:0, lysophosphatidylcholine with acyl residue sum C28:1, phosphatidylcholine with diacyl residue sum C24:0, phosphatidylcholine with diacyl residue sum C28:1, phosphatidylcholine with diacyl residue sum C30:0, phosphatidylcholine with diacyl residue sum C32:0, phosphatidylcholine with diacyl residue sum C32:1, phosphatidylcholine with diacyl residue sum C32:3, phosphatidylcholine with diacyl residue sum C34:1, phosphatidylcholine with diacyl residue sum C34:2, phosphatidylcholine with diacyl residue sum C34:3, phosphatidylcholine with diacyl residue sum C34:4, phosphatidylcholine with diacyl residue sum C36:0, phosphatidylcholine with diacyl residue sum C36:1, phosphatidylcholine with diacyl residue sum C36:2, phosphatidylcholine with diacyl residue sum C36:3, phosphatidylcholine with diacyl residue sum C36:4, phosphatidylcholine with diacyl residue sum C36:5, phosphatidylcholine with diacyl residue sum C36:6, phosphatidylcholine with diacyl residue sum C38:0, phosphatidylcholine with diacyl residue sum C38:3, phosphatidylcholine with diacyl residue sum C38:4, phosphatidylcholine with diacyl residue sum C38:5, phosphatidylcholine with diacyl residue sum C38:6, phosphatidylcholine with diacyl residue sum C40:2, phosphatidylcholine with diacyl residue sum C40:3, phosphatidylcholine with diacyl residue sum C40:4, phosphatidylcholine with diacyl residue sum C40:5, phosphatidylcholine with diacyl residue sum C40:6, phosphatidylcholine with diacyl residue sum C42:0, phosphatidylcholine with diacyl residue sum C42:1, phosphatidylcholine with diacyl residue sum C42:2, phosphatidylcholine with diacyl residue sum C42:4, phosphatidylcholine with diacyl residue sum C42:5, phosphatidylcholine with diacyl residue sum C42:6, phosphatidylcholine with acyl-alkyl residue sum C30:0, phosphatidylcholine with acyl-alkyl residue sum C30:1, phosphatidylcholine with acyl-alkyl residue sum C30:2, phosphatidylcholine with acyl-alkyl residue sum C32:1, phosphatidylcholine with acyl-alkyl residue sum C32:2, phosphatidylcholine with acyl-alkyl residue sum C34:0, phosphatidylcholine with acyl-alkyl residue sum C34:1, phosphatidylcholine with acyl-alkyl residue sum C34:2, phosphatidylcholine with acyl-alkyl residue sum C34:3, phosphatidylcholine with acyl-alkyl residue sum C36:0, phosphatidylcholine with acyl-alkyl residue sum C36:1, phosphatidylcholine with acyl-alkyl residue sum C36:2, phosphatidylcholine with acyl-alkyl residue sum C36:3, phosphatidylcholine with acyl-alkyl residue sum C36:4, phosphatidylcholine with acyl-alkyl residue sum C36:5, phosphatidylcholine with acyl-alkyl residue sum C38:0, phosphatidylcholine with acyl-alkyl residue sum C38:1, phosphatidylcholine with acyl-alkyl residue sum C38:2, phosphatidylcholine with acyl-alkyl residue sum C38:3, phosphatidylcholine with acyl-alkyl residue sum C38:4, phosphatidylcholine with acyl-alkyl residue sum C38:5, phosphatidylcholine with acyl-alkyl residue sum C38:6, phosphatidylcholine with acyl-alkyl residue sum C40:1, phosphatidylcholine with acyl-alkyl residue sum C40:2, phosphatidylcholine with acyl-alkyl residue sum C40:3, phosphatidylcholine with acyl-alkyl residue sum C40:4, phosphatidylcholine with acyl-alkyl residue sum C40:5, phosphatidylcholine with acyl-alkyl residue sum C40:6, phosphatidylcholine with acyl-alkyl residue sum C42:2, phosphatidylcholine with acyl-alkyl residue sum C42:3, phosphatidylcholine with acyl-alkyl residue sum C42:5, phosphatidylcholine with acyl-alkyl residue sum C44:3, phosphatidylcholine with acyl-alkyl residue sum C44:4, phosphatidylcholine with acyl-alkyl residue sum C44:5, phosphatidylcholine with acyl-alkyl residue sum C44:6, hydroxysphingomyelin with acyl residue sum C14:1, hydroxysphingomyelin with acyl residue sum C16:1, hydroxysphingomyelin with acyl residue sum C22:1, hydroxysphingomyelin with acyl residue sum C22:2, hydroxysphingomyelin with acyl residue sum C24:1, sphingomyelin with acyl residue sum C16:0, sphingomyelin with acyl residue sum C16:1, sphingomyelin with acyl residue sum C18:0, sphingomyelin with acyl residue sum C18:1, sphingomyelin with acyl residue sum C20:2, sphingomyelin with acyl residue sum C24:0, sphingomyelin with acyl residue sum C24:1, sphingomyelin with acyl residue sum C26:0, sphingomyelin with acyl residue sum C26:1, hexoses [including glucose], alanine, arginine, asparagine, aspartate, citrulline, glutamine, glutamate, glycine, histidine, isoleucine, lysine, methionine, ornithine, phenylalanine, proline, serine, threonine, tryptophan, tyrosine, valine, asymmetric dimethylarginine, alpha-aminoadipic acid, creatinine, kynurenine, methionine sulfoxide, putrescine, sarcosine, symmetric dimethylarginine, spermidine, spermine, trans-4-hydroxyproline, or taurine.
32. The method of anyone of embodiments 1-19 or 31, the system of any one of embodiments 22-25, or the array of embodiments 27-30, wherein the method further comprises treating the individual for sepsis, preventing the development of sepsis in the individual, or ameliorating the symptoms of sepsis in the individual, and wherein the system or array is used to treat the individual for sepsis, prevent the development of sepsis in the individual, or ameliorating the symptoms of sepsis in the individual.

### EXAMPLES

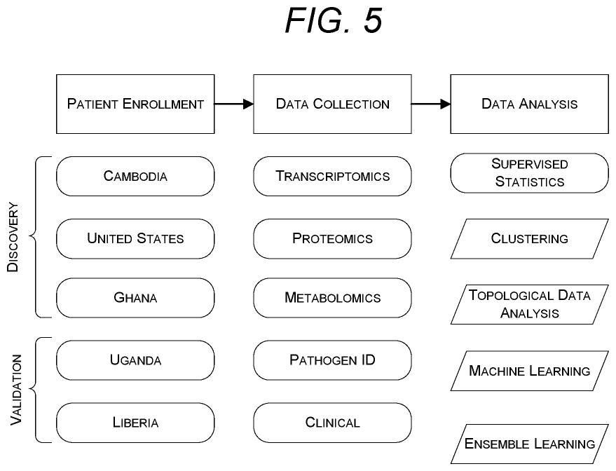

Example 1: The Austere environments Consortium for Enhanced Sepsis Outcomes (ACESO) follows a multi-omics systems biology approach for profiling sepsis patients into disease-response phenotypes, which informs the development of robust and accurate host-biomarker panels for sepsis diagnosis and prognosis (FIG. 5). The aim of this study was to use topological data analysis (TDA) to identify gene and protein expression phenotypes in sepsis patients enrolled in an ACESO observational study from sites in Cambodia, Ghana and the USA.

Concentrations of 48 proteins representing a range of biologic pathways were measured by Luminex multiplex immunoassay in peripheral blood samples from 586 sepsis patients. In addition, RNA sequencing was performed on 506 patients from the same cohort, and the 1000 protein-coding genes with the largest standard deviation were selected for analysis. Topological data analysis (TDA) was used as an unsupervised method for identifying clusters of patients with similar gene or protein expression profiles (molecular phenotypes), as well as broader trends across the TDA network. In addition, differences in demographic, clinical and basic laboratory measurements between TDA clusters were tested for statistical significance to inform on sepsis endotypes associated with the gene and protein expression phenotypes.

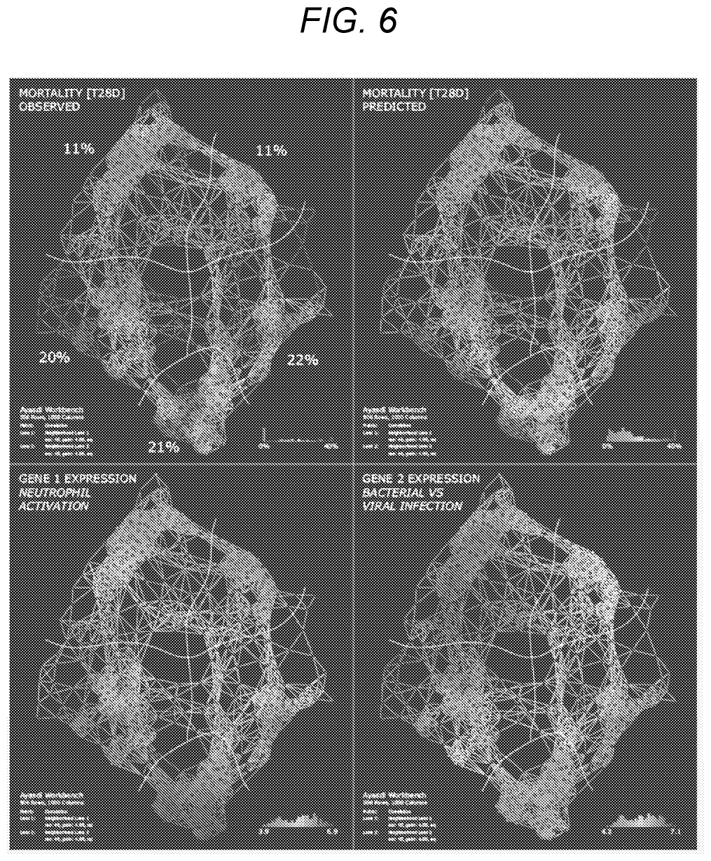

TDA networks of gene expression in the ACESO discovery cohort (n=506) show the heterogeneity of sepsis in an unsupervised, data-driven manner. In TDA a 2- dimensional topological network is created which is based on the similarity between data points, as well as the overall distribution of the data in n-dimensional space. Nodes represent groups of patients with shared characteristics, whereas edges (lines) indicate that one or more patients are shared between two nodes. Any (meta)data available for the same patient set can be used to generate a gray-scale overlay (average values are calculated for each node) (FIG. 6).

TDA analysis distinguished 5 distinct sepsis phenotypes based on gene expression, with significantly different levels of mortality (at 28 days post-enrollment). Using feature selection and machine learning a set of 13 genes was identified for predicting mortality in the discovery cohort (top right of FIG. 6) with a sensitivity of 90-96% for the high-mortality TDA groups. Furthermore, the distributions of genes across the TDA network highlights biological pathways relevant to the different sepsis phenotypes.

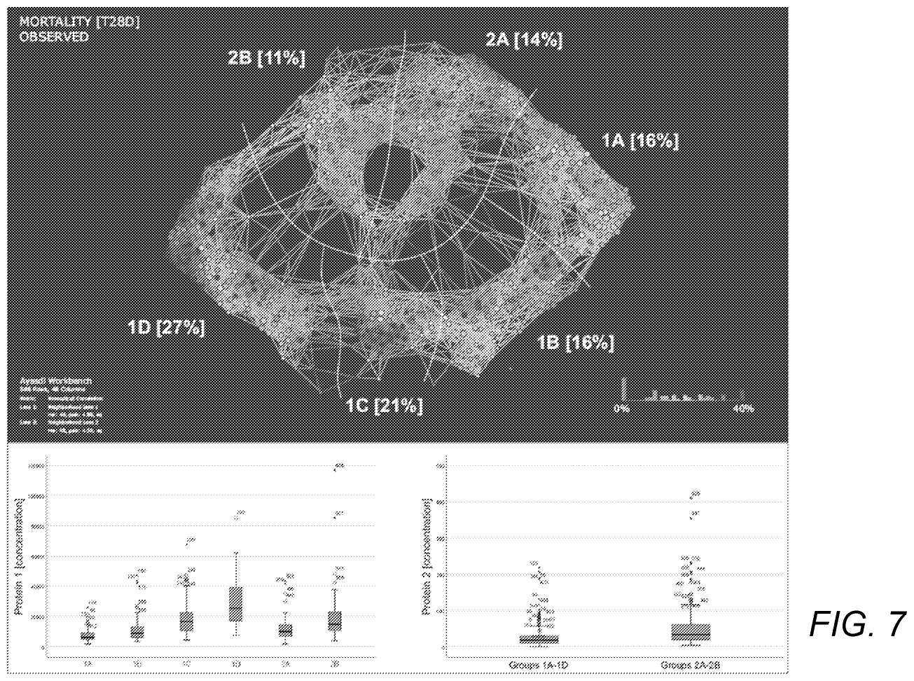

TDA networks of protein expression in the ACESO discovery cohort indicated two major trends within the protein data and identified six overlapping patient clusters. Four of these clusters, comprising two-thirds of the study cohort, form a continuous spectrum along the primary axis of the network. Protein concentrations along this spectrum are predictive of mortality risk within the first 28 days of disease, representing a two-fold increase in risk between patients, independent of site of enrollment, at either end of the spectrum. In addition, there are significant differences between these phenotypes in terms of clinical presentation, laboratory measurements and blood cell counts (FIG. 7).

Example 2: Sepsis is a major risk factor in patients with COVID-19, and those who go on to develop (severe) sepsis and require hospital or intensive care unit admission have poorer outcomes (mortality and long-term morbidity). Host biomarker data were collected from a cohort of COVID-19 patients in order to elucidate their role in the pathogenesis of this disease, as well as to assess the feasibility of using host biomarker levels to prognose disease severity and long-term (post 90-day) morbidity in COVID-19 patients. Baseline levels of 15 cytokines were measured in peripheral blood samples using the Ella multiplex assay. In addition, a wide range of demographic, clinical and laboratory variables were collected

Exploratory analyses were performed to understand the associations between blood cytokine levels, different demographics (e.g., age, sex, race), clinical parameters (e.g., pre-existing conditions, laboratory measurements, vital signs) and clinical outcomes, primarily as risk of hospitalization. Positive associations were generally found between blood cytokine levels and the risk of hospitalization, and ensemble machine learning methods were used to find optimal cut-offs in the associations between host biomarkers levels and hospitalization risk.

The ensemble machine learning algorithm was a combination of random forest (RF) and classification and regression tree (CART), along with the extreme gradient boosting. 10,000 simulations of the model were performed in order to minimize or account for the two common sources of uncertainty in prediction models: (1) the errors introduced by the use of imperfect initial conditions, and (2) errors introduced due to imperfections in the model formulation. The accuracies of the individual models (RF and CART) were assessed prior to creating the ensemble model based on a combination of the two. The ensemble model showed 20% higher accuracy compared with either of these individual methods. The area under the curve (AUC) for the training dataset was 0.88 and for testing set was to 0.83.

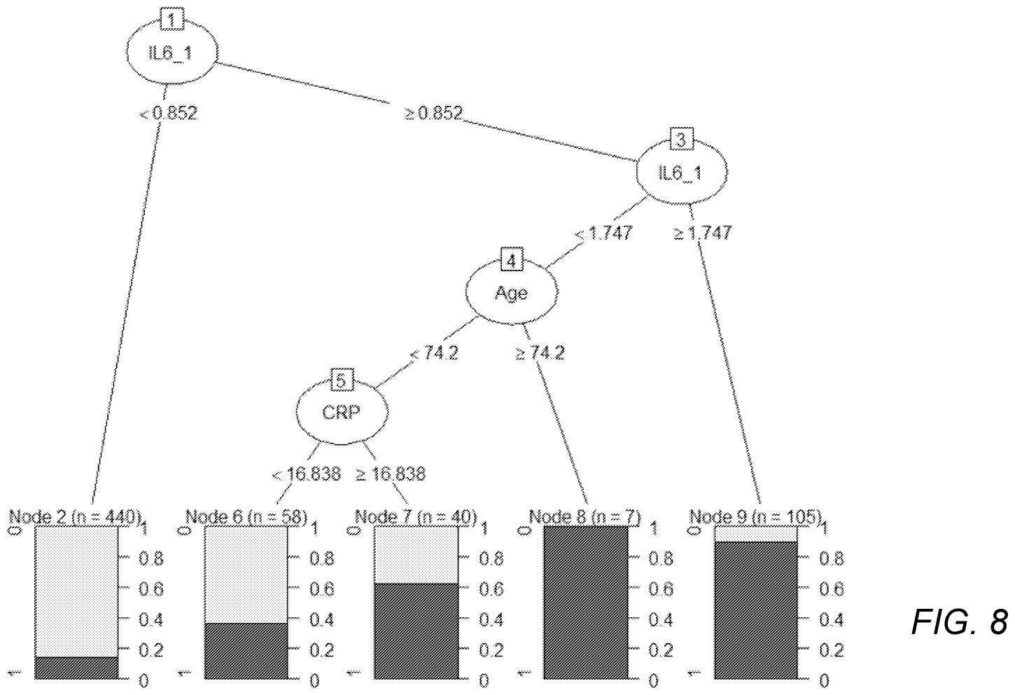

FIG. 8 depicts the CART tree based on the ensemble model. Among patients diagnosed with COVID-19, those who had IL-6 values less than 2.34 pg/ml were 87% more likely to not require hospitalization (NODE 2). In contrast, patients with IL-6 levels between 2.34 pg/ml and 5.74 pg/ml and who were also older than 74 had a very high likelihood of requiring hospitalization (NODE 8), and the same was true for any patients with IL-6 levels equal to or greater than 5.74 pg/ml (NODE 9). For patients with IL-6 levels between 2.34 pg/ml and 5.74 pg/ml but who were less than 74 years old the CRP levels were relevant. Those patients with CRP levels below 16.84 pg/ml were more likely to not require hospitalization (NODE 6)., whereas those with CRP levels above 16.84 were 60% more likely to require hospitalization (NODE 7). Finally, these cut-offs still need validation in an external cohort of COVID-19 patients.

All publications, patents and patent applications cited in this specification are incorporated herein by reference in their entireties as if each individual publication, patent or patent application were specifically and individually indicated to be incorporated by reference. While the foregoing has been described in terms of various embodiments, the skilled artisan will appreciate that various modifications, substitutions, omissions, and changes may be made without departing from the spirit thereof.

What is claimed is:

1. A method of generating a model predicting severe disease in an individual with sepsis or at risk of developing sepsis comprising: generating a discovery database storing first values of a plurality of clinical parameters and clinical outcomes associated with a plurality of first subjects; executing a plurality of data quality control algorithms to select a subset of clinical parameters from the plurality of clinical parameters; executing topological data analysis and/or clustering for the plurality of subsets of clinical parameters; executing a plurality of feature selection machine learning and/or ensemble learning models based on a plurality of classification and/or time-to-event analysis algorithms; and outputting a model for predicting severe disease in the individual with sepsis or at risk of developing sepsis.

2. The method of claim 1, further comprising pre-processing data that is stored in the discovery database, including: determining that a first value of at least one of the plurality of clinical parameters is missing; estimating a reference value for the at least one of the plurality of clinical parameters that is missing; and storing the reference value as the first value of the at least one of the plurality of clinical parameters in the discovery database.

3. The method of claim 1 or 2, wherein the plurality of data quality control algorithms comprise at least one of: differential expression algorithms, principal component analysis, k-nearest neighbor imputation algorithms, three-sigma rule algorithms, and empirical Bayes method algorithms.

4. The method of any one of claims 1-3, wherein the topological data analysis groups individuals or samples based on similarities in the plurality of subsets of clinical parameters, as well as the algebraic topology of the same data, wherein clusters are delineated based on the persistence homology of the node density and connectivity, and wherein sepsis response phenotypes are defined based on the identified clusters.

5. The method of any one of claims 1-3, wherein the cluster analysis discretizes the plurality of subsets of clinical parameters based on measures of similarity, and wherein sepsis response phenotypes are defined based on the identified clusters.

6. The method of any one of claims 1-5, wherein the feature selection machine learning models comprise at least one of: unsupervised machine learning algorithm, supervised machine learning algorithm, minimum redundancy maximum relevance, Student's t-test, Mann-Whitney U test, random forest, logistic regression, or neural networks.

7. The method of any one of claims 1-5, wherein the feature selection ensemble learning models comprise at least one of: cluster analysis, unsupervised machine learning algorithm, supervised machine learning algorithm, minimum redundancy maximum relevance, Student's t-test, Mann-Whitney U test, random forest, logistic regression, neural networks, Bayes optimal classifier, classification and regression tree, bootstrap aggregating, boosting, Bayesian model averaging, Bayesian model combination, a bucket of models, or stacking.

8. The method of any one of claims 1-7, wherein the plurality of clinical parameters comprise one or more nucleic acid data markers, one or more protein data markers, one or more metabolite data markers, one or more clinical outcomes data, one or more administrative health data, or a combination thereof.

9. The method of claim 8, wherein the nucleic acid data markers comprise one or more of: level of adhesion G protein-coupled receptor E1 (ADGRE1) in a sample from the individual, level of adrenoceptor β2 (ADRB2) in a sample from the individual, level of angiotensin II receptor associated protein (AGTRAP) in a sample from the individual, level of AKT serine/threonine kinase 1 (AKT1) in a sample from the individual, level of 5′-aminolevulinate synthase 2 (ALAS2) in a sample from the individual, level of alkaline phosphatase, biomineralization associated (ALPL) in a sample from the individual, level of ankyrin repeat domain 22 (ANKRD22) in a sample from the individual, level of annexin A3 (ANXA3) in a sample from the individual, level of arginase 1 (ARG1) in a sample from the individual, level of BCL2 like 1 (BCL2L1) in a sample from the individual, level of BMX non-receptor tyrosine kinase (BMX) in a sample from the individual, level of chromosome 6 open reading frame 62 (C6orf62) in a sample from the individual, level of carbonic anhydrase 2 (CA2) in a sample from the individual, level of C-C motif chemokine ligand 5 (CCL5) in a sample from the individual, level of C-C motif chemokine receptor 3 (CCR3) in a sample from the individual, level of CD4 molecule (CD4) in a sample from the individual, level of CD24 molecule (CD24) in a sample from the individual, level of CD177 molecule (CD177) in a sample from the individual, level of CD274 molecule (CD274) in a sample from the individual, level of cell division cycle 34, ubiqiutin conjugating enzyme (CDC34) in a sample from the individual, level of complement factor D (CFD) in a sample from the individual, level of chitinase 3 like 1 (CHI3L1) in a sample from the individual, level of carbohydrate sulfotransferase 2 (CHST2) in a sample from the individual, level of C-type lectin domain family 4 member E (CLEC4E) in a sample from the individual, level of cytidine/uridine monophosphate kinase 2 (CMPK2) in a sample from the individual, level of cytochrome C oxidase assembly factor 1 homolog (COA1) in a sample from the individual, level of carnitine palmitoyltransferase 1A (CPT1A) in a sample from the individual, level of carboxypeptidase vitellogenic like (CPVL) in a sample from the individual, level of chondroitin sulfate N-acetylgalactosaminyltransferase 1 (CSGALNACT1) in a sample from the individual, level of cystatin C (CST3) in a sample from the individual, level of C-X3-C motif chemokine receptor 1 (CX3CR1) in a sample from the individual, level of DNA damage inducible transcript 4 (DDIT4) in a sample from the individual, level of defensin α3 (DEFA3) in a sample from the individual, level of defensin α4 (DEFA4) in a sample from the individual, level of DNA J heat shock protein family (Hsp40) member C1 (DNAJC1) in a sample from the individual, level of DNA damage regulated autophagy modulator 1 (DRAM1) in a sample from the individual, level of deoxyuridine triphosphatase (DUT) in a sample from the individual, level of dual specificity tyrosine phosphorylation regulated kinase 3 (DYRK3) in a sample from the individual, level of erythrocyte membrane protein band 4.2 (EPB42) in a sample from the individual, level of family with sequence similarity 174 member C (FAM174C) in a sample from the individual, level of F-box and WD repeat domain containing 2 (FBXW2) in a sample from the individual, level of Fc receptor like 5 (FCRL5) in a sample from the individual, level of ferrochelatase (FECH) in a sample from the individual, level of fibroblast growth factor binding protein 2 (FGFBP2) in a sample from the individual, level of Fms related receptor tyrosine kinase 3 (FLT3) in a sample from the individual, level of formyl peptide receptor 1 (FPR1) in a sample from the individual, level of GATA binding protein 1 (GATA1) in a sample from the individual, level of GTPase, IMAP family member 4 (GIMAP4) in a sample from the individual, level of GTPase, IMAP family member 7 (GIMAP7) in a sample from the individual, level of GTPase, IMAP family member 8 (GIMAP8) in a sample from the individual, level of G protein subunit γ2 (GNG2) in a sample from the individual, level of granulysin (GNLY) in a sample from the individual, level of G protein-coupled receptor 65 (GPR65) in a sample from the individual, level of growth factor receptor bound protein 10 (GRB10) in a sample from the individual, level of glutathione S-transferase κ1 (GSTK1) in a sample from the individual, level of H3 histone pseudogene 6 (H3F3AP4) in a sample from the individual, level of hemoglobin subunit α2 (HBA2) in a sample from the individual, level of hemogen (HEMGN) in a sample from the individual, level of HECT and RLD domain containing E3 ubiquitin protein ligase family member 6 (HERC6) in a sample from the individual, level of H3.2 histone [putative] (HIST2H3PS2) in a sample from the individual, level of major histocompatibility complex, class I, B (HLA-B) in a sample from the individual, level of major histocompatibility complex, class II, DQ β1 (HLA-DQB1) in a sample from the individual, level of high mobility group box 2 (HMGB2) in a sample from the individual, level of 15-hydroxyprostaglandin dehydrogenase (HPGD) in a sample from the individual, level of hydrogen voltage gated channel 1 (HVCN1) in a sample from the individual, level of isoamyl acetate hydrolyzing esterase 1 [putative] (IAH1) in a sample from the individual, level of intercellular adhesion molecule 1 (ICAM1) in a sample from the individual, level of immediate early response 5 (IER5) in a sample from the individual, level of interferon α inducible protein 6 (IFI6) in a sample from the individual, level of interferon α inducible protein 27 (IFI27) in a sample from the individual, level of interferon induced protein 44 (IFI44) in a sample from the individual, level of interferon induced protein with tetratricopeptide repeats 1 (IFIT1) in a sample from the individual, level of interferon induced protein with tetratricopeptide repeats 2 (IFIT2) in a sample from the individual, level of interleukin 1β (IL1B) in a sample from the individual, level of interleukin 1 receptor type 1 (IL1RA) in a sample from the individual, level of interleukin 1 receptor type 2 (IL1R2) in a sample from the individual, level of interleukin 10 receptor subunit α (IL10RA) in a sample from the individual, level of interaction protein for cytohesin exchange factors 1 (IPCEF1) in a sample from the individual, level of interferon regulatory factor 2 binding protein 2 (IRF2BP2) in a sample from the individual, level of ISG15 ubiquitin like modifier (ISG15) in a sample from the individual, level of JUN proto-oncogene, AP-1 transcription factor subunit (JUN) in a sample from the individual, level of potassium voltage-gated channel subfamily E regulatory subunit 1 (KCNE1) in a sample from the individual, level of kinesin light chain 3 (KLC3) in a sample from the individual, level of kelch like family member 24 (KLHL24) in a sample from the individual, level of kringle containing transmembrane protein 1 (KREMEN1) in a sample from the individual, level of long intergenic non-protein coding RNA 861 (LINC00861) in a sample from the individual, level of lymphocyte antigen 6 family member E (LY6E) in a sample from the individual, level of MAPK associated protein 1 (MAPKAP1) in a sample from the individual, level of mediator complex subunit 28 (MED28) in a sample from the individual, level of MicroRNA 6724-4 (MIR6724-4) in a sample from the individual, level of matrix metallopeptidase 8 (MMP8) in a sample from the individual, level of multimerin 1 (MMRN1) in a sample from the individual, level of myeloperoxidase (MPO) in a sample from the individual, level of mannose receptor C type 2 (MRC2) in a sample from the individual, level of mitochondrially encoded 12S rRNA (MT-RNR1) in a sample from the individual, level of MX dynamin like GTPase 2 (MX2) in a sample from the individual, level of nuclear factor, erythroid 2 like 3 (NFE2L3) in a sample from the individual, level of 2′-5′-oligoadenylate synthetase 3 (OAS3) in a sample from the individual, level of oleoyl-ACP hydrolase (OLAH) in a sample from the individual, level of olfactomedin 4 (OLFM4) in a sample from the individual, level of peptidase inhibitor 3 (PI3) in a sample from the individual, level of phosphatidylinositol-4,5-bisphosphate 3-kinase catalytic subunit β (PIK3CB) in a sample from the individual, level of PITH domain containing 1 (PITHD1) in a sample from the individual, level of pyruvate kinase M1/2 (PKM) in a sample from the individual, level of perilipin 2 (PLIN2) in a sample from the individual, level of DNA polymerase δ interacting protein 3 (POLDIP3) in a sample from the individual, level of RAL GTPase activating protein catalytic subunit α2 (RALGAPA2) in a sample from the individual, level of RAN binding protein 9 (RANBP9) in a sample from the individual, level of REST corepressor 1 (RCOR1) in a sample from the individual, level of Rh associated glycoprotein (RHAG) in a sample from the individual, level of RNA, U1 small nuclear 2 (RNU1-2) in a sample from the individual, level of RNA, U1 small nuclear 4 (RNU1-4) in a sample from the individual, level of ribosomal protein L37a (RPL37A) in a sample from the individual, level of ribosomal protein L38 (RPL38) in a sample from the individual, level of ribosomal protein S11 (RPS11) in a sample from the individual, level of ribosomal protein S18 (RPS18) in a sample from the individual, level of radical S-adenosyl methionine domain containing 2 (RSAD2) in a sample from the individual, level of S100 calcium binding protein A8 (S100A8) in a sample from the individual, level of S100 calcium binding protein A9 (S100A9) in a sample from the individual, level of S100 calcium binding protein A12 (S100A12) in a sample from the individual, level of SAM domain, SH3 domain and nuclear localization signals 1 (SAMSN1) in a sample from the individual, level of Sin3A associated protein 30 (SAP30) in a sample from the individual, level of strawberry notch homolog 1 (SBNO1) in a sample from the individual, level of selenium binding protein 1 (SELENBP1) in a sample from the individual, level of sialic acid binding Ig like lectin 10 (SIGLEC10) in a sample from the individual, level of solute carrier family 25 member 6 (SLC25A6) in a sample from the individual, level of solute carrier family 25 member 39 (SLC25A39) in a sample from the individual, level of solute carrier family 39 member 8 (SLC39A8) in a sample from the individual, level of solute carrier family 4 member 1 [Diego Blood Group] (SLC4A1) in a sample from the individual, level of synuclein α (SNCA) in a sample from the individual, level of small nucleolar RNA, H/ACA box 44 (SNORA44) in a sample from the individual, level of superoxide dismutase 2 (SOD2) in a sample from the individual, level of spectrin α, erythrocytic 1 (SPTA1) in a sample from the individual, level of STE20 related adaptor β (STRADB) in a sample from the individual, level of syntaxin 6 (STX6) in a sample from the individual, level of switching B cell complex subunit SWAP70 (SWAP70) in a sample from the individual, level of spectrin repeat containing nuclear envelope protein 2 (SYNE2) in a sample from the individual, level of T-box transcription factor 21 (TBX21) in a sample from the individual, level of TRAF interacting protein with forkhead associated domain (TIFA) in a sample from the individual, level of toll like receptor 7 (TLR7) in a sample from the individual, level of transmembrane and coiled-coil domain family 2 (TMCC2) in a sample from the individual, level of transmembrane protein 35B (TMEM35B) in a sample from the individual, level of transmembrane protein 273 (TMEM273) in a sample from the individual, level of thymosin β10 (TMSB10) in a sample from the individual, level of TNF α induced protein 6 (TNFAIP6) in a sample from the individual, level of tyrosylprotein sulfotransferase 1 (TPST1) in a sample from the individual, level of tripartite motif containing 4 (TRIM4) in a sample from the individual, level of tetraspanin 5 (TSPAN5) in a sample from the individual, level of tetratricopeptide repeat domain 9C (TTC9C) in a sample from the individual, level of ubiquitin protein ligase E3 component N-recognin 5 (UBR5) in a sample from the individual, level of UNC-93 homolog B1, TLR signaling regulator (UNC93B1) in a sample from the individual, level of WASH complex subunit 2C (WASHC2C) in a sample from the individual, level of XIAP associated factor 1 (XAF1) in a sample from the individual, level of tyrosine 3-monooxygenase/tryptophan 5-monooxygenase activation protein E (YWHAH) in a sample from the individual, or level of or zinc finger With KRAB and SCAN domains 1 (ZKSCAN1) in a sample from the individual;
wherein the protein data markers comprise one or more of: level of a disintegrin and metalloproteinase with thrombospondin motifs 13 (ADAMTS13) in a sample from the individual, level of Angiopoietin-1 (ANGPT1) in a sample from the individual, level of Angiopoietin-2 (ANGPT2) in a sample from the individual, level of C-C chemokine receptor ligand 2/monocyte chemoattractant protein 1 (CCL2/MCP-1) in a sample from the individual, level of C-C chemokine receptor ligand 3/macrophage inflammatory protein 1-alpha (CCL3/MIP-1-α) in a sample from the individual, level of C-C chemokine receptor ligand 5/regulated on activation, normal T cell expressed and secreted (CCL5/RANTES) in a sample from the individual, level of cluster of differentiation 163 (CD163) in a sample from the individual, level of cluster of differentiation 40 ligand (CD40L) in a sample from the individual, level of chitinase-3-like protein 1 (CHI3L1) in a sample from the individual, level of C-reactive protein (CRP) in a sample from the individual, level of C-X-C motif chemokine ligand 10/interferon gamma-induced protein 10 (CXCL10/IP-10) in a sample from the individual, level of decoy receptor 3 (Dcr3) in a sample from the individual, level of D-dimer in a sample from the individual, level of E-selectin (SELE) in a sample from the individual, level of endoglin (ENG) in a sample from the individual, level of fas receptor (FAS) in a sample from the individual, level of ferritins in a sample from the individual, level of fibrinogens in a sample from the individual, level of granulocyte colony-stimulating factor (G-CSF) in a sample from the individual, level of granulocyte-macrophage colony-stimulating factor (GM-CSF) in a sample from the individual, level of (soluble) intercellular adhesion molecule 1 (ICAM-1) in a sample from the individual, level of interferon gamma (IFNγ) in a sample from the individual, level of interleukin 1 beta (IL-1β) in a sample from the individual, level of interleukin-1 receptor antagonist (IL-1RA) in a sample from the individual, level of (soluble) interleukin-2 receptor α (IL-2Rα) in a sample from the individual, level of interleukin-4 (IL-4) in a sample from the individual, level of interleukin-5 (IL-5) in a sample from the individual, level of interleukin-6 (IL-6) in a sample from the individual, level of interleukin-6 receptor α (IL-6Rα) in a sample from the individual, level of interleukin-7 (IL-7) in a sample from the individual, level of interleukin-8 (IL-8) in a sample from the individual, level of interleukin-10 (IL-10) in a sample from the individual, level of interleukin-12 ‘p70’ (IL-12 p70) in a sample from the individual, level of interleukin-15 (IL-15) in a sample from the individual, level of interleukin-16 (IL-16) in a sample from the individual, level of interleukin-17A (IL-17A) in a sample from the individual, level of interleukin-18 (IL-18) in a sample from the individual, level of interleukin-18-binding protein (IL-18BP) in a sample from the individual, level of interleukin-22 (IL-22) in a sample from the individual, level of interleukin-27 (IL-27) in a sample from the individual, level of lipocalin-2 (LCN-2) in a sample from the individual, level of matrix metalloproteinase-8 (MMP-8) in a sample from the individual, level of matrix metalloproteinase-9 (MMP-9) in a sample from the individual, level of matrix metalloproteinase-10 (MMP-10) in a sample from the individual, level of (soluble) macrophage mannose receptors in a sample from the individual, level of procalcitonin (PCT) in a sample from the individual, level of (soluble) programmed death-ligand 1 (PD-L1) in a sample from the individual, level of pentraxin 3 (PTX3) in a sample from the individual, level of (soluble) receptor for advanced glycation end products (RAGE) in a sample from the individual, level of resistin (RETN) in a sample from the individual, level of serum amyloid A proteins (SAA) in a sample from the individual, level of tyrosine kinase with immunoglobulin-like and EGF-like domains 1 (TIE1) in a sample from the individual, level of tyrosine kinase with immunoglobulin-like and EGF-like domains 2 (TIE2) in a sample from the individual, level of tissue inhibitor of metalloproteinases 1 (TIMP1) in a sample from the individual, level of tissue inhibitor of metalloproteinases 2 (TIMP2) in a sample from the individual, level of tissue inhibitor of metalloproteinases 3 (TIMP3) in a sample from the individual, level of tissue inhibitor of metalloproteinases 4 (TIMP4) in a sample from the individual, level of tumor necrosis factor receptor 1 (TNF-R1) in a sample from the individual, level of tumor necrosis factor alpha (TNFα) in a sample from the individual, level of tissue plasminogen activator (tPA) in a sample from the individual, level of tissue plasminogen activator inhibitor 1 (tPAI-1) in a sample from the individual, level of TNF-related apoptosis-inducing ligand (TRAIL) in a sample from the individual, level of (soluble) triggering receptor expressed on myeloid cells 1 (TREM1) in a sample from the individual, level of urokinase receptor (uPar) in a sample from the individual, level of (soluble) vascular cell adhesion molecule 1 (VCAM-1) in a sample from the individual, level of vascular endothelial growth factors (VEGF) in a sample from the individual, level of (soluble) vascular endothelial growth factor receptor 1 (VEGFR-1) in a sample from the individual, level of (soluble) vascular endothelial growth factor receptor 2 (VEGFR-2) in a sample from the individual, or level of von Willebrand factor A2 domain (vWF-A2) in a sample from the individual;
wherein the metabolite data comprise one or more of: levels of fatty acyls and their constituent molecular species in a sample from the individual, levels of glycerolipids and their constituent molecular species in a sample from the individual, levels of glycerophospholipids and their constituent molecular species in a sample from the individual, levels of sphingolipids and their constituent molecular species in a sample from the individual, levels of sterol lipids and their constituent molecular species in a sample from the individual, levels of prenol lipids and their constituent molecular species in a sample from the individual, levels of saccharolipids and their constituent molecular species in a sample from the individual, levels of polyketides and their constituent molecular species in a sample from the individual, levels of carbohydrates and their constituent molecular species in a sample from the individual, levels of organic acids and their derivatives and constituent molecular species in a sample from the individual, levels of organo-heterocyclic compounds and their constituent molecular species in a sample from the individual, levels of organo-oxygen compounds and their constituent molecular species in a sample from the individual, levels of organo-nitrogen compounds and their constituent molecular species in a sample from the individual, levels of amino acids and their constituent molecular species in a sample from the individual, levels of peptides and their constituent molecular species in a sample from the individual, or levels of nucleosides and their constituent molecular species in a sample from the individual;
wherein the clinical outcome data comprise one or more of: severity or duration of symptoms, time to symptom onset or abatement, need for organ support, duration of organ support, response to treatment, admission to a hospital or intensive care unit, length of stay in a hospital or intensive care unit, mortality, time to death, duration of morbidity (e.g., time to returning to normal daily activities or quality of life), incidence of long-term sequelae of infectious diseases, or re-hospitalization; and
wherein the administrative health data comprise one or more of: baseline demographics, physiologic parameters, comorbid conditions including but not limited to immunocompromising conditions, past surgical history, and environmental or social exposures.

10. A method for predicting severe disease in an individual with sepsis or at risk of developing sepsis comprising: receiving, from a second individual, a second value of at least one clinical parameter of a plurality of clinical parameters; executing a pre-trained model for predicting severe disease from sepsis of the second individual using the second value of at least one clinical parameter, wherein the model is pre-trained by performing operations comprising: generating a discovery database storing first values of the plurality of clinical parameters and clinical outcomes associated with a plurality of first subjects; executing a plurality of data quality control algorithms to select a subset of clinical parameters from the plurality of clinical parameters; executing topological data analysis and/or clustering for the plurality of subsets of clinical parameters; executing a plurality of feature selection machine learning and/or ensemble learning models based on a plurality of classification and/or time-to-event analysis algorithms; and outputting a model for predicting severe disease in the individual with sepsis or at risk of developing sepsis; and outputting the predicted mortality outcomes of the second individual.

11. The method of claim 10, further comprising pre-processing data that is stored in the discovery database including: determining that a first value of at least one of the plurality of clinical parameters is missing; estimating a reference value for the at least one of the plurality of clinical parameters that is missing; and storing the reference value as the first value of the at least one of the plurality of clinical parameters in the discovery database.

12. The method of claim 10 or 11, wherein the plurality of data quality control algorithms comprise at least one of: differential expression algorithms, k-nearest neighbor imputation algorithms, three-sigma rule algorithms, or empirical Bayes method algorithms.

13. The method of any one of claims 10-12, wherein the topological data analysis groups individuals or samples based on similarities in the plurality of subsets of clinical parameters, as well as the algebraic topology of the same data, wherein clusters are delineated based on the persistence homology of the node density and connectivity, and wherein sepsis response phenotypes are defined based on the identified clusters.

14. The method of any one of claims 10-12, wherein the cluster analysis discretizes the plurality of subsets of clinical parameters based on measures of similarity, and wherein sepsis response phenotypes are defined based on the identified clusters.

15. The method of any one of claims 10-14, wherein the feature selection machine learning models comprise at least one of: unsupervised machine learning algorithm, supervised machine learning algorithm, minimum redundancy maximum relevance, Student's t-test, Mann-Whitney U test, random forest, logistic regression, or neural networks.

16. The method of any one of claims 10-14, wherein the feature selection ensemble learning models comprise at least one of: cluster analysis, unsupervised machine learning algorithm, supervised machine learning algorithm, minimum redundancy maximum relevance, Student's t-test, Mann-Whitney U test, random forest, logistic regression, neural networks, Bayes optimal classifier, classification and regression tree, bootstrap aggregating, boosting, Bayesian model averaging, Bayesian model combination, a bucket of models, or stacking.

17. The method of any one of claims 10-16, wherein the plurality of clinical parameters comprise one or more nucleic acid data markers, one or more protein data markers, one or more of metabolite data markers, one or more clinical outcomes data, one or more administrative health data, or a combination thereof.

18. The method of claim 17, wherein the nucleic acid data markers comprise one or more of: level of adhesion G protein-coupled receptor E1 (ADGRE1) in a sample from the individual, level of adrenoceptor β2 (ADRB2) in a sample from the individual, level of angiotensin II receptor associated protein (AGTRAP) in a sample from the individual, level of AKT serine/threonine kinase 1 (AKT1) in a sample from the individual, level of 5′-aminolevulinate synthase 2 (ALAS2) in a sample from the individual, level of alkaline phosphatase, biomineralization associated (ALPL) in a sample from the individual, level of ankyrin repeat domain 22 (ANKRD22) in a sample from the individual, level of annexin A3 (ANXA3) in a sample from the individual, level of arginase 1 (ARG1) in a sample from the individual, level of BCL2 like 1 (BCL2L1) in a sample from the individual, level of BMX non-receptor tyrosine kinase (BMX) in a sample from the individual, level of chromosome 6 open reading frame 62 (C6orf62) in a sample from the individual, level of carbonic anhydrase 2 (CA2) in a sample from the individual, level of C-C motif chemokine ligand 5 (CCL5) in a sample from the individual, level of C-C motif chemokine receptor 3 (CCR3) in a sample from the individual, level of CD4 molecule (CD4) in a sample from the individual, level of CD24 molecule (CD24) in a sample from the individual, level of CD177 molecule (CD177) in a sample from the individual, level of CD274 molecule (CD274) in a sample from the individual, level of cell division cycle 34, ubiqiutin conjugating enzyme (CDC34) in a sample from the individual, level of complement factor D (CFD) in a sample from the individual, level of chitinase 3 like 1 (CHI3L1) in a sample from the individual, level of carbohydrate sulfotransferase 2 (CHST2) in a sample from the individual, level of C-type lectin domain family 4 member E (CLEC4E) in a sample from the individual, level of cytidine/uridine monophosphate kinase 2 (CMPK2) in a sample from the individual, level of cytochrome C oxidase assembly factor 1 homolog (COA1) in a sample from the individual, level of carnitine palmitoyltransferase 1A (CPT1A) in a sample from the individual, level of carboxypeptidase vitellogenic like (CPVL) in a sample from the individual, level of chondroitin sulfate N-acetylgalactosaminyltransferase 1 (CSGALNACT1) in a sample from the individual, level of cystatin C (CST3) in a sample from the individual, level of C-X3-C motif chemokine receptor 1 (CX3CR1) in a sample from the individual, level of DNA damage inducible transcript 4 (DDIT4) in a sample from the individual, level of defensin α3 (DEFA3) in a sample from the individual, level of defensin α4 (DEFA4) in a sample from the individual, level of DNA J heat shock protein family (Hsp40) member C1 (DNAJC1) in a sample from the individual, level of DNA damage regulated autophagy modulator 1 (DRAM1) in a sample from the individual, level of deoxyuridine triphosphatase (DUT) in a sample from the individual, level of dual specificity tyrosine phosphorylation regulated kinase 3 (DYRK3) in a sample from the individual, level of erythrocyte membrane protein band 4.2 (EPB42) in a sample from the individual, level of family with sequence similarity 174 member C (FAM174C) in a sample from the individual, level of F-box and WD repeat domain containing 2 (FBXW2) in a sample from the individual, level of Fc receptor like 5 (FCRL5) in a sample from the individual, level of ferrochelatase (FECH) in a sample from the individual, level of fibroblast growth factor binding protein 2 (FGFBP2) in a sample from the individual, level of Fms related receptor tyrosine kinase 3 (FLT3) in a sample from the individual, level of formyl peptide receptor 1 (FPR1) in a sample from the individual, level of GATA binding protein 1 (GATA1) in a sample from the individual, level of GTPase, IMAP family member 4 (GIMAP4) in a sample from the individual, level of GTPase, IMAP family member 7 (GIMAP7) in a sample from the individual, level of GTPase, IMAP family member 8 (GIMAP8) in a sample from the individual, level of G protein subunit γ2 (GNG2) in a sample from the individual, level of granulysin (GNLY) in a sample from the individual, level of G protein-coupled receptor 65 (GPR65) in a sample from the individual, level of growth factor receptor bound protein 10 (GRB10) in a sample from the individual, level of glutathione S-transferase κ1 (GSTK1) in a sample from the individual, level of H3 histone pseudogene 6 (H3F3AP4) in a sample from the individual, level of hemoglobin subunit α2 (HBA2) in a sample from the individual, level of hemogen (HEMGN) in a sample from the individual, level of HECT and RLD domain containing E3 ubiquitin protein ligase family member 6 (HERC6) in a sample from the individual, level of H3.2 histone [putative] (HIST2H3PS2) in a sample from the individual, level of major histocompatibility complex, class I, B (HLA-B) in a sample from the individual, level of major histocompatibility complex, class II, DQ β1 (HLA-DQB1) in a sample from the individual, level of high mobility group box 2 (HMGB2) in a sample from the individual, level of 15-hydroxyprostaglandin dehydrogenase (HPGD) in a sample from the individual, level of hydrogen voltage gated channel 1 (HVCN1) in a sample from the individual, level of isoamyl acetate hydrolyzing esterase 1 [putative] (IAH1) in a sample from the individual, level of intercellular adhesion molecule 1 (ICAM1) in a sample from the individual, level of immediate early response 5 (IER5) in a sample from the individual, level of interferon α inducible protein 6 (IFI6) in a sample from the individual, level of interferon α inducible protein 27 (IFI27) in a sample from the individual, level of interferon induced protein 44 (IFI44) in a sample from the individual, level of interferon induced protein with tetratricopeptide repeats 1 (IFIT1) in a sample from the individual, level of interferon induced protein with tetratricopeptide repeats 2 (IFIT2) in a sample from the individual, level of interleukin 1β (IL1B) in a sample from the individual, level of interleukin 1 receptor type 1 (IL1RA) in a sample from the individual, level of interleukin 1 receptor type 2 (IL1R2) in a sample from the individual, level of interleukin 10 receptor subunit α (IL10RA) in a sample from the individual, level of interaction protein for cytohesin exchange factors 1 (IPCEF1) in a sample from the individual, level of interferon regulatory factor 2 binding protein 2 (IRF2BP2) in a sample from the individual, level of ISG15 ubiquitin like modifier (ISG15) in a sample from the individual, level of JUN proto-oncogene, AP-1 transcription factor subunit (JUN) in a sample from the individual, level of potassium voltage-gated channel subfamily E regulatory subunit 1 (KCNE1) in a sample from the individual, level of kinesin light chain 3 (KLC3) in a sample from the individual, level of kelch like family member 24 (KLHL24) in a sample from the individual, level of kringle containing transmembrane protein 1 (KREMEN1) in a sample from the individual, level of long intergenic non-protein coding RNA 861 (LINC00861) in a sample from the individual, level of lymphocyte antigen 6 family member E (LY6E) in a sample from the individual, level of MAPK associated protein 1 (MAPKAP1) in a sample from the individual, level of mediator complex subunit 28 (MED28) in a sample from the individual, level of MicroRNA 6724-4 (MIR6724-4) in a sample from the individual, level of matrix metallopeptidase 8 (MMP8) in a sample from the individual, level of multimerin 1 (MMRN1) in a sample from the individual, level of myeloperoxidase (MPO) in a sample from the individual, level of mannose receptor C type 2 (MRC2) in a sample from the individual, level of mitochondrially encoded 12S rRNA (MT-RNR1) in a sample from the individual, level of MX dynamin like GTPase 2 (MX2) in a sample from the individual, level of nuclear factor, erythroid 2 like 3 (NFE2L3) in a sample from the individual, level of 2′-5′-oligoadenylate synthetase 3 (OAS3) in a sample from the individual, level of oleoyl-ACP hydrolase (OLAH) in a sample from the individual, level of olfactomedin 4 (OLFM4) in a sample from the individual, level of peptidase inhibitor 3 (PI3) in a sample from the individual, level of phosphatidylinositol-4,5-bisphosphate 3-kinase catalytic subunit β (PIK3CB) in a sample from the individual, level of PITH domain containing 1 (PITHD1) in a sample from the individual, level of pyruvate kinase M1/2 (PKM) in a sample from the individual, level of perilipin 2 (PLIN2) in a sample from the individual, level of DNA polymerase δ interacting protein 3 (POLDIP3) in a sample from the individual, level of RAL GTPase activating protein catalytic subunit α2 (RALGAPA2) in a sample from the individual, level of RAN binding protein 9 (RANBP9) in a sample from the individual, level of REST corepressor 1 (RCOR1) in a sample from the individual, level of Rh associated glycoprotein (RHAG) in a sample from the individual, level of RNA, U1 small nuclear 2 (RNU1-2) in a sample from the individual, level of RNA, U1 small nuclear 4 (RNU1-4) in a sample from the individual, level of ribosomal protein L37a (RPL37A) in a sample from the individual, level of ribosomal protein L38 (RPL38) in a sample from the individual, level of ribosomal protein S11 (RPS11) in a sample from the individual, level of ribosomal protein S18 (RPS18) in a sample from the individual, level of radical S-adenosyl methionine domain containing 2 (RSAD2) in a sample from the individual, level of S100 calcium binding protein A8 (S100A8) in a sample from the individual, level of S100 calcium binding protein A9 (S100A9) in a sample from the individual, level of S100 calcium binding protein A12 (S100A12) in a sample from the individual, level of SAM domain, SH3 domain and nuclear localization signals 1 (SAMSN1) in a sample from the individual, level of Sin3A associated protein 30 (SAP30) in a sample from the individual, level of strawberry notch homolog 1 (SBNO1) in a sample from the individual, level of selenium binding protein 1 (SELENBP1) in a sample from the individual, level of sialic acid binding Ig like lectin 10 (SIGLEC10) in a sample from the individual, level of solute carrier family 25 member 6 (SLC25A6) in a sample from the individual, level of solute carrier family 25 member 39 (SLC25A39) in a sample from the individual, level of solute carrier family 39 member 8 (SLC39A8) in a sample from the individual, level of solute carrier family 4 member 1 [Diego Blood Group] (SLC4A1) in a sample from the individual, level of synuclein α (SNCA) in a sample from the individual, level of small nucleolar RNA, H/ACA box 44 (SNORA44) in a sample from the individual, level of superoxide dismutase 2 (SOD2) in a sample from the individual, level of spectrin α, erythrocytic 1 (SPTA1) in a sample from the individual, level of STE20 related adaptor β (STRADB) in a sample from the individual, level of syntaxin 6 (STX6) in a sample from the individual, level of switching B cell complex subunit SWAP70 (SWAP70) in a sample from the individual, level of spectrin repeat containing nuclear envelope protein 2 (SYNE2) in a sample from the individual, level of T-box transcription factor 21 (TBX21) in a sample from the individual, level of TRAF interacting protein with forkhead associated domain (TIFA) in a sample from the individual, level of toll like receptor 7 (TLR7) in a sample from the individual, level of transmembrane and coiled-coil domain family 2 (TMCC2) in a sample from the individual, level of transmembrane protein 35B (TMEM35B) in a sample from the individual, level of transmembrane protein 273 (TMEM273) in a sample from the individual, level of thymosin β10 (TMSB10) in a sample from the individual, level of TNF α induced protein 6 (TNFAIP6) in a sample from the individual, level of tyrosylprotein sulfotransferase 1 (TPST1) in a sample from the individual, level of tripartite motif containing 4 (TRIM4) in a sample from the individual, level of tetraspanin 5 (TSPAN5) in a sample from the individual, level of tetratricopeptide repeat domain 9C (TTC9C) in a sample from the individual, level of ubiquitin protein ligase E3 component N-recognin 5 (UBR5) in a sample from the individual, level of UNC-93 homolog B1, TLR signaling regulator (UNC93B1) in a sample from the individual, level of WASH complex subunit 2C (WASHC2C) in a sample from the individual, level of XIAP associated factor 1 (XAF1) in a sample from the individual, level of tyrosine 3-monooxygenase/tryptophan 5-monooxygenase activation protein E (YWHAH) in a sample from the individual, or level of or zinc finger With KRAB and SCAN domains 1 (ZKSCAN1) in a sample from the individual;
wherein the protein data markers comprise one or more of: level of a disintegrin and metalloproteinase with thrombospondin motifs 13 (ADAMTS13) in a sample from the individual, level of Angiopoietin-1 (ANGPT1) in a sample from the individual, level of Angiopoietin-2 (ANGPT2) in a sample from the individual, level of C-C chemokine receptor ligand 2/monocyte chemoattractant protein 1 (CCL2/MCP-1) in a sample from the individual, level of C-C chemokine receptor ligand 3/macrophage inflammatory protein 1-alpha (CCL3/MIP-1-α) in a sample from the individual, level of C-C chemokine receptor ligand 5/regulated on activation, normal T cell expressed and secreted (CCL5/RANTES) in a sample from the individual, level of cluster of differentiation 163 (CD163) in a sample from the individual, level of cluster of differentiation 40 ligand (CD40L) in a sample from the individual, level of chitinase-3-like protein 1 (CHI3L1) in a sample from the individual, level of C-reactive protein (CRP) in a sample from the individual, level of C-X-C motif chemokine ligand 10/interferon gamma-induced protein 10 (CXCL10/IP-10) in a sample from the individual, level of decoy receptor 3 (Dcr3) in a sample from the individual, level of D-dimer in a sample from the individual, level of E-selectin (SELE) in a sample from the individual, level of endoglin (ENG) in a sample from the individual, level of fas receptor (FAS) in a sample from the individual, level of ferritins in a sample from the individual, level of fibrinogens in a sample from the individual, level of granulocyte colony-stimulating factor (G-CSF) in a sample from the individual, level of granulocyte-macrophage colony-stimulating factor (GM-CSF) in a sample from the individual, level of (soluble) intercellular adhesion molecule 1 (ICAM-1) in a sample from the individual, level of interferon gamma (IFNγ) in a sample from the individual, level of interleukin 1 beta (IL-1β) in a sample from the individual, level of interleukin-1 receptor antagonist (IL-1RA) in a sample from the individual, level of (soluble) interleukin-2 receptor α (IL-2Rα) in a sample from the individual, level of interleukin-4 (IL-4) in a sample from the individual, level of interleukin-5 (IL-5) in a sample from the individual, level of interleukin-6 (IL-6) in a sample from the individual, level of interleukin-6 receptor α (IL-6Rα) in a sample from the individual, level of interleukin-7 (IL-7) in a sample from the individual, level of interleukin-8 (IL-8) in a sample from the individual, level of interleukin-10 (IL-10) in a sample from the individual, level of interleukin-12 ‘p70’ (IL-12 p70) in a sample from the individual, level of interleukin-15 (IL-15) in a sample from the individual, level of interleukin-16 (IL-16) in a sample from the individual, level of interleukin-17A (IL-17A) in a sample from the individual, level of interleukin-18 (IL-18) in a sample from the individual, level of interleukin-18-binding protein (IL-18BP) in a sample from the individual, level of interleukin-22 (IL-22) in a sample from the individual, level of interleukin-27 (IL-27) in a sample from the individual, level of lipocalin-2 (LCN-2) in a sample from the individual, level of matrix metalloproteinase-8 (MMP-8) in a sample from the individual, level of matrix metalloproteinase-9 (MMP-9) in a sample from the individual, level of matrix metalloproteinase-10 (MMP-10) in a sample from the individual, level of (soluble) macrophage mannose receptors in a sample from the individual, level of procalcitonin (PCT) in a sample from the individual, level of (soluble) programmed death-ligand 1 (PD-L1) in a sample from the individual, level of pentraxin 3 (PTX3) in a sample from the individual, level of (soluble) receptor for advanced glycation end products (RAGE) in a sample from the individual, level of resistin (RETN) in a sample from the individual, level of serum amyloid A proteins (SAA) in a sample from the individual, level of tyrosine kinase with immunoglobulin-like and EGF-like domains 1 (TIE1) in a sample from the individual, level of tyrosine kinase with immunoglobulin-like and EGF-like domains 2 (TIE2) in a sample from the individual, level of tissue inhibitor of metalloproteinases 1 (TIMP1) in a sample from the individual, level of tissue inhibitor of metalloproteinases 2 (TIMP2) in a sample from the individual, level of tissue inhibitor of metalloproteinases 3 (TIMP3) in a sample from the individual, level of tissue inhibitor of metalloproteinases 4 (TIMP4) in a sample from the individual, level of tumor necrosis factor receptor 1 (TNF-R1) in a sample from the individual, level of tumor necrosis factor alpha (TNFα) in a sample from the individual, level of tissue plasminogen activator (tPA) in a sample from the individual, level of tissue plasminogen activator inhibitor 1 (tPAI-1) in a sample from the individual, level of TNF-related apoptosis-inducing ligand (TRAIL) in a sample from the individual, level of (soluble) triggering receptor expressed on myeloid cells 1 (TREM1) in a sample from the individual, level of urokinase receptor (uPar) in a sample from the individual, level of (soluble) vascular cell adhesion molecule 1 (VCAM-1) in a sample from the individual, level of vascular endothelial growth factors (VEGF) in a sample from the individual, level of (soluble) vascular endothelial growth factor receptor 1 (VEGFR-1) in a sample from the individual, level of (soluble) vascular endothelial growth factor receptor 2 (VEGFR-2) in a sample from the individual, or level of von Willebrand factor A2 domain (vWF-A2) in a sample from the individual;
wherein the metabolite data comprise one or more of: levels of fatty acyls and their constituent molecular species in a sample from the individual, levels of glycerolipids and their constituent molecular species in a sample from the individual, levels of glycerophospholipids and their constituent molecular species in a sample from the individual, levels of sphingolipids and their constituent molecular species in a sample from the individual, levels of sterol lipids and their constituent molecular species in a sample from the individual, levels of prenol lipids and their constituent molecular species in a sample from the individual, levels of saccharolipids and their constituent molecular species in a sample from the individual, levels of polyketides and their constituent molecular species in a sample from the individual, levels of carbohydrates and their constituent molecular species in a sample from the individual, levels of organic acids and their derivatives and constituent molecular species in a sample from the individual, levels of organo-heterocyclic compounds and their constituent molecular species in a sample from the individual, levels of organo-oxygen compounds and their constituent molecular species in a sample from the individual, levels of organo-nitrogen compounds and their constituent molecular species in a sample from the individual, levels of amino acids and their constituent molecular species in a sample from the individual, levels of peptides and their constituent molecular species in a sample from the individual, or levels of nucleosides and their constituent molecular species in a sample from the individual;
wherein the clinical outcome data comprise one or more of: severity or duration of symptoms, time to symptom onset or abatement, need for organ support, duration of organ support, response to treatment, admission to a hospital or intensive care unit, length of stay in a hospital or intensive care unit, mortality, time to death, duration of morbidity (e.g., time to returning to normal daily activities or quality of life), incidence of long-term sequelae of infectious diseases, or re-hospitalization; and
wherein the administrative health data comprise one or more of: baseline demographics, physiologic parameters, comorbid conditions including but not limited to immunocompromising conditions, past surgical history, and environmental or social exposures.

19. The method of any one of claims 10-18, wherein the method further comprises treating the individual or adjusting current treatment for the individual to prevent or ameliorate severe disease from sepsis based on the model.

20. The method of claim 19, wherein treating the individual comprises at least one of initiation or broadening of antibiotic therapy, balancing fluids and electrolytes, renal replacement therapy, adjustment of mechanical ventilation, targeted or empiric anti-inflammatory or immunomodulatory drugs, hemodynamic adjustments, calcium channel blocker medications, or surgical intervention.

21. The method of claim 19, wherein adjusting current treatment comprises changing dose of current antibiotic, changing to a different antibiotic, changing dose of non-steroidal anti-inflammatory drugs, initiating or adjusting insulin therapy.

22. A system for generating a machine learning engine for predicting severe disease in an individual with sepsis or at risk of developing sepsis, comprising: one or more processors; a memory; a communication platform; a discovery database configured to store first values of a plurality of clinical parameters and clinical outcomes associated with a plurality of first subjects; and a machine learning engine configured to: execute a plurality of data quality control algorithms to select a subset of clinical parameters from the plurality of clinical parameters; execute topological data analysis and/or clustering for the plurality of subsets of clinical parameters; execute a plurality of feature selection machine learning and/or ensemble learning models based on a plurality of classification and/or time-to-event analysis algorithms; and output a model for predicting severe disease in the individual with sepsis or at risk of developing sepsis.

23. The system of claim 22, further comprising configuring the model to predict severe disease in the individual with sepsis or at risk of developing sepsis and instantiating the model in a prediction engine that is accessed by a remote device connected to the system via a network.

24. The system of claim 22 or 23, wherein the communication platform comprises at least one of: a mobile device, a secured network, a server that stores and receives messages, and a database.

25. A system for predicting severe disease in an individual with sepsis or at risk of developing sepsis, comprising: one or more processors; a memory; a communication platform; a discovery database configured to store first values of a plurality of clinical parameters and the clinical outcomes associated with a plurality of first subjects; a machine learning engine configured to pre-train a model for severe disease in an individual with sepsis or at risk of developing sepsis, wherein the model is pre-trained by performing operations comprising: executing a plurality of data quality control algorithms to select a subset of clinical parameters from the plurality of clinical parameters; executing topological data analysis and/or clustering for the plurality of subsets of clinical parameters; executing a plurality of feature selection machine learning and/or ensemble learning models based on a plurality of classification and/or time-to-event analysis algorithms; outputting a model for predicting severe disease in the individual with sepsis or at risk of developing sepsis; a prediction engine configured to: receive, from a second individual, a second value of at least one clinical parameter of a plurality of clinical parameters; and execute the pre-trained model for predicting severe disease of the second individual using the second value of at least one clinical parameter; and a display device configured to output the predicted severe disease of the second individual.

26. A non-transitory computer-readable medium having information recorded thereon for generating a model for predicting severe disease in an individual with sepsis or at risk of developing sepsis, wherein the information, when read by a computer, causes the computer to perform operations of: generating a discovery database storing first values of a plurality of clinical parameters and clinical outcomes associated with a plurality of first subjects; executing a plurality of data quality control algorithms to select a subset of clinical parameters from the plurality of clinical parameters; executing topological data analysis and/or clustering for the plurality of subsets of clinical parameters; executing a plurality of feature selection machine learning and/or ensemble learning models based on a plurality of classification and/or time-to-event analysis algorithms; and outputting a model for predicting severe disease in the individual with sepsis or at risk of developing sepsis.

27. An array of host-biomarkers for sepsis, wherein the array of biomarkers comprise two or more of: adhesion G protein-coupled receptor E1 (ADGRE1), adrenoceptor β2 (ADRB2), angiotensin II receptor associated protein (AGTRAP), AKT serine/threonine kinase 1 (AKT1), 5′-aminolevulinate synthase 2 (ALAS2), alkaline phosphatase, biomineralization associated (ALPL), ankyrin repeat domain 22 (ANKRD22), annexin A3 (ANXA3), arginase 1 (ARG1), BCL2 like 1 (BCL2L1), BMX non-receptor tyrosine kinase (BMX), chromosome 6 open reading frame 62 (C6orf62), carbonic anhydrase 2 (CA2), C-C motif chemokine ligand 5 (CCL5), C-C motif chemokine receptor 3 (CCR3), CD4 molecule (CD4), CD24 molecule (CD24), CD177 molecule (CD177), CD274 molecule (CD274), cell division cycle 34, ubiqiutin conjugating enzyme (CDC34), complement factor D (CFD), chitinase 3 like 1 (CHI3L1), carbohydrate sulfotransferase 2 (CHST2), C-type lectin domain family 4 member E (CLEC4E), cytidine/uridine monophosphate kinase 2 (CMPK2), cytochrome C oxidase assembly factor 1 homolog (COA1), carnitine palmitoyltransferase 1A (CPT1A), carboxypeptidase vitellogenic like (CPVL), chondroitin sulfate N-acetylgalactosaminyltransferase 1 (CSGALNACT1), cystatin C (CST3), C-X3-C motif chemokine receptor 1 (CX3CR1), DNA damage inducible transcript 4 (DDIT4), defensin α3 (DEFA3), defensin α4 (DEFA4), DNA J heat shock protein family (Hsp40) member C1 (DNAJC1), DNA damage regulated autophagy modulator 1 (DRAM1), deoxyuridine triphosphatase (DUT), dual specificity tyrosine phosphorylation regulated kinase 3 (DYRK3), erythrocyte membrane protein band 4.2 (EPB42), family with sequence similarity 174 member C (FAM174C), F-box and WD repeat domain containing 2 (FBXW2), Fc receptor like 5 (FCRL5), ferrochelatase (FECH), fibroblast growth factor binding protein 2 (FGFBP2), Fms related receptor tyrosine kinase 3 (FLT3), formyl peptide receptor 1 (FPR1), GATA binding protein 1 (GATA1), GTPase, IMAP family member 4 (GIMAP4), GTPase, IMAP family member 7 (GIMAP7), GTPase, IMAP family member 8 (GIMAP8), G protein subunit γ2 (GNG2), granulysin (GNLY), G protein-coupled receptor 65 (GPR65), growth factor receptor bound protein 10 (GRB10), glutathione S-transferase κ1 (GSTK1), H3 histone pseudogene 6 (H3F3AP4), hemoglobin subunit α2 (HBA2), hemogen (HEMGN), HECT and RLD domain containing E3 ubiquitin protein ligase family member 6 (HERC6), H3.2 histone [putative] (HIST2H3PS2), major histocompatibility complex, class I, B (HLA-B), major histocompatibility complex, class II, DQ β1 (HLA-DQB1), high mobility group box 2 (HMGB2), 15-hydroxyprostaglandin dehydrogenase (HPGD), hydrogen voltage gated channel 1 (HVCN1), isoamyl acetate hydrolyzing esterase 1 [putative] (IAH1), intercellular adhesion molecule 1 (ICAM1), immediate early response 5 (IER5), interferon α inducible protein 6 (IFI6), interferon α inducible protein 27 (IFI27), interferon induced protein 44 (IFI44), interferon induced protein with tetratricopeptide repeats 1 (IFIT1), interferon induced protein with tetratricopeptide repeats 2 (IFIT2), interleukin 1β (IL1B), interleukin 1 receptor type 1 (IL1RA), interleukin 1 receptor type 2 (IL1R2), interleukin 10 receptor subunit α (IL10RA), interaction protein for cytohesin exchange factors 1 (IPCEF1), interferon regulatory factor 2 binding protein 2 (IRF2BP2), ISG15 ubiquitin like modifier (ISG15), JUN proto-oncogene, AP-1 transcription factor subunit (JUN), potassium voltage-gated channel subfamily E regulatory subunit 1 (KCNE1), kinesin light chain 3 (KLC3), kelch like family member 24 (KLHL24), kringle containing transmembrane protein 1 (KREMEN1), long intergenic non-protein coding RNA 861 (LINC00861), lymphocyte antigen 6 family member E (LY6E), MAPK associated protein 1 (MAPKAP1), mediator complex subunit 28 (MED28), MicroRNA 6724-4 (MIR6724-4), matrix metallopeptidase 8 (MMP8), multimerin 1 (MMRN1), myeloperoxidase (MPO), mannose receptor C type 2 (MRC2), mitochondrially encoded 12S rRNA (MT-RNR1), MX dynamin like GTPase 2 (MX2), nuclear factor, erythroid 2 like 3 (NFE2L3), 2′-5′-oligoadenylate synthetase 3 (OAS3), oleoyl-ACP hydrolase (OLAH), olfactomedin 4 (OLFM4), peptidase inhibitor 3 (PI3), phosphatidylinositol-4,5-bisphosphate 3-kinase catalytic subunit β (PIK3CB), PITH domain containing 1 (PITHD1), pyruvate kinase M1/2 (PKM), perilipin 2 (PLIN2), DNA polymerase δ interacting protein 3 (POLDIP3), RAL GTPase activating protein catalytic subunit α2 (RALGAPA2), RAN binding protein 9 (RANBP9), REST corepressor 1 (RCOR1), Rh associated glycoprotein (RHAG), RNA, U1 small nuclear 2 (RNU1-2), RNA, U1 small nuclear 4 (RNU1-4), ribosomal protein L37a (RPL37A), ribosomal protein L38 (RPL38), ribosomal protein S11 (RPS11), ribosomal protein S18 (RPS18), radical S-adenosyl methionine domain containing 2 (RSAD2), S100 calcium binding protein A8 (S100A8), S100 calcium binding protein A9 (S100A9), S100 calcium binding protein A12 (S100A12), SAM domain, SH3 domain and nuclear localization signals 1 (SAMSN1), Sin3A associated protein 30 (SAP30), strawberry notch homolog 1 (SBNO1), selenium binding protein 1 (SELENBP1), sialic acid binding Ig like lectin 10 (SIGLEC10), solute carrier family 25 member 6 (SLC25A6), solute carrier family 25 member 39 (SLC25A39), solute carrier family 39 member 8 (SLC39A8), solute carrier family 4 member 1 [Diego Blood Group] (SLC4A1), synuclein α (SNCA), small nucleolar RNA, H/ACA box 44 (SNORA44), superoxide dismutase 2 (SOD2), spectrin α, erythrocytic 1 (SPTA1), STE20 related adaptor β (STRADB), syntaxin 6 (STX6), switching B cell complex subunit SWAP70 (SWAP70), spectrin repeat containing nuclear envelope protein 2 (SYNE2), T-box transcription factor 21 (TBX21), TRAF interacting protein with forkhead associated domain (TIFA), toll like receptor 7 (TLR7), transmembrane and coiled-coil domain family 2 (TMCC2), transmembrane protein 35B (TMEM35B), transmembrane protein 273 (TMEM273), thymosin β10 (TMSB10), TNF α induced protein 6 (TNFAIP6), tyrosylprotein sulfotransferase 1 (TPST1), tripartite motif containing 4 (TRIM4), tetraspanin 5 (TSPAN5), tetratricopeptide repeat domain 9C (TTC9C), ubiquitin protein ligase E3 component N-recognin 5 (UBR5), UNC-93 homolog B1, TLR signaling regulator (UNC93B1), WASH complex subunit 2C (WASHC2C), XIAP associated factor 1 (XAF1), tyrosine 3-monooxygenase/tryptophan 5-monooxygenase activation protein E (YWHAH), and zinc finger With KRAB and SCAN domains 1 (ZKSCAN1), a disintegrin and metalloproteinase with thrombospondin motifs 13 (ADAMTS13), Angiopoietin-1 (ANGPT1), Angiopoietin-2 (ANGPT2), C-C chemokine receptor ligand 2/monocyte chemoattractant protein 1 (CCL2/MCP-1), C-C chemokine receptor ligand 3/macrophage inflammatory protein 1-alpha (CCL3/MIP-1-α), C-C chemokine receptor ligand 5/regulated on activation, normal T cell expressed and secreted (CCL5/RANTES), cluster of differentiation 163 (CD163), cluster of differentiation 40 ligand (CD40L), chitinase-3-like protein 1 (CHI3L1), C-reactive protein (CRP), C-X-C motif chemokine ligand 10/interferon gamma-induced protein 10 (CXCL10/IP-10), decoy receptor 3 (Dcr3), D-dimer, E-selectin (SELE), endoglin (ENG), fas receptor (FAS), ferritins, fibrinogens, granulocyte colony-stimulating factor (G-CSF), granulocyte-macrophage colony-stimulating factor (GM-CSF), (soluble) intercellular adhesion molecule 1 (ICAM-1), interferon gamma (IFNγ), interleukin 1 beta (IL-1β), interleukin-1 receptor antagonist (IL-1RA), (soluble) interleukin-2 receptor α (IL-2Rα), interleukin-4 (IL-4), interleukin-5 (IL-5), interleukin-6 (IL-6), interleukin-6 receptor α (IL-6Rα), interleukin-7 (IL-7), interleukin-8 (IL-8), interleukin-10 (IL-10), interleukin-12 ‘p70’ (IL-12 p70), interleukin-15 (IL-15), interleukin-16 (IL-16), interleukin-17A (IL-17A), interleukin-18 (IL-18), interleukin-18-binding protein (IL-18BP), interleukin-22 (IL-22), interleukin-27 (IL-27), lipocalin-2 (LCN-2), matrix metalloproteinase-8 (MMP-8), matrix metalloproteinase-9 (MMP-9), matrix metalloproteinase-10 (MMP-10), (soluble) macrophage mannose receptors, procalcitonin (PCT), (soluble) programmed death-ligand 1 (PD-L1), pentraxin 3 (PTX3), (soluble) receptor for advanced glycation end products (RAGE), resistin (RETN), serum amyloid A proteins (SAA), tyrosine kinase with immunoglobulin-like and EGF-like domains 1 (TIE1), tyrosine kinase with immunoglobulin-like and EGF-like domains 2 (TIE2), tissue inhibitor of metalloproteinases 1 (TIMP1), tissue inhibitor of metalloproteinases 2 (TIMP2), tissue inhibitor of metalloproteinases 3 (TIMP3), tissue inhibitor of metalloproteinases 4 (TIMP4), tumor necrosis factor receptor 1 (TNF-R1), tumor necrosis factor alpha (TNFα), tissue plasminogen activator (tPA), tissue plasminogen activator inhibitor 1 (tPAI-1), TNF-related apoptosis-inducing ligand (TRAIL), (soluble) triggering receptor expressed on myeloid cells 1 (TREM1), urokinase receptor (uPar), (soluble) vascular cell adhesion molecule 1 (VCAM-1), vascular endothelial growth factors (VEGF), (soluble) vascular endothelial growth factor receptor 1 (VEGFR-1), (soluble) vascular endothelial growth factor receptor 2 (VEGFR-2), von Willebrand factor A2 domain (vWF-A2), fatty acyls and their constituent molecular species, glycerolipids and their constituent molecular species, glycerophospholipids and their constituent molecular species, sphingolipids and their constituent molecular species, sterol lipids and their constituent molecular species, prenol lipids and their constituent molecular species, saccharolipids and their constituent molecular species, polyketides and their constituent molecular species, carbohydrates and their constituent molecular species, organic acids and their derivatives and constituent molecular species, organo-heterocyclic compounds and their constituent molecular species, organo-oxygen compounds and their constituent molecular species, organo-nitrogen compounds and their constituent molecular species, amino acids and their constituent molecular species, peptides and their constituent molecular species, or nucleosides and their constituent molecular species.

28. The array of biomarkers of claim 27, wherein the array is an array of nucleic acids, an array of peptides, or an array of metabolites.

29. The array of biomarkers of claim 27 or 28, wherein the array comprises three or more biomarkers, four or more biomarkers, five or more biomarkers, six or more biomarkers, seven or more biomarkers, eight of more biomarkers, nine or more biomarkers, 10 or more biomarkers, 15 or more biomarkers, 20 or more biomarkers, 25 or more biomarkers, 30 or more biomarkers, 35 or more biomarkers, 40 or more biomarkers, 45 or more biomarkers, or 48 biomarkers
30. A method of predicting mortality in an individual with sepsis comprising: obtaining a biological sample from the individual; measuring one or more of the following biomarkers: adrenoceptor β2 (ADRB2), CD177 molecule (CD177), carboxypeptidase vitellogenic like (CPVL), C-X3-C motif chemokine receptor 1 (CX3CR1), defensin α3 (DEFA3), Fc receptor like 5 (FCRL5), G protein subunit γ2 (GNG2), interleukin 10 receptor subunit α (IL10RA), kinesin light chain 3 (KLC3), oleoyl-ACP hydrolase (OLAH), pyruvate kinase M1/2 (PKM), radical S-adenosyl methionine domain containing 2 (RSAD2), STE20 related adaptor β (STRADB), tyrosylprotein sulfotransferase 1 (TPST1), tetraspanin 5 (TSPAN5), tetratricopeptide repeat domain 9C (TTC9C), zinc finger with KRAB and SCAN domains 1 (ZKSCAN1), C-reactive protein (CRP), C-X-C motif chemokine ligand 10/interferon gamma-induced protein 10 (CXCL10/IP-10), D-dimer, ferritins, fibrinogens, (soluble) intercellular adhesion molecule 1 (ICAM-1), interferon gamma (IFNγ), interleukin-1 receptor antagonist (IL-1RA), interleukin-5 (IL-5), interleukin-6 (IL-6), interleukin-6 receptor α (IL-6Rα), interleukin-18 (IL-18), interleukin-18-binding protein (IL-18BP), lipocalin-2 (LCN-2), matrix metalloproteinase-8 (MMP-8), procalcitonin (PCT), (soluble) receptor for advanced glycation end products (RAGE), tumor necrosis factor receptor 1 (TNF-R1), tumor necrosis factor alpha (TNFα), vascular endothelial growth factors (VEGF), von Willebrand factor A2 domain (vWF-A2), carnitine, acetylcarnitine, propionylcarnitine, malonylcarnitine, methylmalonylcarnitine, hydroxypropionylcarnitine, propenoylcarnitine, butyrylcarnitine, hydroxybutyrylcarnitine, fumarylcarnitine, valerylcarnitine, glutarylcarnitine, hydroxyvalerylcarnitine, tiglylcarnitine, hexanoylcarnitine, hydroxyhexanoylcarnitine, pimeloylcarnitine, decanoylcarnitine, decadienylcarnitine, tetradecenoylcarnitine, hydroxytetradecenoylcarnitine, hydroxytetradecadienylcarnitine, hexadecanoylcarnitine, hexadecenoylcarnitine, octadecanoylcarnitine, octadecenoylcarnitine, lysophosphatidylcholine with acyl residue sum C16:0, lysophosphatidylcholine with acyl residue sum C16:1, lysophosphatidylcholine with acyl residue sum C17:0, lysophosphatidylcholine with acyl residue sum C18:0, lysophosphatidylcholine with acyl residue sum C18:1, lysophosphatidylcholine with acyl residue sum C18:2, lysophosphatidylcholine with acyl residue sum C20:3, lysophosphatidylcholine with acyl residue sum C20:4, lysophosphatidylcholine with acyl residue sum C24:0, lysophosphatidylcholine with acyl residue sum C26:0, lysophosphatidylcholine with acyl residue sum C26:1, lysophosphatidylcholine with acyl residue sum C28:0, lysophosphatidylcholine with acyl residue sum C28:1, phosphatidylcholine with diacyl residue sum C24:0, phosphatidylcholine with diacyl residue sum C28:1, phosphatidylcholine with diacyl residue sum C30:0, phosphatidylcholine with diacyl residue sum C32:0, phosphatidylcholine with diacyl residue sum C32:1, phosphatidylcholine with diacyl residue sum C32:3, phosphatidylcholine with diacyl residue sum C34:1, phosphatidylcholine with diacyl residue sum C34:2, phosphatidylcholine with diacyl residue sum C34:3, phosphatidylcholine with diacyl residue sum C34:4, phosphatidylcholine with diacyl residue sum C36:0, phosphatidylcholine with diacyl residue sum C36:1, phosphatidylcholine with diacyl residue sum C36:2, phosphatidylcholine with diacyl residue sum C36:3, phosphatidylcholine with diacyl residue sum C36:4, phosphatidylcholine with diacyl residue sum C36:5, phosphatidylcholine with diacyl residue sum C36:6, phosphatidylcholine with diacyl residue sum C38:0, phosphatidylcholine with diacyl residue sum C38:3, phosphatidylcholine with diacyl residue sum C38:4, phosphatidylcholine with diacyl residue sum C38:5, phosphatidylcholine with diacyl residue sum C38:6, phosphatidylcholine with diacyl residue sum C40:2, phosphatidylcholine with diacyl residue sum C40:3, phosphatidylcholine with diacyl residue sum C40:4, phosphatidylcholine with diacyl residue sum C40:5, phosphatidylcholine with diacyl residue sum C40:6, phosphatidylcholine with diacyl residue sum C42:0, phosphatidylcholine with diacyl residue sum C42:1, phosphatidylcholine with diacyl residue sum C42:2, phosphatidylcholine with diacyl residue sum C42:4, phosphatidylcholine with diacyl residue sum C42:5, phosphatidylcholine with diacyl residue sum C42:6, phosphatidylcholine with acyl-alkyl residue sum C30:0, phosphatidylcholine with acyl-alkyl residue sum C30:1, phosphatidylcholine with acyl-alkyl residue sum C30:2, phosphatidylcholine with acyl-alkyl residue sum C32:1, phosphatidylcholine with acyl-alkyl residue sum C32:2, phosphatidylcholine with acyl-alkyl residue sum C34:0, phosphatidylcholine with acyl-alkyl residue sum C34:1, phosphatidylcholine with acyl-alkyl residue sum C34:2, phosphatidylcholine with acyl-alkyl residue sum C34:3, phosphatidylcholine with acyl-alkyl residue sum C36:0, phosphatidylcholine with acyl-alkyl residue sum C36:1, phosphatidylcholine with acyl-alkyl residue sum C36:2, phosphatidylcholine with acyl-alkyl residue sum C36:3, phosphatidylcholine with acyl-alkyl residue sum C36:4, phosphatidylcholine with acyl-alkyl residue sum C36:5, phosphatidylcholine with acyl-alkyl residue sum C38:0, phosphatidylcholine with acyl-alkyl residue sum C38:1, phosphatidylcholine with acyl-alkyl residue sum C38:2, phosphatidylcholine with acyl-alkyl residue sum C38:3, phosphatidylcholine with acyl-alkyl residue sum C38:4, phosphatidylcholine with acyl-alkyl residue sum C38:5, phosphatidylcholine with acyl-alkyl residue sum C38:6, phosphatidylcholine with acyl-alkyl residue sum C40:1, phosphatidylcholine with acyl-alkyl residue sum C40:2, phosphatidylcholine with acyl-alkyl residue sum C40:3, phosphatidylcholine with acyl-alkyl residue sum C40:4, phosphatidylcholine with acyl-alkyl residue sum C40:5, phosphatidylcholine with acyl-alkyl residue sum C40:6, phosphatidylcholine with acyl-alkyl residue sum C42:2, phosphatidylcholine with acyl-alkyl residue sum C42:3, phosphatidylcholine with acyl-alkyl residue sum C42:5, phosphatidylcholine with acyl-alkyl residue sum C44:3, phosphatidylcholine with acyl-alkyl residue sum C44:4, phosphatidylcholine with acyl-alkyl residue sum C44:5, phosphatidylcholine with acyl-alkyl residue sum C44:6, hydroxysphingomyelin with acyl residue sum C14:1, hydroxysphingomyelin with acyl residue sum C16:1, hydroxysphingomyelin with acyl residue sum C22:1, hydroxysphingomyelin with acyl residue sum C22:2, hydroxysphingomyelin with acyl residue sum C24:1, sphingomyelin with acyl residue sum C16:0, sphingomyelin with acyl residue sum C16:1, sphingomyelin with acyl residue sum C18:0, sphingomyelin with acyl residue sum C18:1, sphingomyelin with acyl residue sum C20:2, sphingomyelin with acyl residue sum C24:0, sphingomyelin with acyl residue sum C24:1, sphingomyelin with acyl residue sum C26:0, sphingomyelin with acyl residue sum C26:1, hexoses [including glucose], alanine, arginine, asparagine, aspartate, citrulline, glutamine, glutamate, glycine, histidine, isoleucine, lysine, methionine, ornithine, phenylalanine, proline, serine, threonine, tryptophan, tyrosine, valine, asymmetric dimethylarginine, alpha-aminoadipic acid, creatinine, kynurenine, methionine sulfoxide, putrescine, sarcosine, symmetric dimethylarginine, spermidine, spermine, trans-4-hydroxyproline, or taurine from the biological sample;
and predicting mortality in an individual with sepsis, based at least in part on levels of: adrenoceptor β2 (ADRB2), CD177 molecule (CD177), carboxypeptidase vitellogenic like (CPVL), C-X3-C motif chemokine receptor 1 (CX3CR1), defensin α3 (DEFA3), Fc receptor like 5 (FCRL5), G protein subunit γ2 (GNG2), interleukin 10 receptor subunit α (IL10RA), kinesin light chain 3 (KLC3), oleoyl-ACP hydrolase (OLAH), pyruvate kinase M1/2 (PKM), radical S-adenosyl methionine domain containing 2 (RSAD2), STE20 related adaptor β (STRADB), tyrosylprotein sulfotransferase 1 (TPST1), tetraspanin 5 (TSPAN5), tetratricopeptide repeat domain 9C (TTC9C), zinc finger with KRAB and SCAN domains 1 (ZKSCAN1), C-reactive protein (CRP), C-X-C motif chemokine ligand 10/interferon gamma-induced protein 10 (CXCL10/IP-10), D-dimer, ferritins, fibrinogens, (soluble) intercellular adhesion molecule 1 (ICAM-1), interferon gamma (IFNγ), interleukin-1 receptor antagonist (IL-1RA), interleukin-5 (IL-5), interleukin-6 (IL-6), interleukin-6 receptor α (IL-6Rα), interleukin-18 (IL-18), interleukin-18-binding protein (IL-18BP), lipocalin-2 (LCN-2), matrix metalloproteinase-8 (MMP-8), procalcitonin (PCT), (soluble) receptor for advanced glycation end products (RAGE), tumor necrosis factor receptor 1 (TNF-R1), tumor necrosis factor alpha (TNFα), vascular endothelial growth factors (VEGF), von Willebrand factor A2 domain (vWF-A2), carnitine, acetylcarnitine, propionylcarnitine, malonylcarnitine, methylmalonylcarnitine, hydroxypropionylcarnitine, propenoylcarnitine, butyrylcarnitine, hydroxybutyrylcarnitine, fumarylcarnitine, valerylcarnitine, glutarylcarnitine, hydroxyvalerylcarnitine, tiglylcarnitine, hexanoylcarnitine, hydroxyhexanoylcarnitine, pimeloylcarnitine, decanoylcarnitine, decadienylcarnitine, tetradecenoylcarnitine, hydroxytetradecenoylcarnitine, hydroxytetradecadienylcarnitine, hexadecanoylcarnitine, hexadecenoylcarnitine, octadecanoylcarnitine, octadecenoylcarnitine, lysophosphatidylcholine with acyl residue sum C16:0, lysophosphatidylcholine with acyl residue sum C16:1, lysophosphatidylcholine with acyl residue sum C17:0, lysophosphatidylcholine with acyl residue sum C18:0, lysophosphatidylcholine with acyl residue sum C18:1, lysophosphatidylcholine with acyl residue sum C18:2, lysophosphatidylcholine with acyl residue sum C20:3, lysophosphatidylcholine with acyl residue sum C20:4, lysophosphatidylcholine with acyl residue sum C24:0, lysophosphatidylcholine with acyl residue sum C26:0, lysophosphatidylcholine with acyl residue sum C26:1, lysophosphatidylcholine with acyl residue sum C28:0, lysophosphatidylcholine with acyl residue sum C28:1, phosphatidylcholine with diacyl residue sum C24:0, phosphatidylcholine with diacyl residue sum C28:1, phosphatidylcholine with diacyl residue sum C30:0, phosphatidylcholine with diacyl residue sum C32:0, phosphatidylcholine with diacyl residue sum C32:1, phosphatidylcholine with diacyl residue sum C32:3, phosphatidylcholine with diacyl residue sum C34:1, phosphatidylcholine with diacyl residue sum C34:2, phosphatidylcholine with diacyl residue sum C34:3, phosphatidylcholine with diacyl residue sum C34:4, phosphatidylcholine with diacyl residue sum C36:0, phosphatidylcholine with diacyl residue sum C36:1, phosphatidylcholine with diacyl residue sum C36:2, phosphatidylcholine with diacyl residue sum C36:3, phosphatidylcholine with diacyl residue sum C36:4, phosphatidylcholine with diacyl residue sum C36:5, phosphatidylcholine with diacyl residue sum C36:6, phosphatidylcholine with diacyl residue sum C38:0, phosphatidylcholine with diacyl residue sum C38:3, phosphatidylcholine with diacyl residue sum C38:4, phosphatidylcholine with diacyl residue sum C38:5, phosphatidylcholine with diacyl residue sum C38:6, phosphatidylcholine with diacyl residue sum C40:2, phosphatidylcholine with diacyl residue sum C40:3, phosphatidylcholine with diacyl residue sum C40:4, phosphatidylcholine with diacyl residue sum C40:5, phosphatidylcholine with diacyl residue sum C40:6, phosphatidylcholine with diacyl residue sum C42:0, phosphatidylcholine with diacyl residue sum C42:1, phosphatidylcholine with diacyl residue sum C42:2, phosphatidylcholine with diacyl residue sum C42:4, phosphatidylcholine with diacyl residue sum C42:5, phosphatidylcholine with diacyl residue sum C42:6, phosphatidylcholine with acyl-alkyl residue sum C30:0, phosphatidylcholine with acyl-alkyl residue sum C30:1, phosphatidylcholine with acyl-alkyl residue sum C30:2, phosphatidylcholine with acyl-alkyl residue sum C32:1, phosphatidylcholine with acyl-alkyl residue sum C32:2, phosphatidylcholine with acyl-alkyl residue sum C34:0, phosphatidylcholine with acyl-alkyl residue sum C34:1, phosphatidylcholine with acyl-alkyl residue sum C34:2, phosphatidylcholine with acyl-alkyl residue sum C34:3, phosphatidylcholine with acyl-alkyl residue sum C36:0, phosphatidylcholine with acyl-alkyl residue sum C36:1, phosphatidylcholine with acyl-alkyl residue sum C36:2, phosphatidylcholine with acyl-alkyl residue sum C36:3, phosphatidylcholine with acyl-alkyl residue sum C36:4, phosphatidylcholine with acyl-alkyl residue sum C36:5, phosphatidylcholine with acyl-alkyl residue sum C38:0, phosphatidylcholine with acyl-alkyl residue sum C38:1, phosphatidylcholine with acyl-alkyl residue sum C38:2, phosphatidylcholine with acyl-alkyl residue sum C38:3, phosphatidylcholine with acyl-alkyl residue sum C38:4, phosphatidylcholine with acyl-alkyl residue sum C38:5, phosphatidylcholine with acyl-alkyl residue sum C38:6, phosphatidylcholine with acyl-alkyl residue sum C40:1, phosphatidylcholine with acyl-alkyl residue sum C40:2, phosphatidylcholine with acyl-alkyl residue sum C40:3, phosphatidylcholine with acyl-alkyl residue sum C40:4, phosphatidylcholine with acyl-alkyl residue sum C40:5, phosphatidylcholine with acyl-alkyl residue sum C40:6, phosphatidylcholine with acyl-alkyl residue sum C42:2, phosphatidylcholine with acyl-alkyl residue sum C42:3, phosphatidylcholine with acyl-alkyl residue sum C42:5, phosphatidylcholine with acyl-alkyl residue sum C44:3, phosphatidylcholine with acyl-alkyl residue sum C44:4, phosphatidylcholine with acyl-alkyl residue sum C44:5, phosphatidylcholine with acyl-alkyl residue sum C44:6, hydroxysphingomyelin with acyl residue sum C14:1, hydroxysphingomyelin with acyl residue sum C16:1, hydroxysphingomyelin with acyl residue sum C22:1, hydroxysphingomyelin with acyl residue sum C22:2, hydroxysphingomyelin with acyl residue sum C24:1, sphingomyelin with acyl residue sum C16:0, sphingomyelin with acyl residue sum C16:1, sphingomyelin with acyl residue sum C18:0, sphingomyelin with acyl residue sum C18:1, sphingomyelin with acyl residue sum C20:2, sphingomyelin with acyl residue sum C24:0, sphingomyelin with acyl residue sum C24:1, sphingomyelin with acyl residue sum C26:0, sphingomyelin with acyl residue sum C26:1, hexoses [including glucose], alanine, arginine, asparagine, aspartate, citrulline, glutamine, glutamate, glycine, histidine, isoleucine, lysine, methionine, ornithine, phenylalanine, proline, serine, threonine, tryptophan, tyrosine, valine, asymmetric dimethylarginine, alpha-aminoadipic acid, creatinine, kynurenine, methionine sulfoxide, putrescine, sarcosine, symmetric dimethylarginine, spermidine, spermine, trans-4-hydroxyproline, or taurine.

31. The method of any one of claims 1-19 or 30, wherein the method further comprises treating the individual for sepsis or preventing the development of sepsis.
